(19) United States
(12) Patent Application Publication
(10) Pub. No.: US 2024/0254076 A1
(43) Pub. Date: Aug. 1, 2024

(54) LIPID NANOMATERIALS AND USES THEREOF

(71) Applicant: Ohio State Innovation Foundation, Columbus, OH (US)

(72) Inventors: Yizhou DONG, Dublin, OH (US); Chang WANG, Dublin, OH (US); Yuebao ZHANG, Dublin, OH (US); Yonger XUE, Columbus, OH (US)

(21) Appl. No.: 18/557,218

(22) PCT Filed: Apr. 26, 2022
(86) PCT No.: PCT/US2022/026413

§ 371 Date: Oct. 25, 2023

Related U.S. Application Data
(60) Provisional application No. 63/179,688, filed on Apr. 26, 2021.

(51) Int. Cl. C07C 237/20; A61K 9/16; A61K 9/48; A61K 47/18; A61K 47/20; A61K 48/00; C07C 211/38; C07C 211/49; C07C 237/08; C07C 317/30; C07D 209/20; C07D 401/04; C07D 487/04

(52) U.S. Cl. CPC C07C 237/20; A61K 9/1617; A61K 9/4858; A61K 47/18; A61K 47/20; A61K 48/0008; C07C 211/38; C07C 211/49; C07C 237/08; C07C 317/30; C07D 209/20; C07D 401/04; C07D 487/04

(57) ABSTRACT

Disclosed herein are lipid formulations that can be used in drug delivery and screening.

### CROSS REFERENCE TO RELATED APPLICATIONS

This application claims the benefit of U.S. Provisional patent application Ser. No. 63/179,688 filed Apr. 26, 2021, the disclosure of which is expressly incorporated herein by reference.

### STATEMENT REGARDING GOVERNMENT SUPPORT

This invention was made with government support under grant/contract number R35 GM119679 awarded by the National Institutes of Health. The government has certain rights in the invention.

### TECHNICAL FIELD

This application generally relates to lipid formulations that can be used in drug delivery and screening.

### BACKGROUND

Efficient delivery of therapeutic agents, such as mRNA, is a key step and challenge in mRNA therapeutics. These nucleic acid-based therapeutics have demonstrated enormous potential as a means to preserve function of the therapeutic agent, but there remains a need for more effective delivery to appropriate sites within a cell or organism in order to realize this potential. Despite promising data from ongoing clinical trials, the clinical use of mRNA requires the discovery and development of more efficient delivery systems. Because the efficacy of these delivery systems typical stems from the compositional structure of the base lipid molecule, new compositions and methods are needed for delivering mRNA to cells for treating various disease states.

However, there are currently problems restricting the widespread use of oligonucleotides in therapeutic and diagnostic contexts. First, free RNAs are susceptible to nuclease digestion in plasma, facilitating degradation of the therapeutic agent. Second, these oligonucleotides are often limited in accessing the intracellular compartment where the relevant translation machinery resides. Lipid nanoparticles formed from cationic lipids with other lipid components have been used to traverse these barriers and increase the cellular uptake of these oligonucleotides. Thus, there remains a need for improved cationic lipids and lipid nanoparticles for the delivery of oligonucleotides.

### SUMMARY

In accordance with the purposes of the disclosed compounds and methods as embodied and broadly described herein, the disclosed subject matter relates to compounds and methods of making and using thereof.

For example, disclosed herein are compounds defined by Formulas I-XIX, or pharmaceutically acceptable salts thereof:

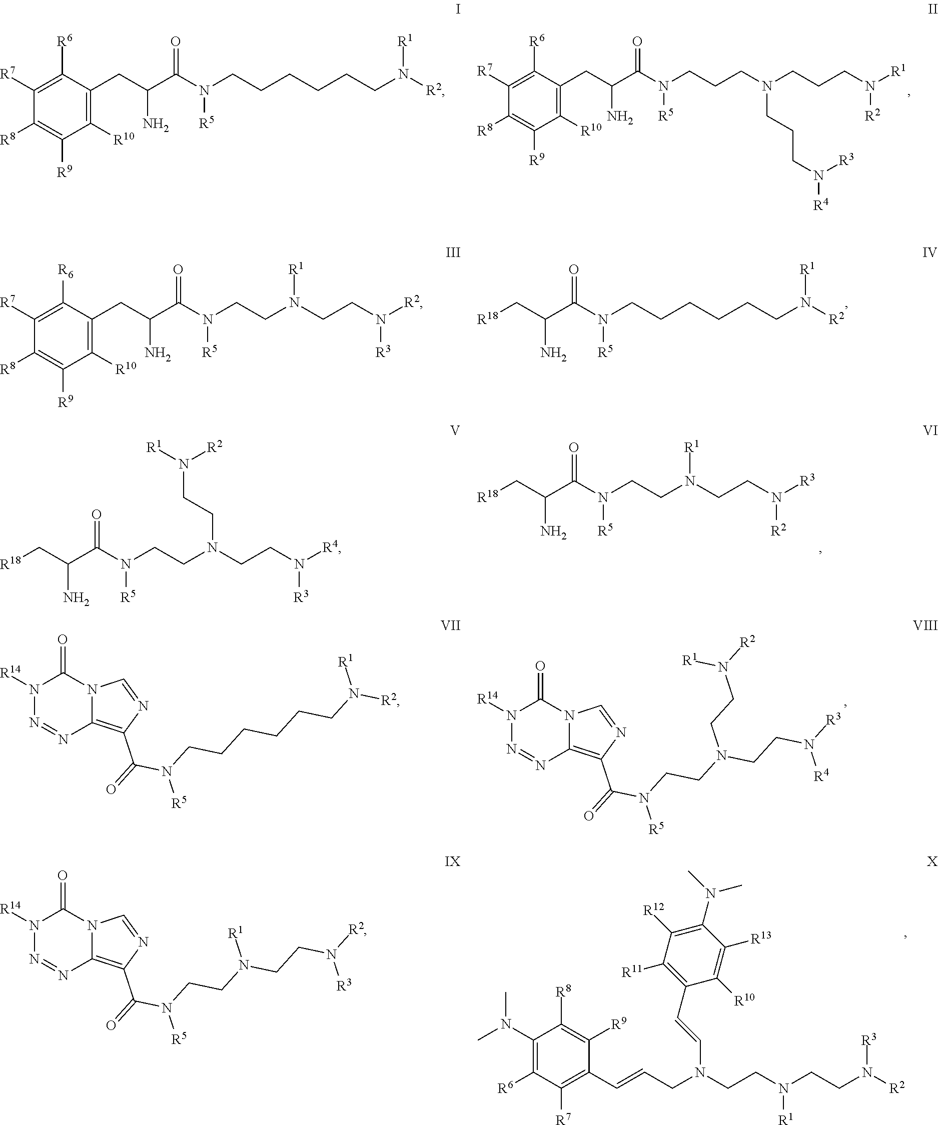

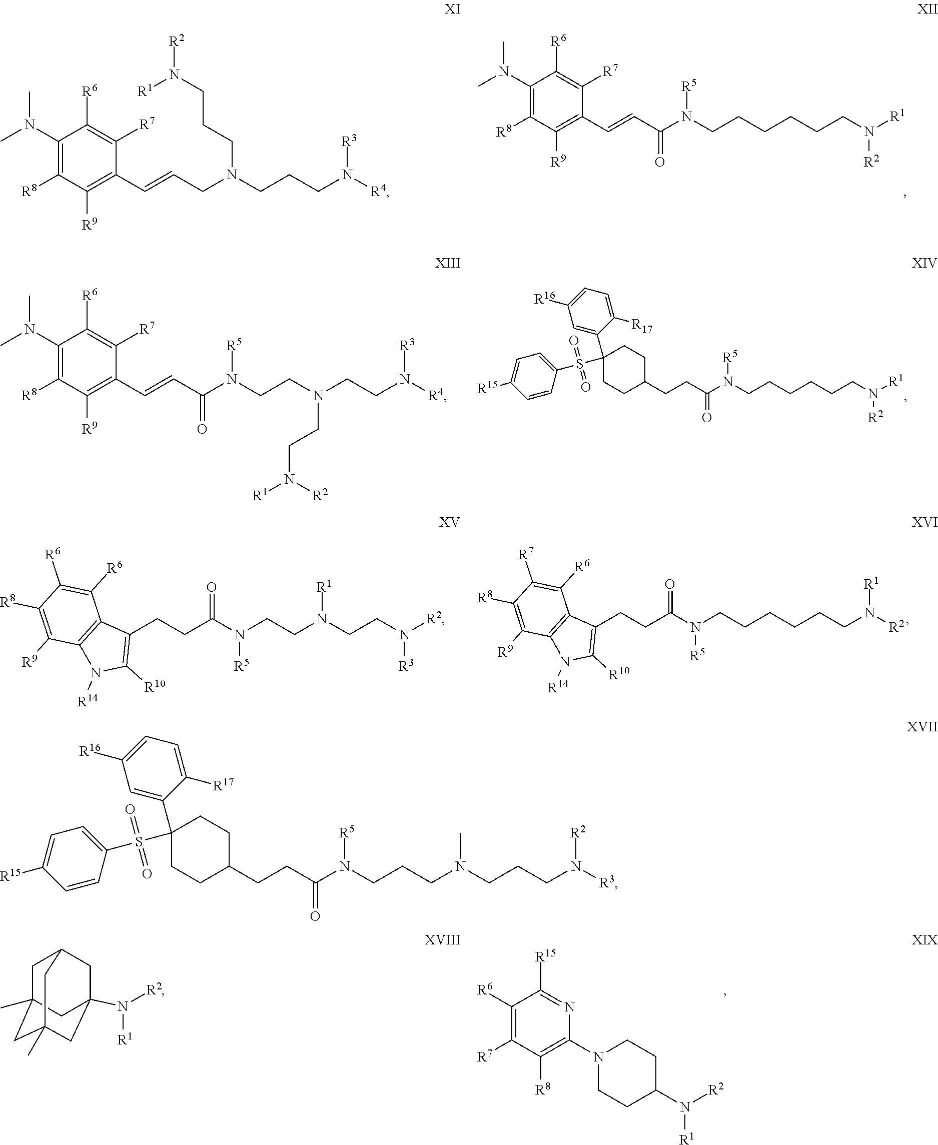

wherein
R¹, R², R³, and R⁴ are each independently a substituted or unsubstituted C₁-C₂₀ alkyl;
R⁵ and R¹⁴ are each independently hydrogen, a substituted or unsubstituted C₁-C₅ alkyl;
R⁶, R⁷, R⁸, R⁹, R¹⁰, R¹¹, R¹², and R¹³ are each independently hydrogen, OH, halogen, or substituted or unsubstituted C₁-C₅ alkyl;
R¹⁵, R¹⁶, and R¹⁷ are each independently hydrogen, OH, halogen, or substituted or unsubstituted C₁-C₅ alkyl; and
R¹⁸ is OH or substituted or unsubstituted C₁-C₁₀ alkyl.

In some aspects, R¹, R², R³, and R⁴ are each independently selected from the group consisting of:

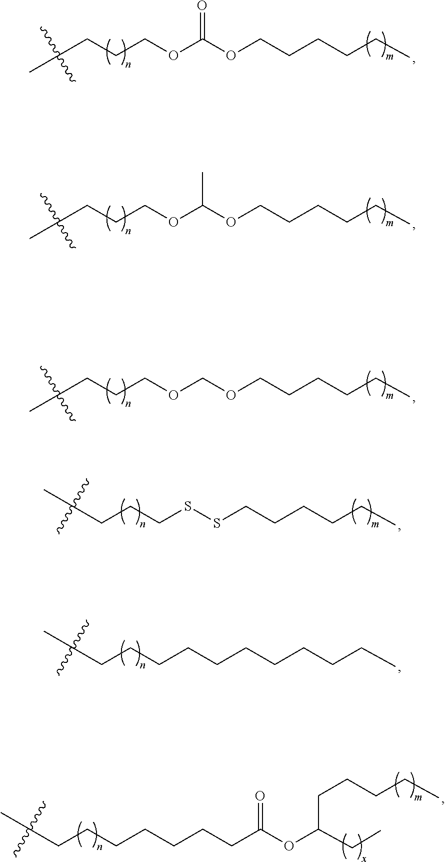

wherein n, m, and x each independently represent integers from 1 to 9, such as from 1 to 5.

Also disclosed herein are methods of making any of the compositions disclosed herein.

Also disclosed herein are lipid particles comprising any of the compositions disclosed herein. In some examples, the lipid particle is substantially spherical in shape. In some examples, the lipid particle has an average particle size of from 50 nanometers (nm) to 500 nm. In some examples, the lipid particle has an average particle size of from 100 nm to 200 nm, from 120 nm to 140 nm, or from 150 nm to 200 nm. In some examples, the lipid particle has a polydispersity index of 0.3 or less, 0.2 or less, or 0.1 or less. In some examples, the lipid particle further comprises an additional component. In some examples, the additional component comprises an additional lipid. In some examples, the additional lipid comprises a phospholipid, a sterol, or a combination thereof. In some examples, the lipid particle further comprises 1,2-dioleoyl-sn-glycero-3-phosphoethanolamine (DOPE), cholesterol, 1,2-dimyristoyl-rac-glycero-3-methylpolyoxyethylene, or a combination thereof.

Also disclosed herein are pharmaceutical compositions comprising a therapeutic agent encapsulated within any of the lipid particles disclosed herein. In some examples, the therapeutic agent is encapsulated within the lipid particle with an encapsulation efficiency of 50% or more, 75% or more, or 90% or more. In some examples, the therapeutic agent comprises an anticancer agent, an anti-inflammatory agent, an antimicrobial agent, or a combination thereof. In some examples, the therapeutic agent comprises a chemotherapeutic agent, an immunotherapeutic agent, or a combination thereof. In some examples, the therapeutic agent comprises a nucleic acid. In some examples, the nucleic acid is mRNA.

Also disclosed herein are methods of treating a disease or disorder in a subject in need thereof, the methods comprising administering to the subject a therapeutically effective amount of any of the pharmaceutical compositions disclosed herein. In some examples, the disease comprises a neurological disease. In other aspects, the disease comprises a hepatic disease. In further aspects, the disease comprises a musculoskeletal disease.

Additional advantages of the disclosed compositions and methods will be set forth in part in the description which follows, and in part will be obvious from the description. The advantages of the disclosed compositions and methods will be realized and attained by means of the elements and combinations particularly pointed out in the appended claims. It is to be understood that both the foregoing general description and the following detailed description are exemplary and explanatory only and are not restrictive of the disclosed compositions and methods, as claimed.

The details of one or more embodiments of the invention are set forth in the accompanying drawings and the description below. Other features, objects, and advantages of the invention will be apparent from the description and drawings, and from the claims.

### BRIEF DESCRIPTION OF THE FIGURES

The accompanying figures, which are incorporated in and constitute a part of this specification, illustrate several aspects of the disclosure, and together with the description, serve to explain the principles of the disclosure.

FIG. 1 depicts formulation and screening of brain-targeting lipid nanoparticles (LNPs) encapsulating firefly luciferase mRNA in CCL131 cells, a mouse neuroblast cell line using. Data was normalized with MC3 LNP.

FIG. 2 depicts a cluster-testing method to examine mRNA delivery in vivo using brain targeting lipids (BL28, BL39, BL54, BL68 and BL70) formulated LNP. Compared with the PBS-treated group, the clustered LNP-mRNA formulation showed significantly higher bioluminescence intensity in mouse brain.

FIG. 3 depicts a collection of samples compounds forming the basis of lipid nanoparticles.

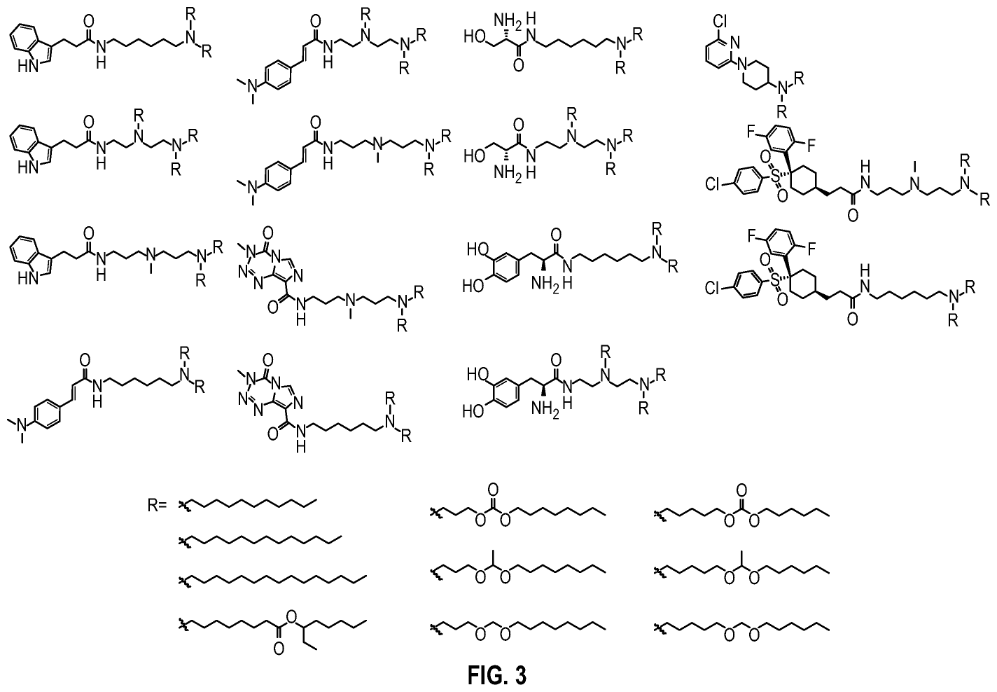

### DETAILED DESCRIPTION

Reference will now be made in detail to the embodiments of the disclosure. The present disclosure may, however, be embodied in many different forms and should not be construed as limited to the embodiments set forth herein.

Unless defined otherwise, all technical and scientific terms used herein have the same meaning as commonly understood to one of ordinary skill in the art to which this invention belongs. The following definitions are provided for the full understanding of terms used in this specification.

### General Definitions

As used in the specification and claims, the singular form “a,” “an,” and “the” include plural references unless the context clearly dictates otherwise. For example, the term “an agent” includes a plurality of agents, including mixtures thereof.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

As used herein, “administration” of an agent to a subject includes any route of introducing or delivering to a subject an agent to perform the agent's intended function(s). Administration can be carried out by any suitable route, including orally, intranasally, parenterally (intravenously, intramuscularly, intraperitoneally, or subcutaneously), topically, and the like. “Systemic administration” refers to the introducing or delivering to a subject an agent via a route which introduces or delivers the agent to extensive areas of the subject's body (e.g. greater than 50% of the body), for example through entrance into the circulatory, gastrointestinal, or lymphatic systems. By contrast, “local administration” refers to the introducing or delivery to a subject an agent via a route which introduces or delivers the agent to the area or areas within the local vicinity of the point of administration and does not introduce the agent systemically in a therapeutically significant amount. For example, locally administered agents are easily detectable in the local vicinity of the point of administration, but are undetectable or detectable at negligible amounts in distal parts of the subject's body. Administration includes self-administration and the administration by another.

As used herein, the term “preventing” a disorder or unwanted physiological event in a subject refers specifically to the prevention of the progression or recurrence of symptoms and/or their underlying cause, wherein the subject may or may not exhibit heightened susceptibility to the disorder or event (for example, either inducing regression or preventing progression).

The term “effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts.

An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation. For example, an effective amount of an agent may vary from about 0.001 mg/kg to about 1000 mg/kg in one or more dose administrations for one or several days (depending on the mode of administration). In certain embodiments, the effective amount per dose varies from about 0.001 mg/kg to about 1000 mg/kg, from about 0.01 mg/kg to about 750 mg/kg, from about 0.1 mg/kg to about 500 mg/kg, from about 1.0 mg/kg to about 250 mg/kg, and from about 10.0 mg/kg to about 150 mg/kg.

As used herein, the term “pharmaceutically acceptable” component can refer to a component that is not biologically or otherwise undesirable, e.g., the component may be incorporated into a pharmaceutical formulation of the invention and administered to a subject as described herein without causing any significant undesirable biological effects or interacting in a deleterious manner with any of the other components of the formulation in which it is contained. When the term “pharmaceutically acceptable” is used to refer to an excipient, it is generally implied that the component has met the required standards of toxicological and manufacturing testing or that it is included on the Inactive Ingredient Guide prepared by the U.S. Food and Drug Administration.

As used herein, the term “pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

As used herein, the term “controlled release” refers to release of an agent from a given dosage form in a controlled fashion in order to achieve the desired pharmacokinetic profile in vivo. An aspect of “controlled release” agent delivery is the ability to manipulate the formulation and/or dosage form in order to establish the desired kinetics of agent release.

As used herein, the term “subject” or “host” can refer to living organisms such as mammals, including, but not limited to humans, livestock, dogs, cats, and other mammals. Administration of the therapeutic agents can be carried out at dosages and for periods of time effective for treatment of a subject. In some embodiments, the subject is a human.

Ranges can be expressed herein as from “about” one particular value, and/or to “about” another particular value. By “about” is meant within 5% of the value, e.g., within 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

“Exemplary” means “an example of” and is not intended to convey an indication of a preferred or ideal embodiment. “Such as” is not used in a restrictive sense, but for explanatory purposes.

Values can be expressed herein as an “average” value. “Average” generally refers to the statistical mean value.

By “substantially” is meant within 5%, e.g., within 4%, 3%, 2%, or 1%.

It is understood that throughout this specification the identifiers “first” and “second” are used solely to aid in distinguishing the various components and steps of the disclosed subject matter. The identifiers “first” and “second” are not intended to imply any particular order, amount, preference, or importance to the components or steps modified by these terms.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

The term “inhibit” refers to a decrease in an activity, response, condition, disease, or other biological parameter. This can include but is not limited to the complete ablation of the activity, response, condition, or disease. This can also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. By way of example, in the context of fibrotic conditions, “treating,” “treat,” and “treatment” as used herein, refers to partially or completely inhibiting or reducing the fibrotic condition which the subject is suffering. In one embodiment, this term refers to an action that occurs while a patient is suffering from, or is diagnosed with, the fibrotic condition, which reduces the severity of the condition, or retards or slows the progression of the condition. Treatment need not result in a complete cure of the condition; partial inhibition or reduction of the fibrotic condition is encompassed by this term.

The term “anticancer” refers to the ability to treat or control cellular proliferation and/or tumor growth at any concentration.

As used herein, “molecular weight” refers to number average molecular weight as measured by ¹H NMR spectroscopy, unless indicated otherwise.

As used herein, the term “delivery” encompasses both local and systemic delivery. For example, delivery of mRNA encompasses situations in which an mRNA is delivered to a target tissue and the encoded protein or peptide is expressed and retained within the target tissue (also referred to as “local distribution” or “local delivery”), and situations in which an mRNA is delivered to a target tissue and the encoded protein or peptide is expressed and secreted into patient's circulation system (e.g., serum) and systematically distributed and taken up by other tissues (also referred to as “systemic distribution” or “systemic delivery).

As used herein, the term “encapsulation,” or grammatical equivalent, refers to the process of confining an individual nucleic acid molecule within a nanoparticle.

As used herein, “expression” of a mRNA refers to translation of an mRNA into a peptide (e.g., an antigen), polypeptide, or protein (e.g., an enzyme) and also can include, as indicated by context, the post-translational modification of the peptide, polypeptide or fully assembled protein (e.g., enzyme). In this application, the terms “expression” and “production,” and grammatical equivalent, are used inter-changeably.

As used herein, the term “messenger RNA (mRNA)” refers to a polynucleotide that encodes at least one peptide, polypeptide or protein. mRNA as used herein encompasses both modified and unmodified RNA. mRNA may contain one or more coding and non-coding regions. mRNA can be purified from natural sources, produced using recombinant expression systems and optionally purified, chemically synthesized, etc. Where appropriate, e.g., in the case of chemically synthesized molecules, mRNA can comprise nucleoside analogs such as analogs having chemically modified bases or sugars, backbone modifications, etc. An mRNA sequence is presented in the 5′ to 3′ direction unless otherwise indicated. In some embodiments, an mRNA is or comprises natural nucleosides (e.g., adenosine, guanosine, cytidine, uridine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, pseudouridine, and 5-methylcytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

As used herein, the term “nucleic acid,” in its broadest sense, refers to any compound and/or substance that is or can be incorporated into a polynucleotide chain. In some embodiments, a nucleic acid is a compound and/or substance that is or can be incorporated into a polynucleotide chain via a phosphodiester linkage. In some embodiments, “nucleic acid” refers to individual nucleic acid residues (e.g., nucleotides and/or nucleosides). In some embodiments, “nucleic acid” refers to a polynucleotide chain comprising individual nucleic acid residues. In some embodiments, “nucleic acid” encompasses RNA as well as single and/or double-stranded DNA and/or cDNA. Furthermore, the terms “nucleic acid,” “DNA,” “RNA,” and/or similar terms include nucleic acid analogs, i.e., analogs having other than a phosphodiester backbone.

### Chemical Definitions

Unless otherwise defined, all technical and scientific terms used herein have the same meaning as commonly understood by one of ordinary skill in the art to which this invention belongs.

The organic moieties mentioned when defining variable positions within the general formulae described herein (e.g., the term “halogen”) are collective terms for the individual substituents encompassed by the organic moiety. The prefix Cₙ-Cₘ preceding a group or moiety indicates, in each case, the possible number of carbon atoms in the group or moiety that follows.

The term “ion,” as used herein, refers to any molecule, portion of a molecule, cluster of molecules, molecular complex, moiety, or atom that contains a charge (positive, negative, or both at the same time within one molecule, cluster of molecules, molecular complex, or moiety (e.g., zwitterions)) or that can be made to contain a charge. Methods for producing a charge in a molecule, portion of a molecule, cluster of molecules, molecular complex, moiety, or atom are disclosed herein and can be accomplished by methods known in the art, e.g., protonation, deprotonation, oxidation, reduction, alkylation, acetylation, esterification, de-esterification, hydrolysis, etc.

The term “anion” is a type of ion and is included within the meaning of the term “ion.” An “anion” is any molecule, portion of a molecule (e.g., zwitterion), cluster of molecules, molecular complex, moiety, or atom that contains a net negative charge or that can be made to contain a net negative charge. The term “anion precursor” is used herein to specifically refer to a molecule that can be converted to an anion via a chemical reaction (e.g., deprotonation).

The term “cation” is a type of ion and is included within the meaning of the term “ion.” A “cation” is any molecule, portion of a molecule (e.g., zwitterion), cluster of molecules, molecular complex, moiety, or atom, that contains a net positive charge or that can be made to contain a net positive charge. The term “cation precursor” is used herein to specifically refer to a molecule that can be converted to a cation via a chemical reaction (e.g., protonation or alkylation).

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valencies of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

“Z¹,” “Z²,” “Z³,” and “Z⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” as used herein refers to a non-aromatic hydrocarbon group and includes branched and unbranched, alkyl, alkenyl, or alkynyl groups.

As used herein, the term “alkyl” refers to saturated, straight-chained or branched saturated hydrocarbon moieties. Unless otherwise specified, C₁-C₂₄ (e.g., C₂-C₂₂, C₄-C₂₂, C₆-C₂₂, C₈-C₂₂, C₁₀-C₂₂, C₁₂-C₂₂, C₁₄-C₂₂, C₁₆-C₂₂, C₂-C₂₀, C₄-C₂₀, C₆-C₂₀, C₈-C₂₀, C₁₀- C₂₀, C₁₂-C₂₀, C₁₄-C₂₀, C₁₆-C₂₀, C₁-C₂₀, C₁-C₁₈, C₁-C₁₆, C₁-C₁₄, C₁-C₁₂, C₁-C₁₀, C₁-C₈, C₁-C₆, or C₁-C₄) alkyl groups are intended. Examples of alkyl groups include methyl, ethyl, propyl, 1-methyl-ethyl, butyl, 1-methyl-propyl, 2-methyl-propyl, 1,1-dimethyl-ethyl, pentyl, 1-methyl-butyl, 2-methyl-butyl, 3-methyl-butyl, 2,2-dimethyl-propyl, 1-ethyl-propyl, hexyl, 1,1-dimethyl-propyl, 1,2-dimethyl-propyl, 1-methyl-pentyl, 2-methyl-pentyl, 3-methyl-pentyl, 4-methyl-pentyl, 1,1-dimethyl-butyl, 1,2-dimethyl-butyl, 1,3-dimethyl-butyl, 2,2-dimethyl-butyl, 2,3-dimethyl-butyl, 3,3-dimethyl-butyl, 1-ethyl-butyl, 2-ethyl-butyl, 1,1,2-trimethyl-propyl, 1,2,2-trimethyl-propyl, 1-ethyl-1-methyl-propyl, 1-ethyl-2-methyl-propyl, heptyl, octyl, nonyl, decyl, dodecyl, tetradecyl, hexadecyl, eicosyl, tetracosyl, and the like. Alkyl substituents may be unsubstituted or substituted with one or more chemical moieties. The alkyl group can be substituted with one or more groups including, but not limited to, hydroxyl, halogen, acetal, acyl, alkyl, alkoxy, alkenyl, alkynyl, aryl, heteroaryl, aldehyde, amino, cyano, carboxylic acid, ester, ether, carbonate ester, carbamate ester, ketone, nitro, phosphonyl, silyl, sulfo-oxo, sulfonyl, sulfone, sulfoxide, or thiol, as described below, provided that the substituents are sterically compatible and the rules of chemical bonding and strain energy are satisfied.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halides (halogens; e.g., fluorine, chlorine, bromine, or iodine). The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

As used herein, the term “alkenyl” refers to unsaturated, straight-chained, or branched hydrocarbon moieties containing a double bond. Unless otherwise specified, C₂-C₂₄ (e.g., C₂-C₂₂, C₂-C₂₀, C₂-C₁₈, C₂-C₁₆, C₂-C₁₄, C₂-C₁₂, C₂-C₁₀, C₂-C₈, C₂-C₆, or C₂-C₄) alkenyl groups are intended. Alkenyl groups may contain more than one unsaturated bond. Examples include ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, and 1-ethyl-2-methyl-2-propenyl. The term “vinyl” refers to a group having the structure —CH═CH₂; 1-propenyl refers to a group with the structure —CH═CH—CH₃; and 2-propenyl refers to a group with the structure —CH₂—CH═CH₂. Asymmetric structures such as (Z¹Z²)C═C(Z³Z⁴) are intended to include both the E and Z isomers. This can be presumed in structural formulae herein wherein an asymmetric alkene is present, or it can be explicitly indicated by the bond symbol C═C. Alkenyl substituents may be unsubstituted or substituted with one or more chemical moieties. Examples of suitable substituents include, for example, alkyl, alkoxy, alkenyl, alkynyl, aryl, heteroaryl, acetal, acyl, aldehyde, amino, cyano, carboxylic acid, ester, ether, carbonate ester, carbamate ester, halide, hydroxyl, ketone, nitro, phosphonyl, silyl, sulfo-oxo, sulfonyl, sulfone, sulfoxide, or thiol, as described below, provided that the substituents are sterically compatible and the rules of chemical bonding and strain energy are satisfied.

As used herein, the term “alkynyl” represents straight-chained or branched hydrocarbon moieties containing a triple bond. Unless otherwise specified, C₂-C₂₄ (e.g., C₂-C₂₄, C₂-C₂₀, C₂-C₁₈, C₂-C₁₆, C₂-C₁₄, C₂-C₁₂, C₂-C₁₀, C₂-C₈, C₂-C₆, or C₂-C₄) alkynyl groups are intended. Alkynyl groups may contain more than one unsaturated bond. Examples include C₂-C₆-alkynyl, such as ethynyl, 1-propynyl, 2-propynyl (or propargyl), 1-butynyl, 2-butynyl, 3-butynyl, 1-methyl-2-propynyl, 1-pentynyl, 2-pentynyl, 3-pentynyl, 4-pentynyl, 3-methyl-1-butynyl, 1-methyl-2-butynyl, 1-methyl-3-butynyl, 2-methyl-3-butynyl, 1,1-dimethyl-2-propynyl, 1-ethyl-2-propynyl, 1-hexynyl, 2-hexynyl, 3-hexynyl, 4-hexynyl, 5-hexynyl, 3-methyl-1-pentynyl, 4-methyl-1-pentynyl, 1-methyl-2-pentynyl, 4-methyl-2-pentynyl, 1-methyl-3-pentynyl, 2-methyl-3-pentynyl, 1-methyl-4-pentynyl, 2-methyl-4-pentynyl, 3-methyl-4-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, I-ethyl-2-butynyl, 1-ethyl-3-butynyl, 2-ethyl-3-butynyl, and 1-ethyl-1-methyl-2-propynyl. Alkynyl substituents may be unsubstituted or substituted with one or more chemical moieties. Examples of suitable substituents include, for example, alkyl, alkoxy, alkenyl, alkynyl, aryl, heteroaryl, acetal, acyl, aldehyde, amino, cyano, carboxylic acid, ester, ether, carbonate ester, carbamate ester, halide, hydroxyl, ketone, nitro, phosphonyl, silyl, sulfo-oxo, sulfonyl, sulfone, sulfoxide, or thiol, as described below.

As used herein, the term “aryl,” as well as derivative terms such as aryloxy, refers to groups that include a monovalent aromatic carbocyclic group of from 3 to 50 carbon atoms. Aryl groups can include a single ring or multiple condensed rings. In some embodiments, aryl groups include C₆-C₁₀ aryl groups. Examples of aryl groups include, but are not limited to, benzene, phenyl, biphenyl, naphthyl, tetrahydronaphthyl, phenylcyclopropyl, phenoxybenzene, and indanyl. The term “aryl” also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus. The term “non-heteroaryl,” which is also included in the term “aryl,” defines a group that contains an aromatic group that does not contain a heteroatom. The aryl substituents may be unsubstituted or substituted with one or more chemical moieties. Examples of suitable substituents include, for example, alkyl, alkoxy, alkenyl, alkynyl, aryl, heteroaryl, acetal, acyl, aldehyde, amino, cyano, carboxylic acid, ester, ether, carbonate ester, carbamate ester, halide, hydroxyl, ketone, nitro, phosphonyl, silyl, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms, e.g., 3 to 10. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, acetal, acyl, aldehyde, amino, cyano, carboxylic acid, ester, ether, carbonate ester, carbamate ester, halide, hydroxyl, ketone, nitro, phosphonyl, silyl, sulfo-oxo, sulfonyl, sulfone, sulfoxide, or thiol as described herein.

The term “cycloalkenyl” as used herein is a non-aromatic carbon-based ring composed of at least three carbon atoms, e.g., 3 to 10, and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above and is included within the meaning of the term “cycloalkenyl,” where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, acetal, acyl, aldehyde, amino, cyano, carboxylic acid, ester, ether, carbonate ester, carbamate ester, halide, hydroxyl, ketone, nitro, phosphonyl, silyl, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems (e.g., monocyclic, bicyclic, tricyclic, polycyclic, etc.) that can be substituted or unsubstituted. A cyclic group can contain one or more aryl groups, one or more non-aryl groups, or one or more aryl groups and one or more non-aryl groups.

The term “acyl” as used herein is represented by the formula —C(O)Z¹ where Z¹ can be a hydrogen, hydroxyl, alkoxy, alkyl, alkenyl, alkynyl, aryl, heteroaryl, cycloalkyl, cycloalkenyl, heterocycloalkyl, or heterocycloalkenyl group described above. As used herein, the term “acyl” can be used interchangeably with “carbonyl.” Throughout this specification “C(O)” or “CO” is a shorthand notation for C═O.

The term “acetal” as used herein is represented by the formula (Z¹Z²)C(═OZ³)(═OZ⁴), where Z¹, Z², Z³, and Z⁴ can be, independently, a hydrogen, halogen, hydroxyl, alkyl, alkenyl, alkynyl, aryl, heteroaryl, cycloalkyl, cycloalkenyl, heterocycloalkyl, or heterocycloalkenyl group described above.

The term “alkanol” as used herein is represented by the formula Z¹OH, where Z¹ can be an alkyl, alkenyl, alkynyl, aryl, heteroaryl, cycloalkyl, cycloalkenyl, heterocycloalkyl, or heterocycloalkenyl group described above.

As used herein, the term “alkoxy” as used herein is an alkyl group bound through a single, terminal ether linkage; that is, an “alkoxy” group can be defined as to a group of the formula Z¹—O—, where Z¹ is unsubstituted or substituted alkyl as defined above. Unless otherwise specified, alkoxy groups wherein Z¹ is a C₁-C₂₄ (e.g., C₁-C₂₂, C₁-C₂₀, C₁-C₁₈, C₁-C₁₆, C₁-C₁₄, C₁-C₁₂, C₁-C₁₀, C₁-C₈, C₁-C₆, or C₁-C₄) alkyl group are intended. Examples include methoxy, ethoxy, propoxy, 1-methyl-ethoxy, butoxy, 1-methyl-propoxy, 2-methyl-propoxy, 1,1-dimethyl-ethoxy, pentoxy, 1-methyl-butyloxy, 2-methyl-butoxy, 3-methyl-butoxy, 2,2-di-methyl-propoxy, 1-ethyl-propoxy, hexoxy, 1,1-dimethyl-propoxy, 1,2-dimethyl-propoxy, 1-methyl-pentoxy, 2-methyl-pentoxy, 3-methyl-pentoxy, 4-methyl-phenoxy, 1,1-dimethyl-butoxy, 1,2-dimethyl-butoxy, 1,3-dimethyl-butoxy, 2,2-dimethyl-butoxy, 2,3-dimethyl-butoxy, 3,3-dimethyl-butoxy, 1-ethyl-butoxy, 2-ethylbutoxy, 1,1,2-trimethyl-propoxy, 1,2,2-trimethyl-propoxy, 1-ethyl-1-methyl-propoxy, and 1-ethyl-2-methyl-propoxy.

The term “aldehyde” as used herein is represented by the formula —C(O)H. Throughout this specification “C(O)” is a shorthand notation for C═O.

The terms “amine” or “amino” as used herein are represented by the formula —NZ¹Z²Z³, where Z¹, Z², and Z³ can each be substitution group as described herein, such as hydrogen, an alkyl, alkenyl, alkynyl, aryl, heteroaryl, cycloalkyl, cycloalkenyl, heterocycloalkyl, or heterocycloalkenyl group described above.

The terms “amide” or “amido” as used herein are represented by the formula —C(O)NZ¹Z², where Z¹ and Z² can each be substitution group as described herein, such as hydrogen, an alkyl, alkenyl, alkynyl, aryl, heteroaryl, cycloalkyl, cycloalkenyl, heterocycloalkyl, or heterocycloalkenyl group described above.

The term “anhydride” as used herein is represented by the formula Z¹C(O)OC(O)Z² where Z¹ and Z², independently, can be an alkyl, alkenyl, alkynyl, aryl, heteroaryl, cycloalkyl, cycloalkenyl, heterocycloalkyl, or heterocycloalkenyl group described above.

The term “cyclic anhydride” as used herein is represented by the formula:

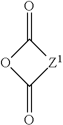

where Z¹ can be an alkyl, alkenyl, alkynyl, aryl, heteroaryl, cycloalkyl, cycloalkenyl, heterocycloalkyl, or heterocycloalkenyl group described above.

The term “azide” as used herein is represented by the formula —N═N═N.

The term “carboxylic acid” as used herein is represented by the formula —C(O)OH.

A “carboxylate” or “carboxyl” group as used herein is represented by the formula —C(O)O⁻.

A “carbonate ester” group as used herein is represented by the formula Z¹OC(O)OZ².

The term “cyano” as used herein is represented by the formula —CN.

The term “ester” as used herein is represented by the formula —OC(O)Z¹ or —C(O)OZ¹, where Z¹ can be an alkyl, alkenyl, alkynyl, aryl, heteroaryl, cycloalkyl, cycloalkenyl, heterocycloalkyl, or heterocycloalkenyl group described above.

The term “ether” as used herein is represented by the formula Z¹OZ², where Z¹ and Z² can be, independently, an alkyl, alkenyl, alkynyl, aryl, heteroaryl, cycloalkyl, cycloalkenyl, heterocycloalkyl, or heterocycloalkenyl group described above.

The term “epoxy” or “epoxide” as used herein refers to a cyclic ether with a three atom ring and can represented by the formula:

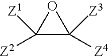

where Z¹, Z², Z³, and Z⁴ can be, independently, an alkyl, alkenyl, alkynyl, aryl, heteroaryl, cycloalkyl, cycloalkenyl, heterocycloalkyl, or heterocycloalkenyl group described above

The term “ketone” as used herein is represented by the formula Z¹C(O)Z², where Z¹ and Z² can be, independently, an alkyl, alkenyl, alkynyl, aryl, heteroaryl, cycloalkyl, cycloalkenyl, heterocycloalkyl, or heterocycloalkenyl group described above.

The term “halide” or “halogen” or “halo” as used herein refers to fluorine, chlorine, bromine, and iodine.

The term “hydroxyl” as used herein is represented by the formula —OH.

The term “nitro” as used herein is represented by the formula —NO₂.

The term “phosphonyl” is used herein to refer to the phospho-oxo group represented by the formula —P(O)(OZ¹)₂, where Z¹ can be hydrogen, an alkyl, alkenyl, alkynyl, aryl, heteroaryl, cycloalkyl, cycloalkenyl, heterocycloalkyl, or heterocycloalkenyl group described above.

The term “silyl” as used herein is represented by the formula —SiZ¹Z²Z³, where Z¹, Z², and Z³ can be, independently, hydrogen, alkyl, alkoxy, alkenyl, alkynyl, aryl, heteroaryl, cycloalkyl, cycloalkenyl, heterocycloalkyl, or heterocycloalkenyl group described above.

The term “sulfonyl” or “sulfone” is used herein to refer to the sulfo-oxo group represented by the formula —S(O)₂Z′, where Z¹ can be hydrogen, an alkyl, alkenyl, alkynyl, aryl, heteroaryl, cycloalkyl, cycloalkenyl, heterocycloalkyl, or heterocycloalkenyl group described above.

The term “sulfide” as used herein is comprises the formula —S—.

The term “thiol” as used herein is represented by the formula —SH.

“R¹,” “R²,” “R³,” “Rⁿ,” etc., where n is some integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an amine group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

Unless stated to the contrary, a formula with chemical bonds shown only as solid lines and not as wedges or dashed lines contemplates each possible stereoisomer or mixture of stereoisomer (e.g., each enantiomer, each diastereomer, each meso compound, a racemic mixture, or scalemic mixture).

### Compounds

Various compounds are disclosed herein. For example, disclosed herein are compounds represented by any one of Formulas I-XIX, or pharmaceutically acceptable salts thereof:

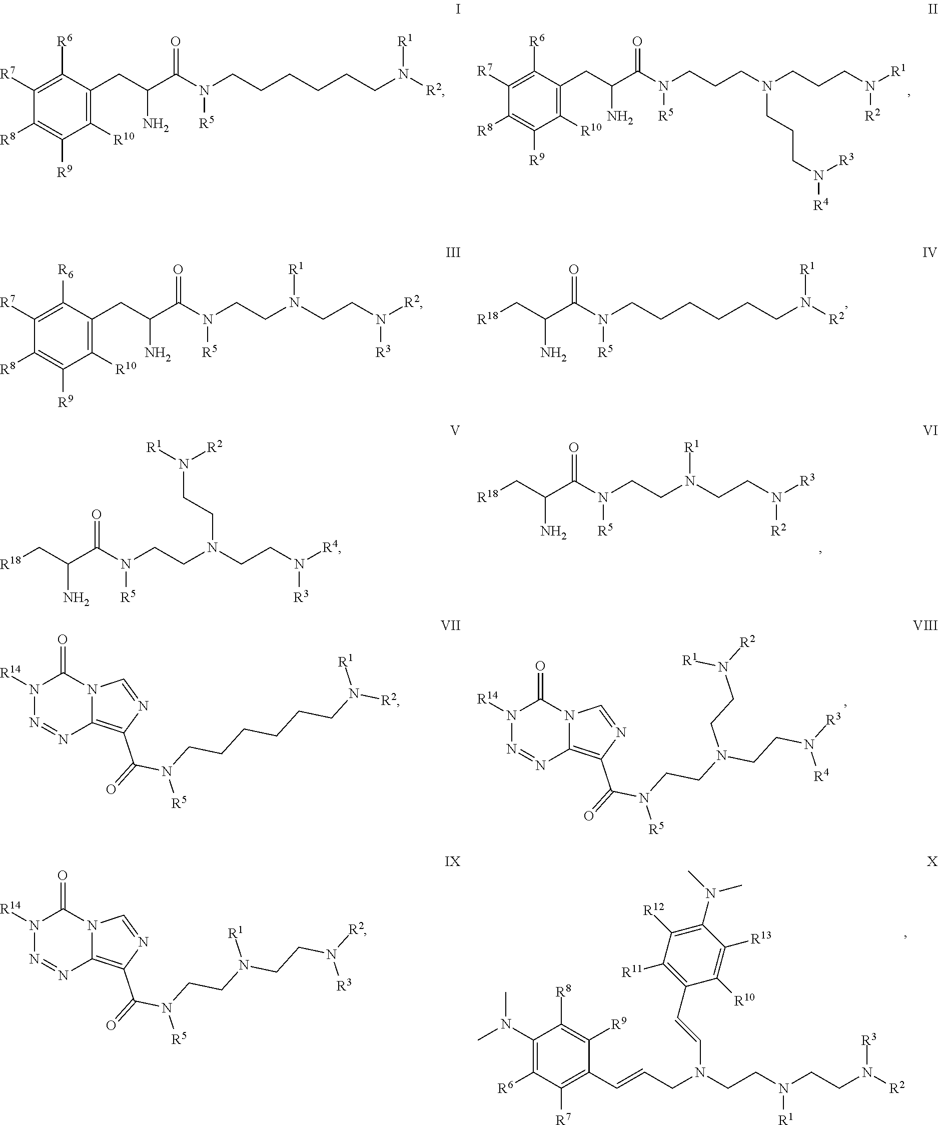

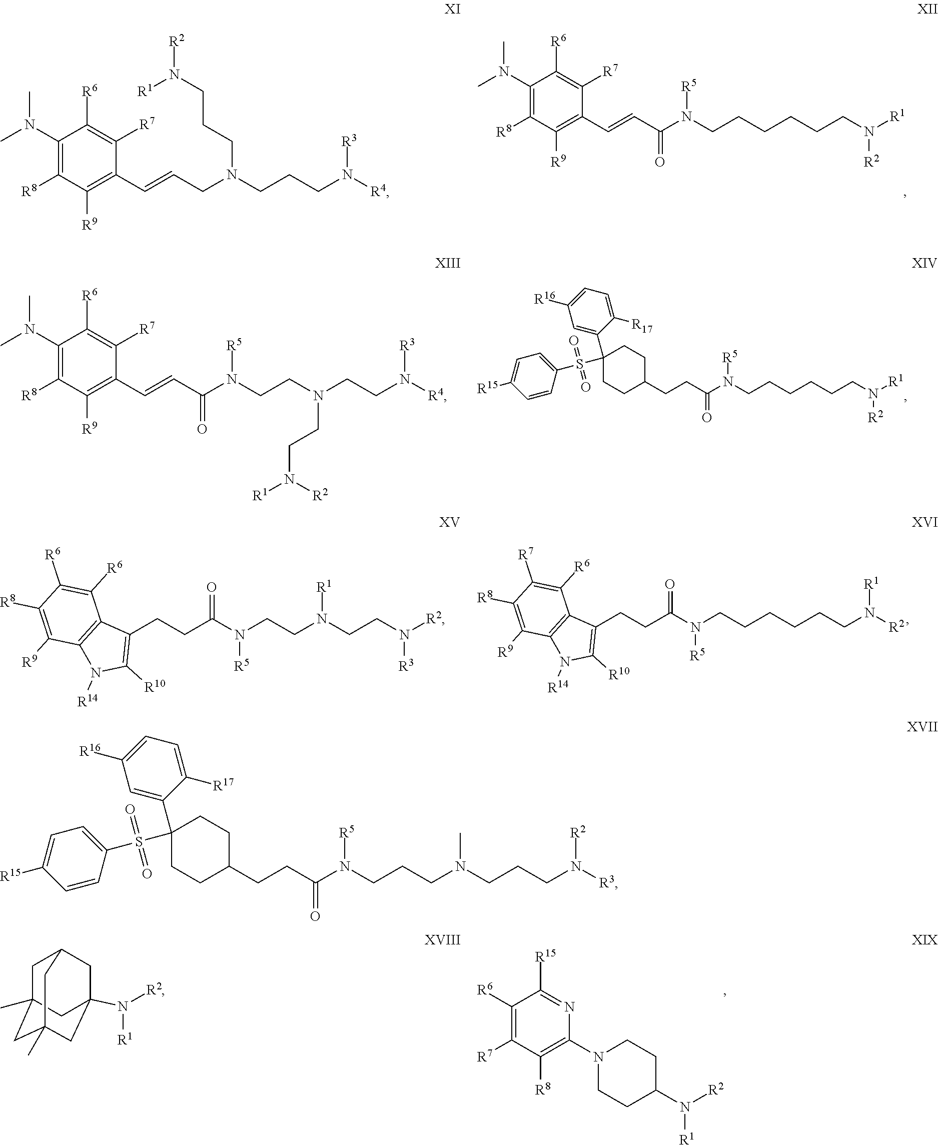

wherein
R¹, R², R³, and R⁴ are each independently a substituted or unsubstituted C₁-C₂₀ alkyl;
R⁵ and R¹⁴ are each independently hydrogen, a substituted or unsubstituted C₁-C₅ alkyl;
R⁶, R⁷, R⁸, R⁹, R¹⁰, R¹¹, R¹², and R¹³ are each independently hydrogen, OH, halogen, or substituted or unsubstituted C₁-C₅ alkyl;
R¹⁵, R¹⁶, and R¹⁷ are each independently hydrogen, OH, halogen, or substituted or unsubstituted C₁-C₅ alkyl; and
R¹⁸ is OH or substituted or unsubstituted C₁-C₁₀ alkyl.

In some aspects, the compound comprises a compound defined by any one of Formulas I-III, or a pharmaceutically acceptable salt thereof:

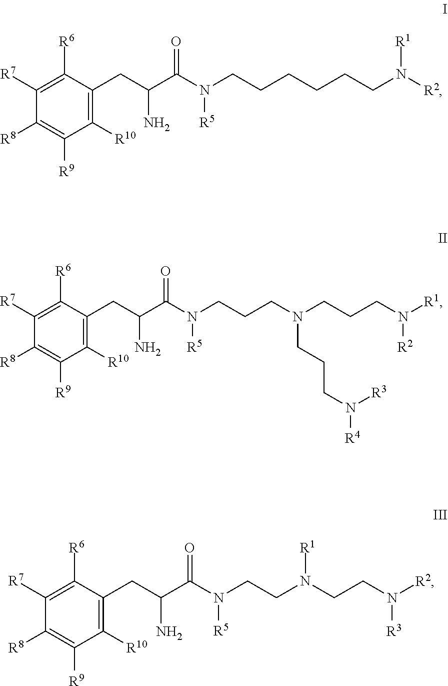

wherein
R¹, R², R³, and R⁴ are each independently a substituted or unsubstituted C₁-C₂₀ alkyl;
R⁵ is hydrogen, a substituted or unsubstituted C₁-C₅ alkyl; and
R⁶, R⁷, R⁸, R⁹, R¹⁰ are each independently hydrogen, OH, halogen, or substituted or unsubstituted C₁-C₅ alkyl.

In some aspects, the compound comprises a compound defined by Formulas IV-VI, or a pharmaceutically acceptable salt thereof:

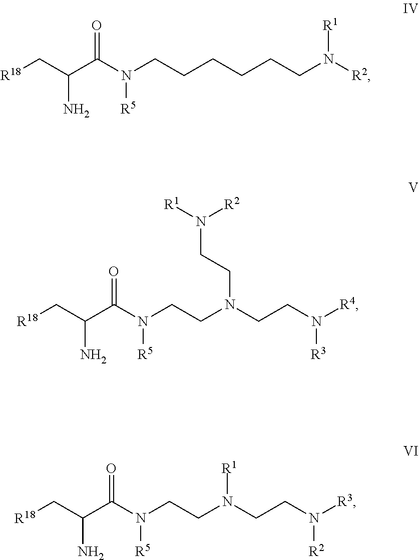

wherein
R¹, R², R³, and R⁴ are each independently a substituted or unsubstituted C₁-C₂₀ alkyl;
R⁵ is hydrogen, a substituted or unsubstituted C₁-C₅ alkyl; and
R¹⁸ is OH or substituted or unsubstituted C₁-C₁₀ alkyl.

In one aspect, the compound comprises a compound defined by Formulas VII-IX, or a pharmaceutically acceptable salt thereof:

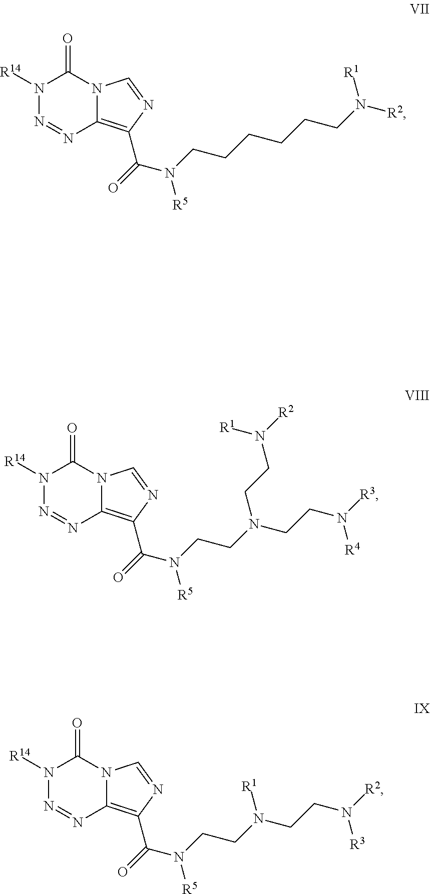

wherein
R¹, R², R³, and R⁴ are each independently a substituted or unsubstituted C₁-C₂₀ alkyl; and
R⁵ and R¹⁴ are each independently hydrogen, a substituted or unsubstituted C₁-C₅ alkyl.

In another aspect, the compound comprises a compound defined by Formulas X-XIII, or a pharmaceutically acceptable salt thereof:

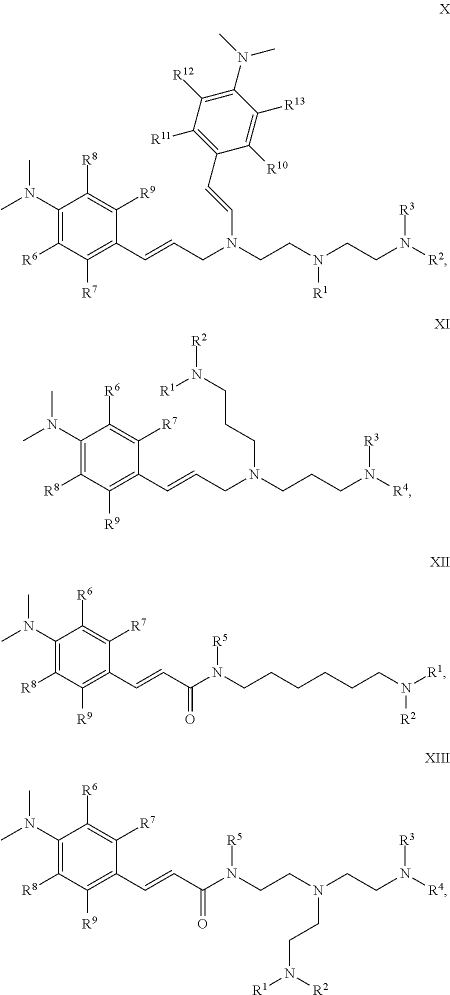

wherein
R¹, R², R³, and R⁴ are each independently a substituted or unsubstituted C₁-C₂₀ alkyl;
R⁵ is hydrogen, a substituted or unsubstituted C₁-C₅ alkyl; and
R⁶, R⁷, R⁸, R⁹, R¹⁰, R¹¹, R¹², and R¹³ are each independently hydrogen, OH, halogen, or substituted or unsubstituted C₁-C₅ alkyl.

In another aspect, the compound comprises a compound defined by Formulas XIV or XVII, or a pharmaceutically acceptable salt thereof:

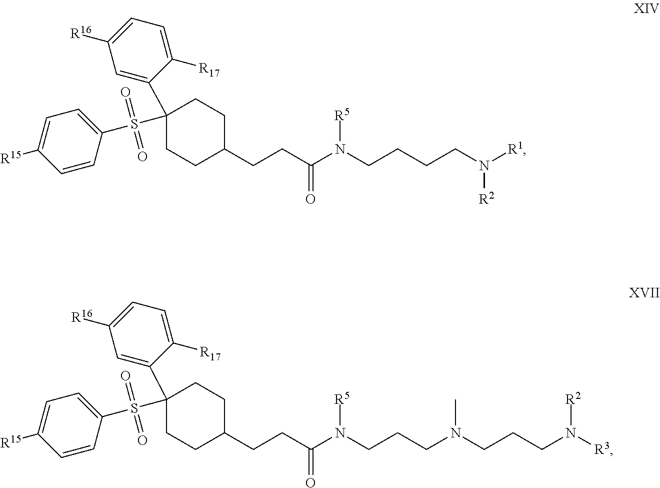

wherein
R¹, R², R³ are each independently a substituted or unsubstituted C₁-C₂₀ alkyl;
R⁵ is hydrogen, a substituted or unsubstituted C₁-C₅ alkyl;
R¹⁵, R¹⁶, and R¹⁷ are each independently hydrogen, OH, halogen, or substituted or unsubstituted C₁-C₅ alkyl.

In another aspect, the compound comprises a compound defined by Formulas XV-XVI, or a pharmaceutically acceptable salt thereof:

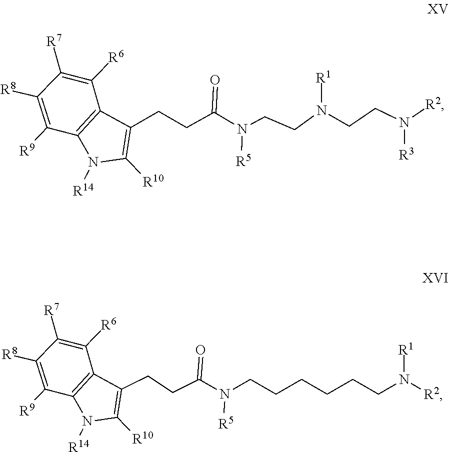

wherein
R¹, R², R³ are each independently a substituted or unsubstituted C₁-C₂₀ alkyl;
R⁵ and R¹⁴ are each independently hydrogen, a substituted or unsubstituted C₁-C₅ alkyl; and
R⁶, R⁷, R⁸, R⁹, and R¹⁰ are each independently hydrogen, OH, halogen, or substituted or unsubstituted C₁-C₅ alkyl.

In another aspect, the compound comprises a compound defined by Formula XVIII, or a pharmaceutically acceptable salt thereof:

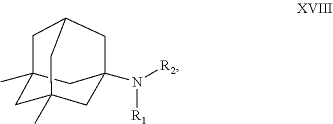

wherein
R¹ and R² are each independently a substituted or unsubstituted C₁-C₂₀ alkyl.

In another aspect, the compound comprises a compound defined by Formula XIX, or a pharmaceutically acceptable salt thereof:

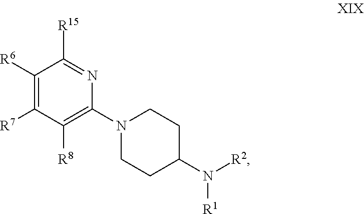

wherein
R¹ and R² are each independently a substituted or unsubstituted C₁-C₂₀ alkyl;
R⁶, R⁷, R⁸ are each independently hydrogen, OH, halogen, or substituted or unsubstituted C₁-C₅ alkyl; and
R¹⁵ is hydrogen, OH, halogen, or substituted or unsubstituted C₁-C₅ alkyl.

In some embodiments, the compound comprises a compound defined by any one of Formulas I-A through XIX-A, or a pharmaceutically acceptable salt thereof:

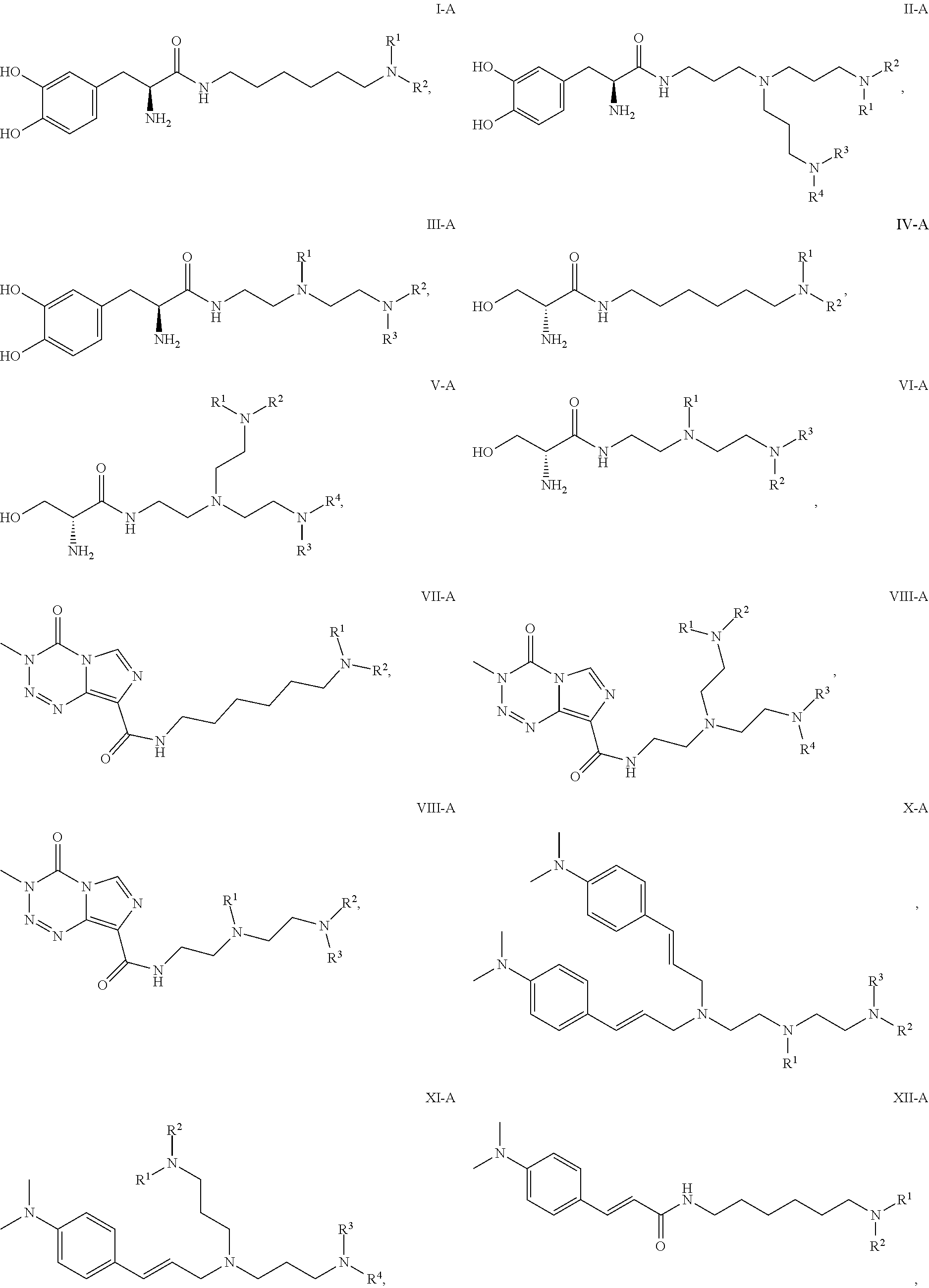

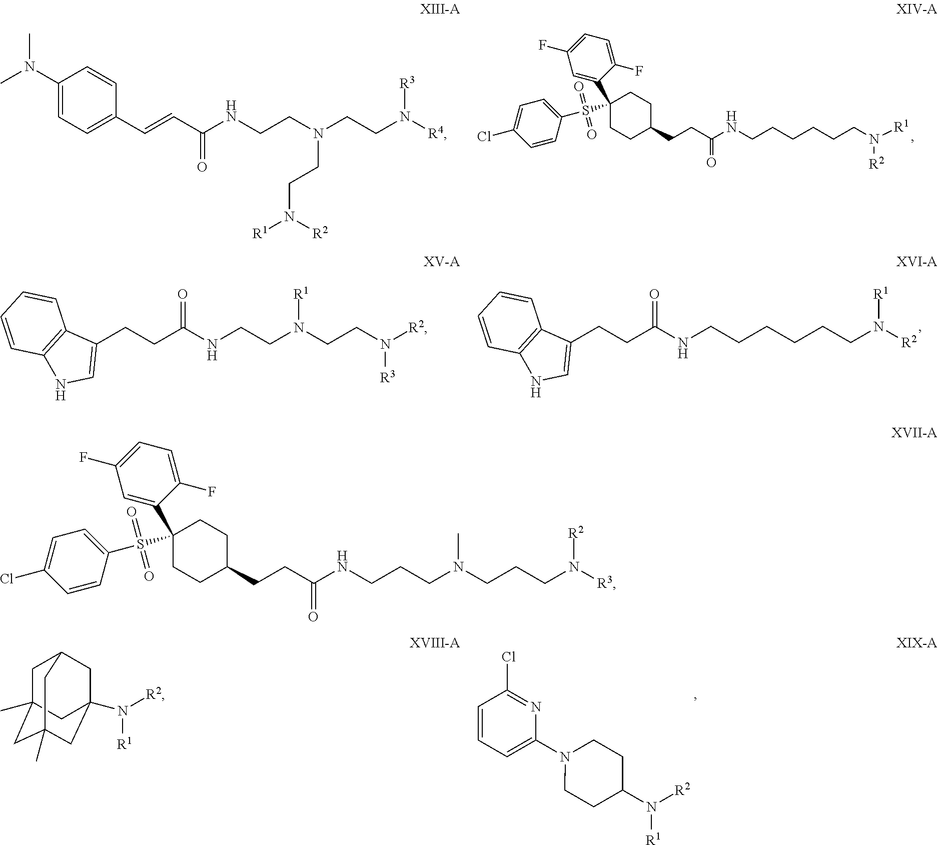

wherein
R¹, R², R³, and R⁴ are each independently a substituted or unsubstituted C₁-C₂₀ alkyl.

In some embodiments of Formulae I-XIX and IA-XIX-A described above, R¹, R², R³, and R⁴ may each independently represent a substituted or unsubstituted C₁-C₂₀ alkyl, such as a substituted or unsubstituted C₂-C₁₈, a substituted or unsubstituted C₄-C₁₆, a substituted or unsubstituted C₅-C₁₅, a substituted or unsubstituted C₅-C₁₂, a substituted or unsubstituted C₈-C₁₂, or a substituted or unsubstituted C₉-C₁₁. In some aspects, R¹, R², R³, and R⁴ may each independently be a substituted or unsubstituted C₅-C₁₅ alkyl. In some aspects, R¹, R², R³, and R⁴ are each independently a linear or branched unsubstituted alkyl. In other aspects, R¹, R², R³, and R⁴ are each independently a linear or branched substituted alkyl. R¹, R², R³, and R⁴, for example, may each independently be a mix of linear and branched substituted alkyl. In additional embodiments, R¹, R², R³, and R⁴ are each independently a mixture of linear or branched unsubstituted alkyl.

In some embodiments of Formulae I-XIX and IA-XIX-A described above, R¹, R², R³, and R⁴ are each independently a linear or branched C₁-C₁₅ alkyl (e.g. C₂-C₁₅, C₃-C₁₅, C₄-C₁₅, C₅-C₁₅, C₆-C₁₅, C₇-C₁₅, C₈-C₁₅, C₉-C₁₅, C₁₀-C₁₅) substituted with one or more substituents selected from the group consisting of ester, ether, carbonate ester, acetal, ketal, thioketal, thiol, sulfide, disulfide, diols, aryl, halogen, nitro, oxo, and amides. In some embodiments, R¹, R², R³, and R⁴ are each independently a linear or branched C₅-C₁₅ alkyl substituted with one or more substituents selected from the group consisting of ester, ether, carbonate ester, acetal, ketal, thioketal, thiol, sulfide, disulfide, diols, oxo, and amides. In some particular aspects, R¹, R², R³, and R⁴ are each independently a linear or branched C₅-C₁₅ alkyl substituted with one or more substituents selected from the group consisting of ester, ether, carbonate ester, sulfide, disulfide, diols, and amides.

In some embodiments of Formulae I-XIX and IA-XIX-A described above, R¹, R², R³, and R⁴ are each independently selected from the group consisting of:

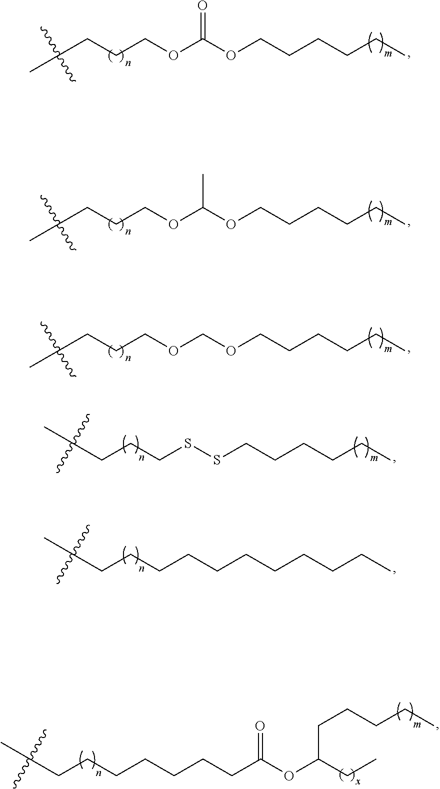

wherein N, M, and X each independently represent integers from 1 to 9, such as from 1 to 5.

In some embodiments of Formulae I-XIX and IA-MI-A described above, R¹, R², R³, and R⁴ are each independently selected from the group consisting of:

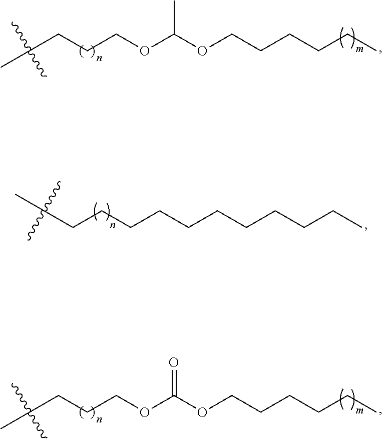

wherein N and M each independently represent integers from 1 to 9, such as from 1 to 5. In further aspects, at least one of R¹, R², R³, and R⁴ comprises:

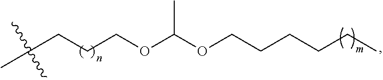

wherein N and M each independently represent integers from 1 to 9, such as from 1 to 5.

In even further aspects, each of R¹, R², R³, and R⁴ comprises:

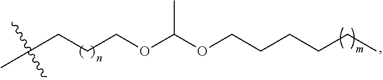

wherein N and M each independently represent integers from 1 to 9, such as from 1 to 5.

In some embodiments of Formulae I-XIX and IA-XIX-A described above, R¹, R², R³, and R⁴ are each independently selected from the group consisting of:

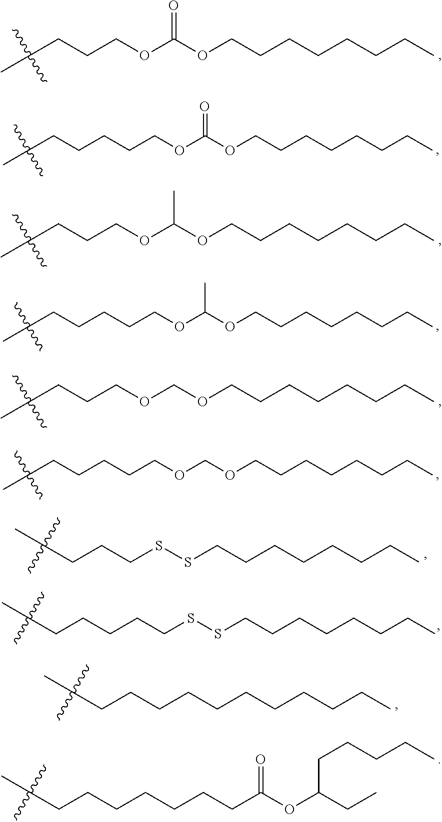

In certain aspects of Formulae I-XIX and IA-XIX-A described above, R¹, R², R³, and R⁴ are different. R¹, R², R³, and R⁴ are considered different if at least one of R¹, R², R³, and R⁴ is not the same as the remaining positions. For example, R¹ could be one moiety, while R², R³, and R⁴ are each independently different moieties from R¹. In some aspects, each of R¹, R², R³, and R⁴ are different. In other embodiments, R¹, R², R³, and R⁴ are all the same.

Although the application references R¹, R², R³, R⁴, R³, R⁶, R⁷, R⁸, R⁹, R¹⁰, R¹¹, R¹², R¹³, R¹⁴, R¹⁵, R¹⁶, R¹⁷, and R¹⁸ as broadly applied to a group of formulas, claim interpretation should only consider those R-groups which contain a corresponding number in the formula. For example, a claim including a group of formulas, e.g., Formula IV and Formula V:

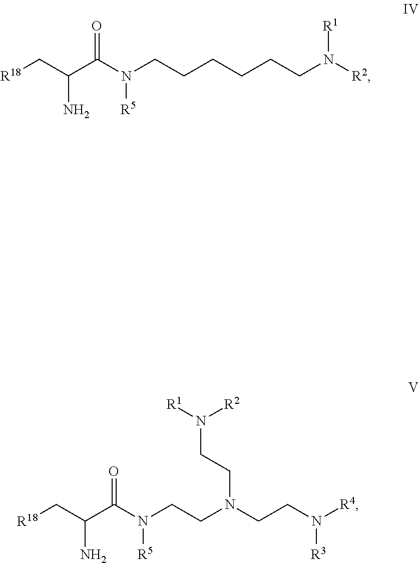

wherein R¹, R², R³, and R⁴ are each independently a substituted or unsubstituted C₁-C₂₀ alkyl; R⁵ is a substituted or unsubstituted C₁-C₅ alkyl; and R¹⁸ is OH or substituted or unsubstituted C₁-C₁₀ alkyl; should be interpreted such that the additional inclusion of R³ and R⁴ applies only to Formula V. Thus, where only R¹ and R² are depicted in a formula, the added reference to R³ and R⁴ would not apply.

In some aspects the compound comprises a compound defined by one of the structures depicted in BL1-BL83 in Table 1. For example the compound may be defined by any one of BL1; BL2; BL3; BL4; BL5; BL6; BL7; BL8; BL9; BL10; BL11; BL12; BL13; BL14; BL15; BL16; BL17; BL18; BL19; BL20; BL21; BL22; BL23; BL24; BL25; BL26; BL27; BL28; BL29; BL30; BL31; BL32; BL33; BL34; BL35; BL36; BL37; BL38; BL39; BL40; BL41; BL42; BL43; BL44; BL45; BL46; BL47; BL48; BL49; BL50; BL51; BL52; BL53; BL54; BL55; BL56; BL57; BL58; BL59; BL60; BL61; BL62; BL63; BL64, BL65; BL66; BL67; BL68; BL69; BL70; BL71; BL72; BL73; BL74; BL75; BL76; BL77; BL78; BL79; BL80; BL81; BL82; or BL83, shown in Table 1. For example, the compound can be defined by one or more of BL6; BL7; BL8; BL28; BL38; BL39; BL41; BL43; BL53; BL54; BL55; BL56; BL57; BL62; BL65; BL67; BL68; BL; 69 BL70. In some aspects, the compound is defined by one or more of BL8; BL28; BL39; BL41; BL54; BL56; BL62; BL65; BL68; BL; 69; or BL70. In certain aspects, the compound is defined by one or more of the group consisting of BL28; BL39; BL54; BL56; BL68; and BL70.

In a particular aspect, the compound is defined by BL28:

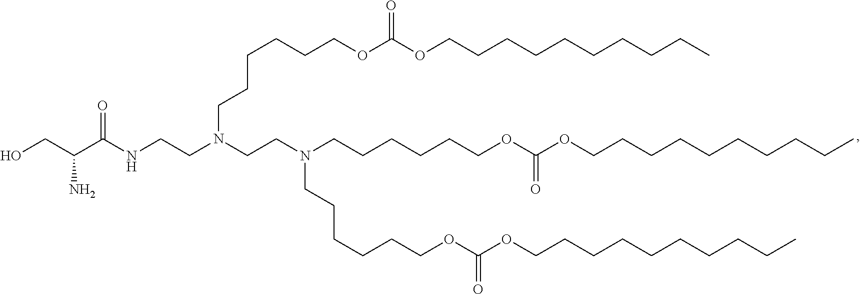 [SMILES: CCCCCCCCCCOC(=O)OCCCCCCN(CCCCCCOC(=O)OCCCCCCCCCC)CCN(CCCCCCOC(=O)OCCCCCCCCCC)CCNC(=O)[C@H](N)CO]

or a pharmaceutically acceptable salt thereof.

In other aspects, the compound is defined by BL39:

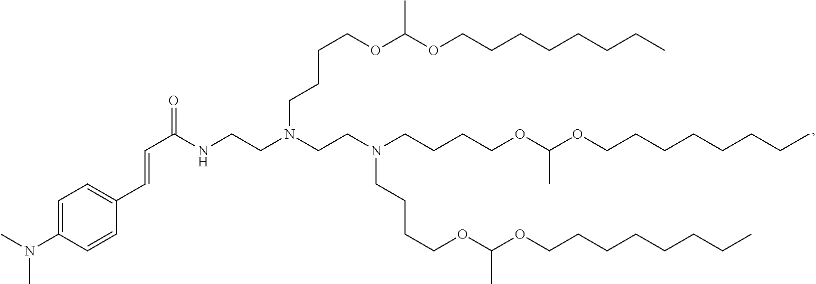 [SMILES: CCCCCCCCOC(C)OCCCCN(CCCCOC(C)OCCCCCCCC)CCN(CCCCOC(C)OCCCCCCCC)CCNC(=O)/C=C/c1ccc(N(C)C)cc1]

or a pharmaceutically acceptable salt thereof.

In other aspects the compound is defined by BL54:

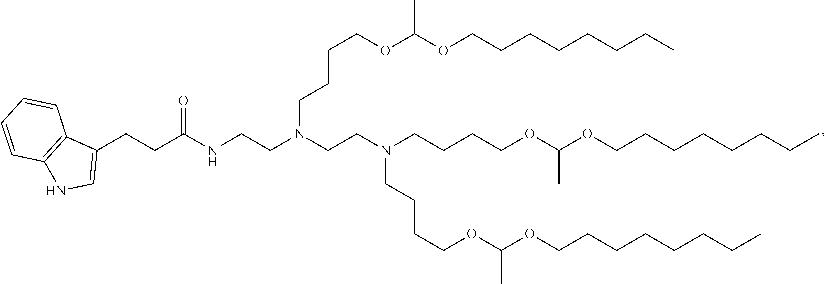 [SMILES: CCCCCCCCOC(C)OCCCCN(CCCCOC(C)OCCCCCCCC)CCN(CCCCOC(C)OCCCCCCCC)CCNC(=O)CCc1c[nH]c2ccccc12]

or a pharmaceutically acceptable salt thereof.

In some other aspects, the compound is defined by BL56:

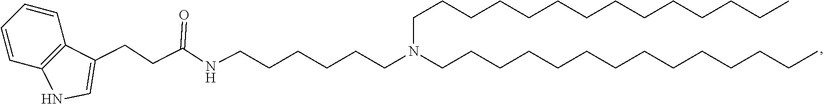 [SMILES: CCCCCCCCCCCCCCN(CCCCCCCCCCCCCC)CCCCCCNC(=O)CCc1c[nH]c2ccccc12]

or a pharmaceutically acceptable salt thereof.

In another aspect, the compound is defined by BL68:

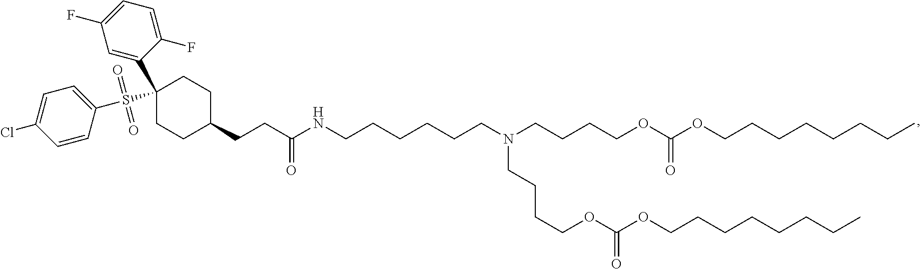 [SMILES: CCCCCCCCOC(=O)OCCCCN(CCCCCCNC(=O)CC[C@H]1CC[C@](c2cc(F)ccc2F)(S(=O)(=O)c2ccc(Cl)cc2)CC1)CCCCOC(=O)OCCCCCCCC]

or a pharmaceutically acceptable salt thereof.

In other aspects, the compound is defined by BL70:

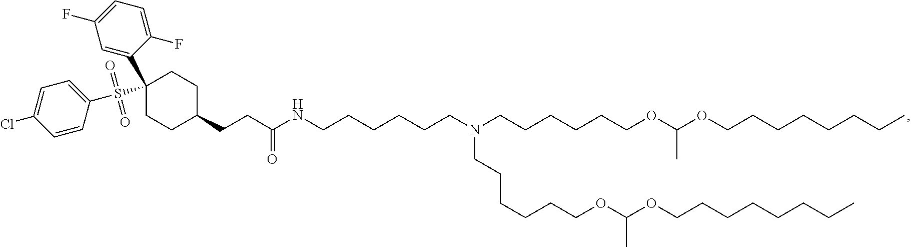 [SMILES: CCCCCCCCOC(C)OCCCCCCN(CCCCCCNC(=O)CC[C@H]1CC[C@](c2cc(F)ccc2F)(S(=O)(=O)c2ccc(Cl)cc2)CC1)CCCCCCOC(C)OCCCCCCCC]

or a pharmaceutically acceptable salt thereof.

### Lipid Particles and Methods of Use

Disclosed herein are lipid nanomaterials for gene therapy and drug delivery applications. As the name suggests, “nanomaterials” have sizes ranging in the nanometer scale. For example, the term “nanoparticle” as used herein refer to particles having a diameter of at least 1 nm, at least 10 nm, at least 50 nm, at least 80 nm, at least 100 nm, at least 120 nm, at least 150 nm, at least 160 nm, at least 170 nm, at least 180 nm, at least 190 nm, at least 200 nm, at least 210 nm, at least 220 nm, at least 230 nm, at least 240 nm, at least 250 nm, at least 300 nm, at least 400 nm, at least 500 nm, at least 600 nm, at least 700 nm, at least 800 nm, at least 900 nm, or at least 1000 nm. In some embodiments, the nanoparticles may have a diameter of less than 1,000 nm, less than 900 nm, less than 800 nm, less than 700 nm, less than 600 nm, less than 500 nm, less than 450 nm, less than 400 nm, less than 350 nm, less than 320 nm, less than 300 nm, less than 280 nm, less than 250 nm, less than 200 nm, less than 190 nm, less than 180 nm, less than 170 nm, less than 160 nm, or less than 150 nm. The diameter of nanoparticles can range from any of the minimum values described above to any of the maximum values described above, for example from 1 nm to 1,000 nm, from 50 nm to 500 nm, from 100 nm to 350 nm, from 100 nm to 300 nm, from 100 nm to 250 nm, or from 100 nm to 200 nm. This list is intended to be merely for purposes of example only, and any of numerous combinations of minimum and maximum values described above may be used as a range of nanoparticle diameters in a vehicle.

Production of the nanoparticles can result in bimodal or multi-modal distributions of diameters. Desired diameter ranges (e.g. a monodispersed diameter range) can be separated by centrifugation and collected. Monodispersed diameter ranges can be analyzed and counted using several methods including, for example, dynamic light scattering and nanoparticle tracking analysis. In some embodiments, the nanoparticles have a monodispersed average diameter distribution, such as from 100 nm to 200 nm. As used herein, a monodispersed (or homogenous) average diameter distribution refers to a population of nanoparticles wherein greater than 80 percent of the population vary in diameter by about 20 percent or less from the average diameter. In some examples, the monodispersed (or homogenous) average diameter distribution refers to a population of nanoparticles having an average polydispersity index of 0.3 or less, 0.25 or less, 0.2 or less, from 0.1 to 0.3 or from 0.15 to 0.25.

In some examples, the lipid particle can further comprise an additional component, such as an additional lipid. In some examples, the additional lipid can comprise a phospholipid, a sterol, or a combination thereof. In some examples, the lipid particle can further comprise 1,2-dioleoyl-sn-glycero-3-phosphoethanolamine (DOPE), cholesterol, 1,2-dimyristoyl-rac-glycero-3-methylpolyoxyethylene, or a combination thereof.

As described herein, the lipid nanomaterials disclosed herein comprise a lipid. In general, lipids include fats and fat-derived materials that are relatively insoluble in water but soluble in organic solvents, are related either actually or potentially to fatty acid esters, fatty alcohols, sterols, waxes, or the like, and are utilizable by the animal organism. Lipids are one of the chief structural components of living cells. As an example, fats are glyceryl esters of higher fatty acids.

In certain embodiments, the disclosure relates to methods of treating or preventing a disease or disorder of the brain comprising administering an effective amount of a composition comprising the lipid nanoparticles disclosed herein to a subject in need thereof.

Formulations containing one or more of the compounds described herein can be prepared using a pharmaceutically acceptable carrier composed of materials that are considered safe and effective and can be administered to an individual without causing undesirable biological side effects or unwanted interactions. The carrier is all components present in the pharmaceutical formulation other than the active ingredient or ingredients. As generally used herein “carrier” includes, but is not limited to, diluents, binders, lubricants, disintegrators, fillers, pH modifying agents, preservatives, antioxidants, solubility enhancers, and coating compositions.

Optional pharmaceutically acceptable excipients present in the drug-containing tablets, beads, granules or particles include, but are not limited to, diluents, binders, lubricants, disintegrants, colorants, stabilizers, and surfactants. Diluents, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

Stabilizers are used to inhibit or retard drug decomposition reactions which include, by way of example, oxidative reactions.

The lipid nanomaterials described herein can be administered adjunctively with other active compounds. These additional active compounds include but are not limited to analgesics, anti-inflammatory drugs, antipyretics, antidepressants, antiepileptics, antihistamines, antimigraine drugs, antimuscarinics, anxioltyics, sedatives, hypnotics, antipsychotics, bronchodilators, anti-asthma drugs, cardiovascular drugs, corticosteroids, dopaminergics, electrolytes, gastro-intestinal drugs, muscle relaxants, nutritional agents, vitamins, parasympathomimetics, stimulants, anorectics, anti-narcoleptics, and antiviral agents. “Adjunctive administration”, as used herein, means the compound can be administered in the same dosage form or in separate dosage forms with one or more other active agents. The additional active agent(s) can be formulated for immediate release, controlled release, or combinations thereof.

In another aspect, the present invention provides methods of treating and/or preventing a disease, such as a genetic disease, a proliferative disease, a hematological disease, a neurological disease, an immunological disease, a gastrointestinal disease (e.g., liver disease), a respiratory disease (e.g., lung disease), a painful condition, a psychiatric disorder, a metabolic disorder, a musculoskeletal disorder, or a spleen disease in a subject in need thereof. In certain embodiments, the disease that is treated and/or prevented by the inventive methods is hepatic carcinoma, hypercholesterolemia, refractory anemia, or familial amyloid neuropathy. In certain embodiments, the method of treating and/or preventing a disease comprises administering a composition of the invention to the subject.

The term “genetic disease” refers to a disease caused by one or more abnormalities in the genome of a subject, such as a disease that is present from birth of the subject. Genetic diseases may be heritable and may be passed down from the parents' genes. A genetic disease may also be caused by mutations or changes of the DNAs and/or RNAs of the subject. In such cases, the genetic disease will be heritable if it occurs in the germline. Exemplary genetic diseases include, but are not limited to, Aarskog-Scott syndrome, Aase syndrome, achondroplasia, acrodysostosis, addiction, adreno-leukodystrophy, albinism, ablepharon-macrostomia syndrome, alagille syndrome, alkaptonuria, alpha-1 antitrypsin deficiency, Alport's syndrome, Alzheimer's disease, asthma, autoimmune polyglandular syndrome, androgen insensitivity syndrome, Angelman syndrome, ataxia, ataxia telangiectasia, atherosclerosis, attention deficit hyperactivity disorder (ADHD), autism, baldness, Batten disease, Beckwith-Wiedemann syndrome, Best disease, bipolar disorder, brachydactyl), breast cancer, Burkitt lymphoma, chronic myeloid leukemia, Charcot-Marie-Tooth disease, Crohn's disease, cleft lip, Cockayne syndrome, Coffin Lowry syndrome, colon cancer, congenital adrenal hyperplasia, Cornelia de Lange syndrome, Costello syndrome, Cowden syndrome, craniofrontonasal dysplasia, Crigler-Najjar syndrome, Creutzfeldt-Jakob disease, cystic fibrosis, deafness, depression, diabetes, diastrophic dysplasia, DiGeorge syndrome, Down's syndrome, dyslexia, Duchenne muscular dystrophy, Dubowitz syndrome, ectodermal dysplasia Ellis-van Creveld syndrome, Ehlers-Danlos, epidermolysis bullosa, epilepsy, essential tremor, familial hypercholesterolemia, familial Mediterranean fever, fragile X syndrome, Friedreich's ataxia, Gaucher disease, glaucoma, glucose galactose malabsorption, glutaricaciduria, gyrate atrophy, Goldberg Shprintzen syndrome (velocardiofacial syndrome), Gorlin syndrome, Hailey-Hailey disease, hemihypertrophy, hemochromatosis, hemophilia, hereditary motor and sensory neuropathy (HMSN), hereditary non polyposis colorectal cancer (HNPCC), Huntington's disease, immunodeficiency with hyper-IgM, juvenile onset diabetes, Klinefelter's syndrome, Kabuki syndrome, Leigh's disease, long QT syndrome, lung cancer, malignant melanoma, manic depression, Marfan syndrome, Menkes syndrome, miscarriage, mucopolysaccharide disease, multiple endocrine neoplasia, multiple sclerosis, muscular dystrophy, myotrophic lateral sclerosis, myotonic dystrophy, neurofibromatosis, Niemann-Pick disease, Noonan syndrome, obesity, ovarian cancer, pancreatic cancer, Parkinson's disease, paroxysmal nocturnal hemoglobinuria, Pendred syndrome, peroneal muscular atrophy, phenylketonuria (PKU), polycystic kidney disease, Prader-Willi syndrome, primary biliary cirrhosis, prostate cancer, REAR syndrome, Refsum disease, retinitis pigmentosa, retinoblastoma, Rett syndrome, Sanfilippo syndrome, schizophrenia, severe combined immunodeficiency, sickle cell anemia, spina bifida, spinal muscular atrophy, spinocerebellar atrophy, sudden adult death syndrome, Tangier disease, Tay-Sachs disease, thrombocytopenia absent radius syndrome, Townes-Brocks syndrome, tuberous sclerosis, Turner syndrome, Usher syndrome, von Hippel-Lindau syndrome, Waardenburg syndrome, Weaver syndrome, Werner syndrome, Williams syndrome, Wilson's disease, xeroderma piginentosum, and Zellweger syndrome.

A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, angiogenesis, inflammatory diseases, and autoimmune diseases.

The term “angiogenesis” refers to the physiological process through which new blood vessels form from pre-existing vessels. Angiogenesis is distinct from vasculogenesis, which is the de novo formation of endothelial cells from mesoderm cell precursors. The first vessels in a developing embryo form through vasculogenesis, after which angiogenesis is responsible for most blood vessel growth during normal or abnormal development. Angiogenesis is a vital process in growth and development, as well as in wound healing and in the formation of granulation tissue. However, angiogenesis is also a fundamental step in the transition of tumors from a benign state to a malignant one, leading to the use of angiogenesis inhibitors in the treatment of cancer. Angiogenesis may be chemically stimulated by angiogenic proteins, such as growth factors (e.g., VEGF). “Pathological angiogenesis” refers to abnormal (e.g., excessive or insufficient) angiogenesis that amounts to and/or is associated with a disease.

The terms “neoplasm” and “tumor” are used interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

The term “cancer” refers to a malignant neoplasm (Stedman's Medical Dictionary, 25th ed.; Hensyl ed.; Williams & Wilkins: Philadelphia, 1990). Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendocrinetumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic adenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

The term “inflammatory disease” refers to a disease caused by, resulting from, or resulting in inflammation. The term “inflammatory disease” may also refer to a dysregulated inflammatory reaction that causes an exaggerated response by macrophages, granulocytes, and/or T-lymphocytes leading to abnormal tissue damage and/or cell death. An inflammatory disease can be either an acute or chronic inflammatory condition and can result from infections or non-infectious causes. Inflammatory diseases include, without limitation, atherosclerosis, arteriosclerosis, autoimmune disorders, multiple sclerosis, systemic lupus erythematosus, polymyalgia rheumatica (PMR), gouty arthritis, degenerative arthritis, tendonitis, bursitis, psoriasis, cystic fibrosis, arthrosteitis, rheumatoid arthritis, inflammatory arthritis, Sjogren's syndrome, giant cell arteritis, progressive systemic sclerosis (scleroderma), ankylosing spondylitis, polymyositis, dermatomyositis, pemphigus, pemphigoid, diabetes (e.g., Type I), myasthenia gravis, Hashimoto's thyroiditis, Graves' disease, Goodpasture's disease, mixed connective tissue disease, sclerosing cholangitis, inflammatory bowel disease, Crohn's disease, ulcerative colitis, pernicious anemia, inflammatory dermatoses, usual interstitial pneumonitis (UIP), asbestosis, silicosis, bronchiectasis, berylliosis, talcosis, pneumoconiosis, sarcoidosis, desquamative interstitial pneumonia, lymphoid interstitial pneumonia, giant cell interstitial pneumonia, cellular interstitial pneumonia, extrinsic allergic alveolitis, Wegener's granulomatosis and related forms of angiitis (temporal arteritis and polyarteritis nodosa), inflammatory dermatoses, hepatitis, delayed-type hypersensitivity reactions (e.g., poison ivy dermatitis), pneumonia, respiratory tract inflammation, Adult Respiratory Distress Syndrome (ARDS), encephalitis, immediate hypersensitivity reactions, asthma, hayfever, allergies, acute anaphylaxis, rheumatic fever, glomerulonephritis, pyelonephritis, cellulitis, cystitis, chronic cholecystitis, ischemia (ischemic injury), reperfusion injury, allograft rejection, host-versus-graft rejection, appendicitis, arteritis, blepharitis, bronchiolitis, bronchitis, cervicitis, cholangitis, chorioamnionitis, conjunctivitis, dacryoadenitis, dermatomyositis, endocarditis, endometritis, enteritis, enterocolitis, epicondylitis, epididymitis, fasciitis, fibrositis, gastritis, gastroenteritis, gingivitis, ileitis, iritis, laryngitis, myelitis, myocarditis, nephritis, omphalitis, oophoritis, orchitis, osteitis, otitis, pancreatitis, parotitis, pericarditis, pharyngitis, pleuritis, phlebitis, pneumonitis, proctitis, prostatitis, rhinitis, salpingitis, sinusitis, stomatitis, synovitis, testitis, tonsillitis, urethritis, urocystitis, uveitis, vaginitis, vasculitis, vulvitis, vulvovaginitis, angitis, chronic bronchitis, osteomyelitis, optic neuritis, temporal arteritis, transverse myelitis, necrotizing fasciitis, and necrotizing enterocolitis. An ocular inflammatory disease includes, but is not limited to, post-surgical inflammation.

An “autoimmune disease” refers to a disease arising from an inappropriate immune response of the body of a subject against substances and tissues normally present in the body. In other words, the immune system mistakes some part of the body as a pathogen and attacks its own cells. This may be restricted to certain organs (e.g., in autoimmune thyroiditis) or involve a particular tissue in different places (e.g., Goodpasture's disease which may affect the basement membrane in both the lung and kidney). The treatment of autoimmune diseases is typically with immunosuppression, e.g., medications which decrease the immune response. Exemplary autoimmune diseases include, but are not limited to, glomerulonephritis, Goodpasture's syndrome, necrotizing vasculitis, lymphadenitis, peri-arteritis nodosa, systemic lupus erythematosis, rheumatoid arthritis, psoriatic arthritis, systemic lupus erythematosis, psoriasis, ulcerative colitis, systemic sclerosis, dermatomyositis/polymyositis, anti-phospholipid antibody syndrome, scleroderma, pemphigus vulgaris, ANCA-associated vasculitis (e.g., Wegener's granulomatosis, microscopic polyangiitis), uveitis, Sjogren's syndrome, Crohn's disease, Reiter's syndrome, ankylosing spondylitis, Lyme disease, Guillain-Barre syndrome, Hashimoto's thyroiditis, and cardiomyopathy.

The term “liver disease” or “hepatic disease” refers to damage to or a disease of the liver. Non-limiting examples of liver disease include intrahepatic cholestasis (e.g., alagille syndrome, biliary liver cirrhosis), fatty liver (e.g., alcoholic fatty liver, Reye's syndrome), hepatic vein thrombosis, hepatolenticular degeneration (i.e., Wilson's disease), hepatomegaly, liver abscess (e.g., amebic liver abscess), liver cirrhosis (e.g., alcoholic, biliary, and experimental liver cirrhosis), alcoholic liver diseases (e.g., fatty liver, hepatitis, cirrhosis), parasitic liver disease (e.g., hepatic echinococcosis, fascioliasis, amebic liver abscess), jaundice (e.g., hemolytic, hepatocellular, cholestatic jaundice), cholestasis, portal hypertension, liver enlargement, ascites, hepatitis (e.g., alcoholic hepatitis, animal hepatitis, chronic hepatitis (e.g., autoimmune, hepatitis B, hepatitis C, hepatitis D, drug induced chronic hepatitis), toxic hepatitis, viral human hepatitis (e.g., hepatitis A, hepatitis B, hepatitis C, hepatitis D, hepatitis E), granulomatous hepatitis, secondary biliary cirrhosis, hepatic encephalopathy, varices, primary biliary cirrhosis, primary sclerosing cholangitis, hepatocellular adenoma, hemangiomas, bile stones, liver failure (e.g., hepatic encephalopathy, acute liver failure), angiomyolipoma, calcified liver metastases, cystic liver metastases, fibrolamellar hepatocarcinoma, hepatic adenoma, hepatoma, hepatic cysts (e.g., Simple cysts, Polycystic liver disease, hepatobiliary cystadenoma, choledochal cyst), mesenchymal tumors (mesenchymal hamartoma, infantile hemangioendothelioma, hemangioma, peliosis hepatis, lipomas, inflammatory pseudotumor), epithelial tumors (e.g., bile duct hamartoma, bile duct adenoma), focal nodular hyperplasia, nodular regenerative hyperplasia, hepatoblastoma, hepatocellular carcinoma, cholangiocarcinoma, cystadenocarcinoma, tumors of blood vessels, angiosarcoma, Karposi's sarcoma, hemangioendothelioma, embryonal sarcoma, fibrosarcoma, leiomyosarcoma, rhabdomyosarcoma, carcinosarcoma, teratoma, carcinoid, squamous carcinoma, primary lymphoma, peliosis hepatis, erythrohepatic porphyria, hepatic porphyria (e.g., acute intermittent Porphyria, Porphyria cutanea tarda), and Zellweger syndrome.

The term “spleen disease” refers to a disease of the spleen. Example of spleen diseases include, but are not limited to, splenomegaly, spleen cancer, asplenia, spleen trauma, idiopathic purpura, Felty's syndrome, Hodgkin's disease, and immune-mediated destruction of the spleen.

The term “lung disease” or “pulmonary disease” refers to a disease of the lung. Examples of lung diseases include, but are not limited to, bronchiectasis, bronchitis, bronchopulmonary dysplasia, interstitial lung disease, occupational lung disease, emphysema, cystic fibrosis, acute respiratory distress syndrome (ARDS), severe acute respiratory syndrome (SARS), asthma (e.g., intermittent asthma, mild persistent asthma, moderate persistent asthma, severe persistent asthma), chronic bronchitis, chronic obstructive pulmonary disease (COPD), emphysema, interstitial lung disease, sarcoidosis, asbestosis, aspergilloma, aspergillosis, pneumonia (e.g., lobar pneumonia, multilobar pneumonia, bronchial pneumonia, interstitial pneumonia), pulmonary fibrosis, pulmonary tuberculosis, rheumatoid lung disease, pulmonary embolism, and lung cancer (e.g., non-small-cell lung carcinoma (e.g., adenocarcinoma, squamous-cell lung carcinoma, large-cell lung carcinoma), small-cell lung carcinoma).

A “hematological disease” includes a disease which affects a hematopoietic cell or tissue. Hematological diseases include diseases associated with aberrant hematological content and/or function. Examples of hematological diseases include diseases resulting from bone marrow irradiation or chemotherapy treatments for cancer, diseases such as Pernicious Anemia, Hemorrhagic Anemia, Hemolytic Anemia, Aplastic Anemia, Sickle Cell Anemia, Sideroblastic Anemia, Anemia associated with chronic infections such as Malaria, Trypanosomiasis, HTV, Hepatitis virus or other viruses, Myelophthisic Anemias caused by marrow deficiencies, renal failure resulting from Anemia, Anemia, Polycethemia, Infectious Mononucleosis (EVI), Acute Non-Lymphocytic Leukemia (ANLL), Acute Myeloid Leukemia (AML), Acute Promyelocytic Leukemia (APL), Acute Myelomonocytic Leukemia (AMMoL), Polycethemia Vera, Lymphoma, Acute Lymphocytic Leukemia (ALL), Chronic Lymphocytic Leukemia, Wilm's Tumor, Ewing's Sarcoma, Retinoblastoma, Hemophilia, disorders associated with an increased risk of Thrombosis, Herpes, Thalessemia, antibody-mediated disorders such as transfusion reactions and Erythroblastosis, mechanical trauma to red blood cells such as micro-angiopathic hemolytic anemias, Thrombotic Thrombocytopenic Purpura and disseminated intravascular coagulation, infections by parasites such as Plasmodium, chemical injuries from, e.g., lead poisoning, and Hypersplenism.

The term “neurological disease” refers to any disease of the nervous system, including diseases that involve the central nervous system (brain, brainstem and cerebellum), the peripheral nervous system (including cranial nerves), and the autonomic nervous system (parts of which are located in both central and peripheral nervous system). Neurodegenerative diseases also refer to a type of neurological disease marked by the loss of nerve cells, including, but not limited to, Alzheimer's disease, Parkinson's disease, amyotrophic lateral sclerosis, tauopathies (including fronto-temporal dementia), and Huntington's disease. Examples of neurological diseases include, but are not limited to, headache, stupor and coma, dementia, seizure, sleep disorders, trauma, infections, neoplasms, neuroophthalmology, movement disorders, demyelinating diseases, spinal cord disorders, and disorders of peripheral nerves, muscle and neuromuscular junctions. Addiction and mental illness, include, but are not limited to, bipolar disorder and schizophrenia, are also included in the definition of neurological diseases. Further examples of neurological diseases include Acquired Epileptiform Aphasia; Acute Disseminated Encephalomyelitis; Adrenoleukodystrophy; Agenesis of the corpus callosum; Agnosia; Aicardi syndrome; Alexander disease: Alpers' disease; Alternating hemiplegia; Alzheimer's disease; Amyotrophic lateral sclerosis; Anencephaly; Angelman syndrome; Angiomatosis; Anoxia; Aphasia: Apraxia; Arachnoid Cysts; Arachnoiditis; Arnold-Chiari malformation; Arteriovenous malformation; Asperger syndrome; Ataxia Telangiectasia; Attention Deficit Hyperactivity Disorder; Autism; Autonomic Dysfunction; Back Pain; Batten disease; Behcet's disease; Bell's palsy; Benign Essential Blepharospasm; Benign Focal; Amyotrophy; Benign Intracranial Hypertension; Binswanger's disease; Blepharospasm; Bloch Sulzberger syndrome; Brachial plexus injury; Brain abscess; Brain injury; Brain tumors (including Glioblastoma multiforme); Spinal tumor; Brown-Sequard syndrome; Canavan disease; Carpal tunnel syndrome (CTS); Causalgia; Central pain syndrome; Central pontine myelinolysis; Cephalic disorder; Cerebral aneurysm; Cerebral arteriosclerosis; Cerebral atrophy; Cerebral gigantism; Cerebral palsy; Charcot-Marie-Tooth disease; Chemotherapy-induced neuropathy and neuropathic pain; Chiari malformation; Chorea; Chronic inflammatory demyelinating polyneuropathy (CIDP); Chronic pain; Chronic regional pain syndrome; Coffin Lowry syndrome; Coma, including Persistent Vegetative State; Congenital facial diplegia; Corticobasal degeneration; Cranial arteritis; Craniosynostosis; Creutzfeldt-Jakob disease; Cumulative trauma disorders; Cushing's syndrome; Cytomegalic inclusion body disease (CIBD); Cytomegalovirus Infection; Dancing eyes-dancing feet syndrome; Dandy-Walker syndrome; Dawson disease; De Morsier's syndrome; Dejerine-Klumpke palsy; Dementia; Dermatomyositis; Diabetic neuropathy; Diffuse sclerosis; Dysautonomia; Dysgraphia; Dyslexia; Dystonias; Early infantile epileptic encephalopathy; Empty sella syndrome; Encephalitis; Encephaloceles; Encephalotrigeminal angiomatosis; Epilepsy; Erb's palsy; Essential tremor; Fabry's disease; Fahr's syndrome; Fainting; Familial spastic paralysis; Febrile seizures; Fisher syndrome; Friedreich's ataxia; Fronto-Temporal Dementia and other “Tauopathies”; Gaucher's disease; Gerstmann's syndrome; Giant cell arteritis; Giant cell inclusion disease; Globoid cell Leukodystrophy; Guillain-Barre syndrome; HTLV-1 associated myelopathy; Hallervorden-Spatz disease; Head injury; Headache; Hemifacial Spasm; Hereditary Spastic Paraplegia; Heredopathia atactica polyneuritiformis; Herpes zoster oticus; Herpes zoster; Hirayama syndrome; HIV-Associated Dementia and Neuropathy (see also Neurological manifestations of AIDS); Holoprosencephaly; Huntington's disease and other polyglutamine repeat diseases, Hydranencephaly; Hydrocephalus; Hypercortisolism; Hypoxia; Immune-Mediated encephalomyelitis; Inclusion body myositis; Incontinentia pigmenti; Infantile; phytanic acid storage disease; Infantile Refsum disease; Infantile spasms; Inflammatory myopathy; Intracranial cyst; Intracranial hypertension: Joubert syndrome; Kearns-Sayre syndrome; Kennedy disease; Kinsbourne syndrome; Klippel Feil syndrome; Krabbe disease; Kugelberg-Welander disease; Kuru; Lafora disease; Lambert-Eaton myasthenic syndrome; Landau-Kleffner syndrome; Lateral medullary (Wallenberg) syndrome; Learning disabilities; Leigh's disease; Lennox-Gastaut syndrome; Lesch-Nyhan syndrome; Leukodystrophy; Lewy body dementia; Lissencephaly; Locked-In syndrome; Lou Gehrig's disease (aka Motor Neuron Disease or Amyotrophic Lateral Sclerosis); Lumbar disc disease; Lyme disease-Neurological Sequelae; Machado-Joseph disease; Macrencephaly; Megalocephaly; Melkersson-Rosenthal syndrome; Menieres disease; Meningitis; Menkes disease; Metachromatic leukodystrophy; Microcephaly; Migraine; Miller Fisher syndrome; Mini-Strokes; Mitochondrial Myopathies; Mobius syndrome; Monomelic amyotrophy; Motor Neurone Disease; Moyamoya disease; Mucopolysaccharidoses; Multi-Infarct Dementia; Multifocal motor neuropathy; Multiple sclerosis and other demyelinating disorders; Multiple system atrophy with postural hypotension; Muscular dystrophy; Myasthenia gravis; Myelinoclastic diffuse sclerosis; Myoclonic encephalopathy of infants; Myoclonus; Myopathy; Myotonia congenital; Narcolepsy; Neurofibromatosis; Neuroleptic malignant syndrome; Neurological manifestations of AIDS; Neurological sequelae of lupus; Neuromyotonia; Neuronal ceroid lipofuscinosis; Neuronal migration disorders; Niemann-Pick disease; O'Sullivan-McLeod syndrome; Occipital Neuralgia; Occult Spinal Dysraphism Sequence; Ohtahara syndrome; Olivopontocerebellar Atrophy; Opsoclonus Myoclonus; Optic neuritis; Orthostatic Hypotension; Overuse syndrome; Paresthesia; Parkinson's disease; Paramyotonia Congenita; Paraneoplastic diseases; Paroxysmal attacks; Parry Romberg syndrome; Pelizaeus-Merzbacher disease; Periodic Paralyses; Peripheral Neuropathy; Painful Neuropathy and Neuropathic Pain; Persistent Vegetative State; Pervasive developmental disorders; Photic sneeze reflex; Phytanic Acid Storage disease; Pick's disease; Pinched Nerve; Pituitary Tumors; Polymyositis; Porencephaly; Post-Polio syndrome; Postherpetic Neuralgia (PHN); Postinfectious Encephalomyelitis; Postural Hypotension; Prader-Willi syndrome; Primary Lateral Sclerosis; Prion diseases; Progressive; Hemifacial Atrophy; Progressive multifocal leukoencephalopathy; Progressive Sclerosing Poliodystrophy; Progressive Supranuclear Palsy; Pseudotumor cerebri; Ramsay-Hunt syndrome (Type I and Type II); Rasmussen's Encephalitis; Reflex Sympathetic Dystrophy syndrome; Refsum disease, Repetitive Motion Disorders; Repetitive Stress Injuries; Restless Legs syndrome; Retrovirus-Associated Myelopathy; Rett syndrome; Reye's syndrome; Saint Vitus Dance; Sandhoff disease; Schilder's disease; Schizencephaly; Septo-Optic Dysplasia; Shaken Baby syndrome; Shingles; Shy-Drager syndrome; Sjogren's syndrome; Sleep Apnea; Soto's syndrome; Spasticity; Spina bifida; Spinal cord injury; Spinal cord tumors; Spinal Muscular Atrophy; Stiff-Person syndrome; Stroke; Sturge-Weber syndrome; Subacute Sclerosing Panencephalitis; Subarachnoid Hemorrhage; Subcortical Arteriosclerotic Encephalopathy; Sydenham Chorea; Syncope; Syringomyelia; Tardive dyskinesia; Tay-Sachs disease; Temporal arteritis; Tethered Spinal Cord syndrome; Thomsen disease; Thoracic Outlet syndrome; Tic Douloureux; Todd's Paralysis; Tourette syndrome; Transient ischemic attack; Transmissible Spongiform Encephalopathies; Transverse myelitis; Traumatic Brain injury; Tremor; Trigeminal Neuralgia, Tropical Spastic Paraparesis; Tuberous Sclerosis; Vascular Dementia (Multi-Infarct Dementia); Vasculitis including Temporal Arteritis; Von Hippel-Lindau Disease (VHL); Wallenberg's syndrome; Werdnig-Hoffman disease; West syndrome; Whiplash; Williams syndrome; Wilson's disease; and Zellweger syndrome.

A “musculoskeletal disease” or musculoskeletal disorder”, which are used interchangeably, includes a condition that causes or results in muscle atrophy. Muscle atrophy can result from treatment with a glucocorticoid such as cortisol, dexamethasone, betamethasone, prednisone, methylprednisolone or prednisolone. Muscle atrophy can also be a result of denervation due to nerve trauma or a result of degenerative, metabolic or inflammatory neuropathy. For example, muscle atrophy can be a result of an adult motor neuron disease, Guillian-Barré syndrome, infantile spinal muscular atrophy, amyotrophic lateral sclerosis, juvenile spinal muscular atrophy, autoimmune motor neuropathy with multifocal conductor block, paralysis due to stroke or spinal cord injury, skeletal immobilization due to trauma, prolonged bed rest, voluntary inactivity, involuntary inactivity, and metabolic stress or nutritional insufficiency. Muscle atrophy can be a result of myopathy, including for example myotonia; a congenital myopathy, including nemalene myopathy, multi/minicore myopathy and myotubular (centronuclear) myopathy; mitochondrial myopathy; familial periodic paralysis; inflammatory myopathy; metabolic myopathy, such as caused by a glycogen or lipid storage disease; dermatomyositis; polymyositis; inclusion body myositis; myositis ossificans; rhabdomyolysis and myoglobinurias. Myopathy may be caused by a muscular dystrophy syndrome, such as Duchenne muscular dystrophy (DMD), Becker muscular dystrophy (also known as benign pseudohypertrophic muscular dystrophy), myotonic dystrophy, scapulohumeral and facioscapulohumeral muscular dystrophy, Emery-Dreifuss muscular dystrophy, oculopharyngeal muscular dystrophy, limb girdle muscular dystrophy, Fukuyama congenital muscular dystrophy, or hereditary distal myopathy.

Further examples musculoskeletal disease or disorder or conditions that result in musculoskeletal disease or disorder include sarcopenia, skin atrophy, muscle wasting, brain atrophy, atherosclerosis, arteriosclerosis, pulmonary emphysema, osteoporosis, osteoarthritis, immunologic incompetence, high blood pressure, dementia, Huntington's disease, Alzheimer's disease, cataracts, age-related macular degeneration, cancer, stroke, frailty, memory loss, impaired kidney function, metabolic disorders (including Type-II diabetes, metabolic syndrome, hyperglycemia, obesity, thyroid gland disorder), cachexia (including cachexia associated with a rheumatoid arthritis and cachexia associated with cancer), acute and/or chronic renal disease or failure, liver diseases (examples such as fibrosis, cirrhosis), cancer (including rhabdomyosarcoma, prostate cancer, breast cancer, hepatocellular carcinoma, and gastrointestinal cancer), Parkinson's Disease; anemia, exposure to environmental toxins or drugs, HIV/AIDS, fasting, benign congenital hypotonia, central core disease, burn injury, chronic obstructive pulmonary disease, sepsis, congestive heart failure, aging or an age-related condition, and space travel or time spent in a zero gravity environment.

A “painful condition” includes, but is not limited to, neuropathic pain (e.g., peripheral neuropathic pain), central pain, differentiation pain, chronic pain (e.g., chronic nociceptive pain, and other forms of chronic pain such as post-operative pain, e.g., pain arising after hip, knee, or other replacement surgery), pre-operative pain, stimulus of nociceptive receptors (nociceptive pain), acute pain (e.g., phantom and transient acute pain), noninflammatory pain, inflammatory pain, pain associated with cancer, wound pain, burn pain, postoperative pain, pain associated with medical procedures, pain resulting from pruritus, painful bladder syndrome, pain associated with premenstrual dysphoric disorder and/or premenstrual syndrome, pain associated with chronic fatigue syndrome, pain associated with pre-term labor, pain associated with withdrawal symptoms from drug addiction, joint pain, arthritic pain (e.g., pain associated with crystalline arthritis, osteoarthritis, psoriatic arthritis, gouty arthritis, reactive arthritis, rheumatoid arthritis or Reiter's arthritis), lumbosacral pain, musculo-skeletal pain, headache, migraine, muscle ache, lower back pain, neck pain, toothache, dental/maxillofacial pain, visceral pain and the like. One or more of the painful conditions contemplated herein can comprise mixtures of various types of pain provided above and herein (e.g. nociceptive pain, inflammatory pain, neuropathic pain, etc.). In some embodiments, a particular pain can dominate. In other embodiments, the painful condition comprises two or more types of pains without one dominating. A skilled clinician can determine the dosage to achieve a therapeutically effective amount for a particular subject based on the painful condition.

The term “psychiatric disorder” refers to a disease of the mind and includes diseases and disorders listed in the Diagnostic and Statistical Manual of Mental Disorders—Fourth Edition (DSM-IV), published by the American Psychiatric Association, Washington D. C. (1994). Psychiatric disorders include, but are not limited to, anxiety disorders (e.g., acute stress disorder agoraphobia, generalized anxiety disorder, obsessive-compulsive disorder, panic disorder, posttraumatic stress disorder, separation anxiety disorder, social phobia, and specific phobia), childhood disorders, (e.g., attention-deficit/hyperactivity disorder, conduct disorder, and oppositional defiant disorder), eating disorders (e.g., anorexia nervosa and bulimia nervosa), mood disorders (e.g., depression, bipolar disorder, cyclothymic disorder, dysthymic disorder, and major depressive disorder), personality disorders (e.g., antisocial personality disorder, avoidant personality disorder, borderline personality disorder, dependent personality disorder, histrionic personality disorder, narcissistic personality disorder, obsessive-compulsive personality disorder, paranoid personality disorder, schizoid personality disorder, and schizotypal personality disorder), psychotic disorders (e.g., brief psychotic disorder, delusional disorder, schizoaffective disorder, schizophreniform disorder, schizophrenia, and shared psychotic disorder), substance-related disorders (e.g., alcohol dependence, amphetamine dependence, cannabis dependence, cocaine dependence, hallucinogen dependence, inhalant dependence, nicotine dependence, opioid dependence, phencyclidine dependence, and sedative dependence), adjustment disorder, autism, delirium, dementia, multi-infarct dementia, learning and memory disorders (e.g., amnesia and age-related memory loss), and Tourette's disorder.

The term “metabolic disorder” refers to any disorder that involves an alteration in the normal metabolism of carbohydrates, lipids, proteins, nucleic acids, or a combination thereof. A metabolic disorder is associated with either a deficiency or excess in a metabolic pathway resulting in an imbalance in metabolism of nucleic acids, proteins, lipids, and/or carbohydrates. Factors affecting metabolism include, and are not limited to, the endocrine (hormonal) control system (e.g., the insulin pathway, the enteroendocrine hormones including GLP-1, PYY or the like), the neural control system (e.g., GLP-1 in the brain), or the like. Examples of metabolic disorders include, but are not limited to, diabetes (e.g., type 1 diabetes, type 2 diabetes, gestational diabetes), hyperglycemia, hyperinsulinemia, insulin resistance, and obesity.

In some aspects, the therapeutic agent is one or more of monoclonal antibodies, chimeric antibodies, humanized antibodies, nanobodies, antibody fragments, cholesterol, hormones, peptides, proteins, chemotherapeutics, antineoplastic agents, low molecular weight drugs, vitamins, co-factors, nucleosides, nucleotides, oligonucleotides, polynucleotides, enzymatic nucleic acids, antisense nucleic acids, triplex forming oligonucleotides, antisense DNA or RNA compositions, chimeric DNA:RNA compositions, allozymes, aptamers, ribozyme, decoys, analogs, plasmids, expression vectors, small nucleic acid molecules, mRNA, RNAi agents, short interfering nucleic acid (siNA), short interfering RNA (siRNA), double-stranded RNA (dsRNA), micro-RNA (miRNA), short hairpin RNA (shRNA), peptide nucleic acid (PNA), locked nucleic acid ribonucleotides (LNA), morpholino nucleotides, threose nucleic acid (TNA), glycol nucleic acid (GNA), sisiRNA (small internally segmented interfering RNA), aiRNA (asymmetrical interfering RNA), and siRNA with 1, 2, or more mismatches between the sense and anti-sense strand to relevant cells or tissues.

For example, the therapeutic agent may comprise a nucleic acid, such as mRNA, for treatment or preventing of a disease state. In particular, the disease may include one of the diseases mentioned above (e.g. neurological diseases, hepatic disease, musculoskeletal disease). Lipid particles of the present disclosure may afford greater permeation through the blood-brain barrier, thereby increasing effectivity of delivery of certain therapeutic agents.

In vivo application of the disclosed compounds, and compositions containing them, can be accomplished by any suitable method and technique presently or prospectively known to those skilled in the art. For example, the disclosed compounds can be formulated in a physiologically- or pharmaceutically-acceptable form and administered by any suitable route known in the art including, for example, oral, nasal, rectal, topical, and parenteral routes of administration. As used herein, the term parenteral includes subcutaneous, intradermal, intravenous, intramuscular, intraperitoneal, and intrasternal administration, such as by injection. In certain aspects, the administration can include intravenous, intrathecal, intracranial, also intramuscular, intratumoral, intratracheal, subcutaneous application. Administration of the disclosed compounds or compositions can be a single administration, or at continuous or distinct intervals as can be readily determined by a person skilled in the art.

The compounds disclosed herein, and compositions comprising them, can also be administered utilizing liposome technology, slow release capsules, implantable pumps, and biodegradable containers. These delivery methods can, advantageously, provide a uniform dosage over an extended period of time. The compounds can also be administered in their salt derivative forms or crystalline forms.

The compounds disclosed herein can be formulated according to known methods for preparing pharmaceutically acceptable compositions. Formulations are described in detail in a number of sources which are well known and readily available to those skilled in the art. For example, Remington's Pharmaceutical Science by E. W. Martin (1995) describes formulations that can be used in connection with the disclosed methods. In general, the compounds disclosed herein can be formulated such that an effective amount of the compound is combined with a suitable excipient in order to facilitate effective administration of the compound. The compositions used can also be in a variety of forms. These include, for example, solid, semi-solid, and liquid dosage forms, such as tablets, pills, powders, liquid solutions or suspension, suppositories, injectable and infusible solutions, and sprays. The preferred form depends on the intended mode of administration and application. The compositions can also include conventional pharmaceutically-acceptable carriers and diluents which are known to those skilled in the art.

Examples of carriers or diluents for use with the compounds include ethanol, dimethyl sulfoxide, glycerol, alumina, starch, saline, and equivalent carriers and diluents. To provide for the administration of such dosages for the desired application, compositions disclosed herein can comprise between about 0.1% and 100% by weight of the total of one or more of the subject compounds based on the weight of the total composition including carrier or diluent.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

Formulations suitable for administration include, for example, aqueous sterile injection solutions, which can contain antioxidants, buffers, bacteriostats, and solutes that render the formulation isotonic with the blood of the intended recipient; and aqueous and nonaqueous sterile suspensions, which can include suspending agents and thickening agents. The formulations can be presented in unit-dose or multi-dose containers, for example sealed ampoules and vials, and can be stored in a freeze dried (lyophilized) condition requiring only the condition of the sterile liquid carrier, for example, water for injections, prior to use. Extemporaneous injection solutions and suspensions can be prepared from sterile powder, granules, tablets, etc. It should be understood that in addition to the excipients particularly mentioned above, the compositions disclosed herein can include other agents conventional in the art having regard to the type of formulation in question.

Compounds disclosed herein, and compositions comprising them, can be delivered to a cell either through direct contact with the cell or via a carrier means. Carrier means for delivering compounds and compositions to cells are known in the art.

For the treatment of oncological disorders, the compounds or compositions disclosed herein can be administered to a patient in need of treatment in combination with other antitumor or anticancer substances and/or with radiation and/or photodynamic therapy and/or with surgical treatment to remove a tumor. These other substances or treatments can be given at the same as or at different times from the compounds or compositions disclosed herein. For example, the compounds or compositions disclosed herein can be used in combination with mitotic inhibitors such as taxol or vinblastine, alkylating agents such as cyclophosamide or ifosfamide, antimetabolites such as 5-fluorouracil or hydroxyurea, DNA intercalators such as adriamycin or bleomycin, topoisomerase inhibitors such as etoposide or camptothecin, antiangiogenic agents such as angiostatin, antiestrogens such as tamoxifen, and/or other anticancer drugs or antibodies, such as, for example, GLEEVEC (Novartis Pharmaceuticals Corporation) and HERCEPTIN (Genentech, Inc.), respectively, or an immunotherapeutic such as ipilimumab and bortezomib.

In certain examples, compounds and compositions disclosed herein can be locally administered at one or more anatomical sites, such as sites of unwanted cell growth (such as a tumor site or benign skin growth, e.g., injected or topically applied to the tumor or skin growth), optionally in combination with a pharmaceutically acceptable carrier such as an inert diluent. Compounds and compositions disclosed herein can be systemically administered, such as intravenously or orally, optionally in combination with a pharmaceutically acceptable carrier such as an inert diluent, or an assimilable edible carrier for oral delivery. They can be enclosed in hard or soft shell gelatin capsules, can be compressed into tablets, or can be incorporated directly with the food of the patient's diet. For oral therapeutic administration, the active compound can be combined with one or more excipients and used in the form of ingestible tablets, buccal tablets, troches, capsules, elixirs, suspensions, syrups, wafers, aerosol sprays, and the like.

The tablets, troches, pills, capsules, and the like can also contain the following: binders such as gum tragacanth, acacia, corn starch or gelatin; diluents such as dicalcium phosphate; a disintegrating agent such as corn starch, potato starch, alginic acid and the like: a lubricant such as magnesium stearate; and a sweetening agent such as sucrose, fructose, lactose or aspartame or a flavoring agent such as peppermint, oil of wintergreen, or cherry flavoring can be added. When the unit dosage form is a capsule, it can contain, in addition to materials of the above type, a liquid carrier, such as a vegetable oil or a polyethylene glycol. Various other materials can be present as coatings or to otherwise modify the physical form of the solid unit dosage form. For instance, tablets, pills, or capsules can be coated with gelatin, wax, shellac, or sugar and the like. A syrup or elixir can contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound can be incorporated into sustained-release preparations and devices.

Compounds and compositions disclosed herein, including pharmaceutically acceptable salts thereof, can be administered intravenously, intramuscularly, or intraperitoneally by infusion or injection. Solutions of the active agent or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the active ingredient, which are adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Optionally, the prevention of the action of microorganisms can be brought about by various other antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the inclusion of agents that delay absorption, for example, aluminum monostearate and gelatin.

Pharmaceutical compositions disclosed herein suitable for injectable use include sterile aqueous solutions or dispersions. Furthermore, the compositions can be in the form of sterile powders for the extemporaneous preparation of such sterile injectable solutions or dispersions. In some examples, the final injectable form can be sterile and can be effectively fluid for easy syringability. In some examples, the pharmaceutical compositions can be stable under the conditions of manufacture and storage; thus, they can be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof

Sterile injectable solutions are prepared by incorporating a compound and/or agent disclosed herein in the required amount in the appropriate solvent with various other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

Pharmaceutical compositions disclosed herein can be in a form suitable for topical use such as, for example, an aerosol, cream, ointment, lotion, dusting powder, mouth washes, gargles, solution, tincture, and the like. In some examples, the compositions can be in a form suitable for use in transdermal devices. In some examples, it will be desirable to administer them topically to the skin as compositions, in combination with a dermatologically acceptable carrier, which can be a solid or a liquid. Compounds and agents and compositions disclosed herein can be applied topically to a subject's skin. These formulations can be prepared, utilizing any of the compounds disclosed herein or pharmaceutically acceptable salts thereof, via conventional processing methods.

Useful solid carriers include finely divided solids such as talc, clay, microcrystalline cellulose, silica, alumina and the like. Useful liquid carriers include water, alcohols or glycols or water-alcohol/glycol blends, in which the compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers, for example.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Pharmaceutical compositions disclosed herein can be in a form suitable for rectal administration wherein the carrier is a solid. In some examples, the mixture forms unit dose suppositories. Suitable carriers include cocoa butter and other materials commonly used in the art. The suppositories can be conveniently formed by first admixing the composition with the softened or melted carriers) followed by chilling and shaping in molds.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing any of the compounds disclosed herein, and/or pharmaceutically acceptable salts thereof, can also be prepared in powder or liquid concentrate form.

### Methods of Making

Methods of making the lipid nanomaterials disclosed herein are provided herein. The lipid nanomaterials preferably has a particle size in the range of 10 to 1000 nm and exhibit enhanced targetability, for example, which is achieved as a result of the morphology and properties of the lipid nanoparticles. The lipid nanoparticles can be prepared in accordance with the present disclosure.

While the invention has been described with reference to various and preferred embodiments, it should be understood by those skilled in the art that various changes may be made and equivalents may be substituted for elements thereof without departing from the essential scope of the invention. In addition, many modifications may be made to adapt a particular situation or material to the teachings of the invention without departing from the essential scope thereof.

Therefore, it is intended that the invention not be limited to the particular embodiment(s) disclosed herein contemplated for carrying out this invention, but that the invention will include all embodiments falling within the scope of the claims.

Publications cited herein are hereby specifically incorporated by reference in their entireties and at least for the material for which they are cited.

### EXAMPLES

### Example 1: Brain Targeting Lipid Nanomaterials and Uses Thereof

The efficient delivery of mRNA is a key step and challenge for mRNA therapeutics. Despite promising data from ongoing clinical trials, the clinical use of mRNA requires the discovery and development of more efficient delivery systems.

### SUMMARY

Described in this Example are brain targeting lipid nanomaterials for gene therapy and drug delivery applications.

### General Procedure for the Synthesis of Brain Targeting Lipids:

(1) To a solution of (Fmoc protected) brain targeting ligands, DMF and N,N-diisopropylethylamine. N,N,N′,N′-Tetramethyl-O-(3,4-dihydro-4-oxo-1,2,3-benzotriazin-3-yl)uranium tetrafluoroborate) was added. The resulting mixture was stirred for 20 min at room temperature. Then Boc-protected amines was added, and the solution was stirred at room temperature overnight. The product mixture was added, dropwise, into 200 mL of 5% NaHCO₃ and the extracted by ethyl acetate and washed 3 times with deionized water. The resulting solid was further purified by Combiflash column chromatography with a RediSep Gold Resolution silica column with gradient elution from 100% CH₂Cl₂ to CH₂Cl₂/MeOH (85/15, v/v/v) to give Boc-protected brain targeting amines.

(2) The Boc-protected brain targeting amine was dissolved in 10 mL of CH₂Cl₂ and 1 mL of CF₃COOH and the mixture stirred for 2.5 h. After the reaction was completed checked by TLC, the solvent was removed under reduced pressure to afford the brain targeting amine.

(3) To a solution of the amine (0.2 mmol) and triethylamine (1.0 mmol) in THF (5 mL) was added aldehyde (1.0 mmol) and sodium triacetoxyborohydride (1.2 mmol). Then the resulting mixture was stirred at room temperature overnight. The resulting product was washed with NaHCO₃ (50 ml) and brine (50 ml), purified by Combiflash column chromatography with a RediSep Gold Resolution silica column (Teledyne Isco) using gradient elution from 100% CH₂Cl₂ to and CH₂Cl₂/MeOH/NH₄OH (80/20/0.5, v/v/v) to afford the final products. Fmoc was removed by adding piperidine in CH₂Cl₂.

Synthetic strategies for the production of example classes of lipids are detailed in Schemes 1-4 below.

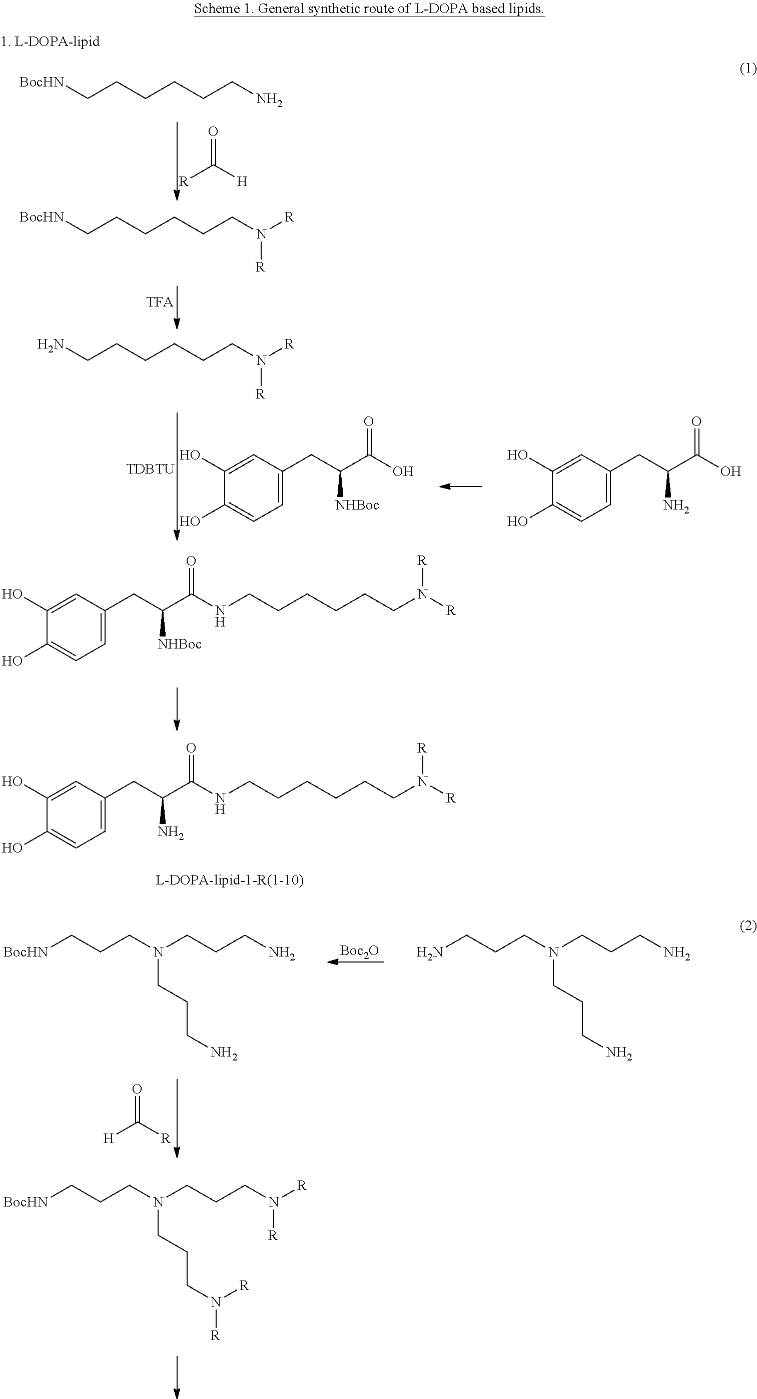

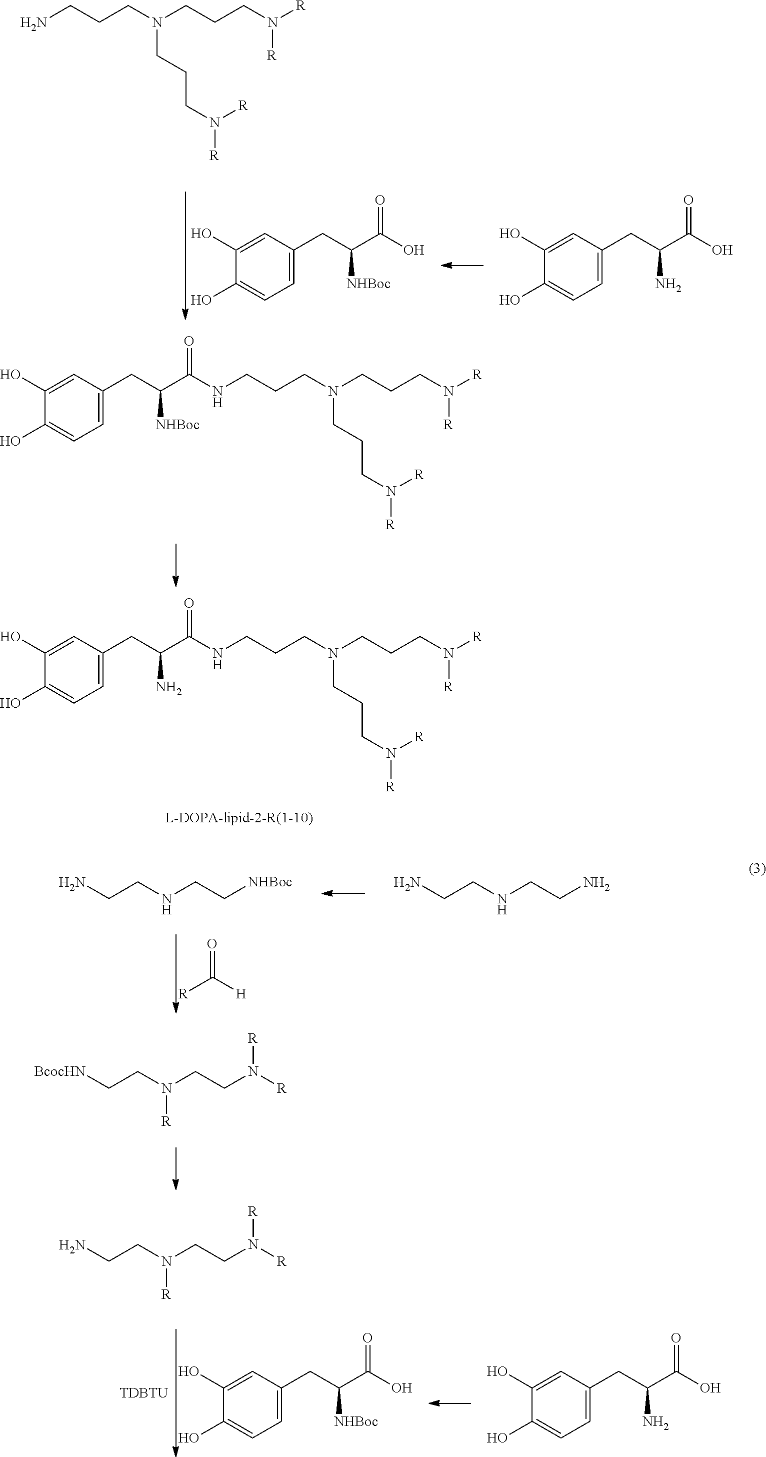

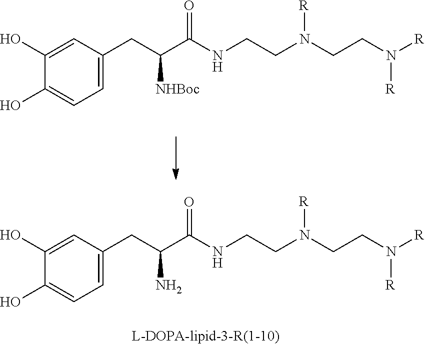

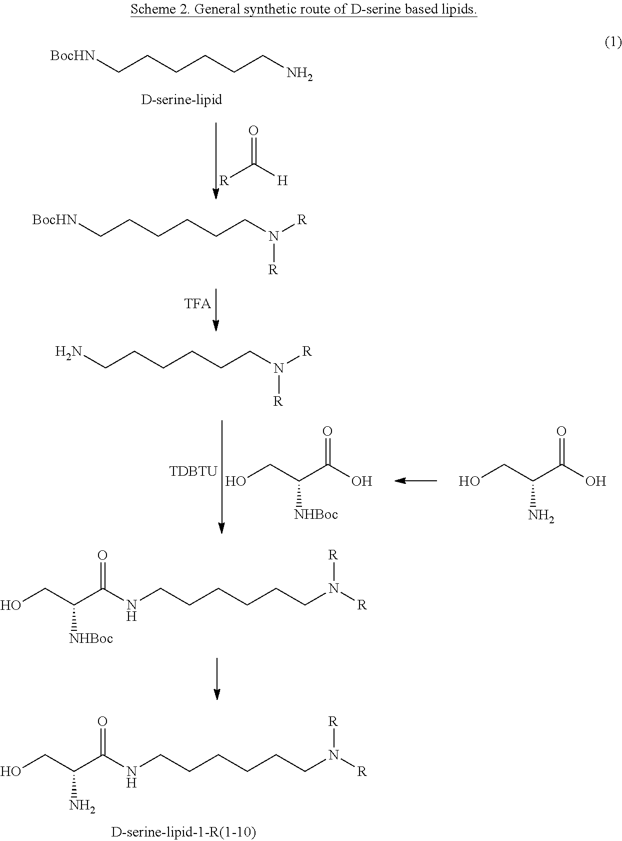

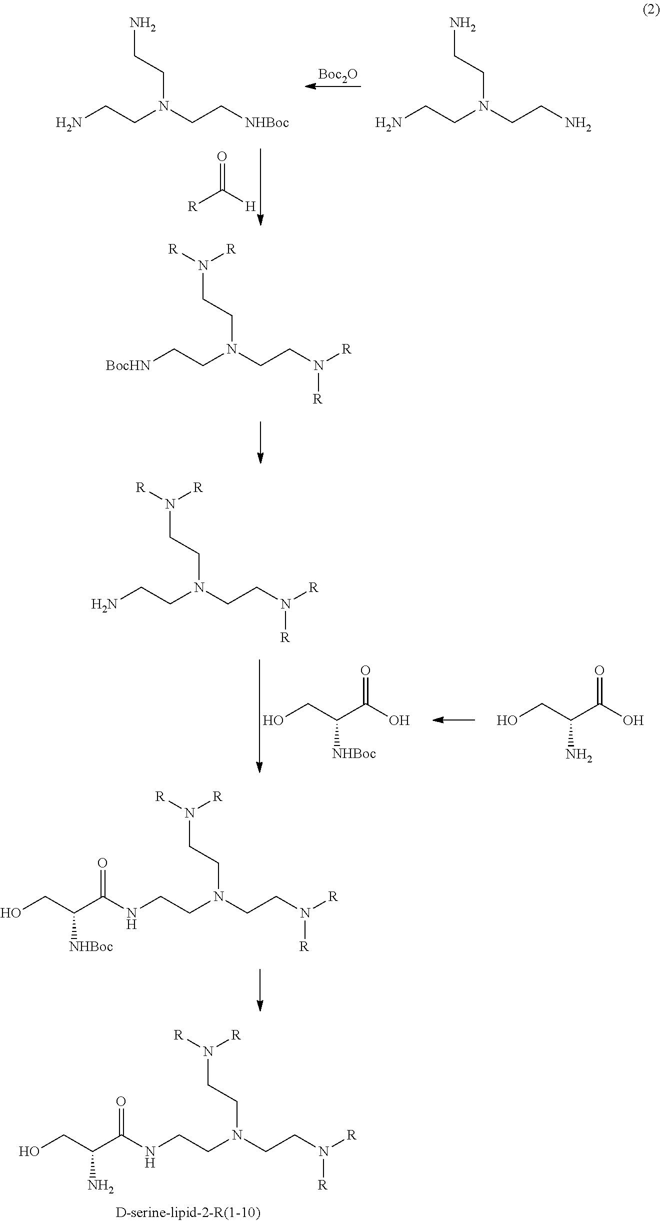

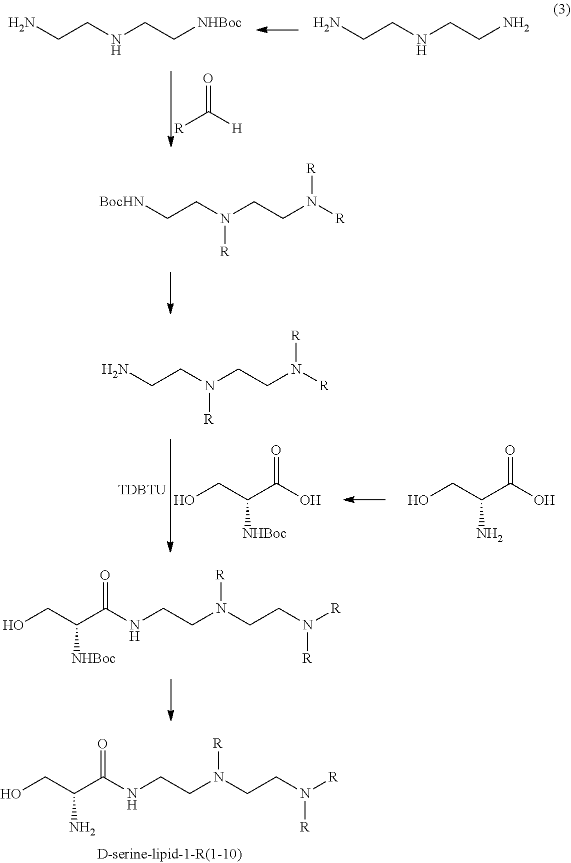

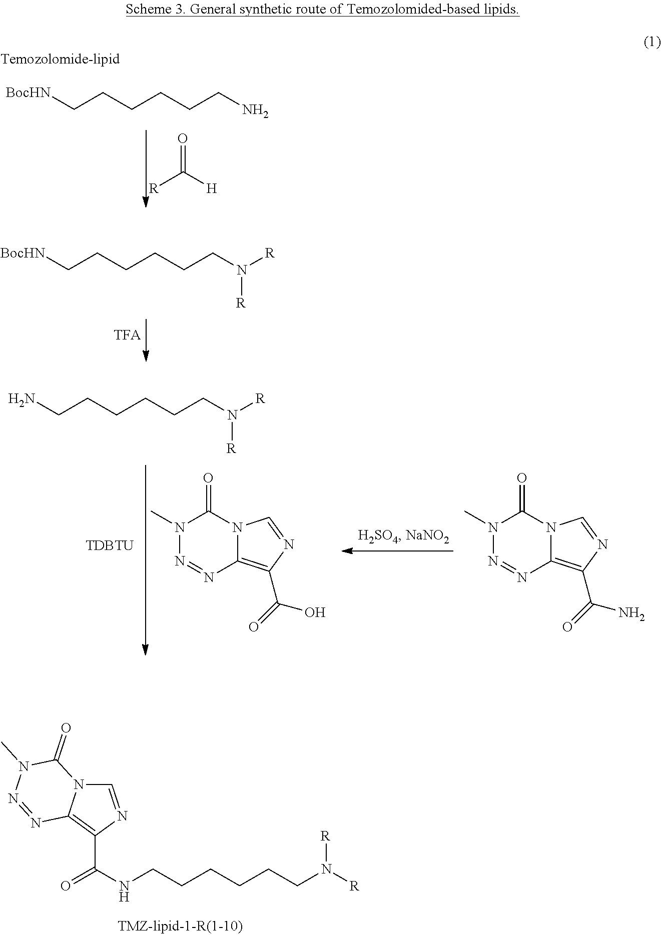

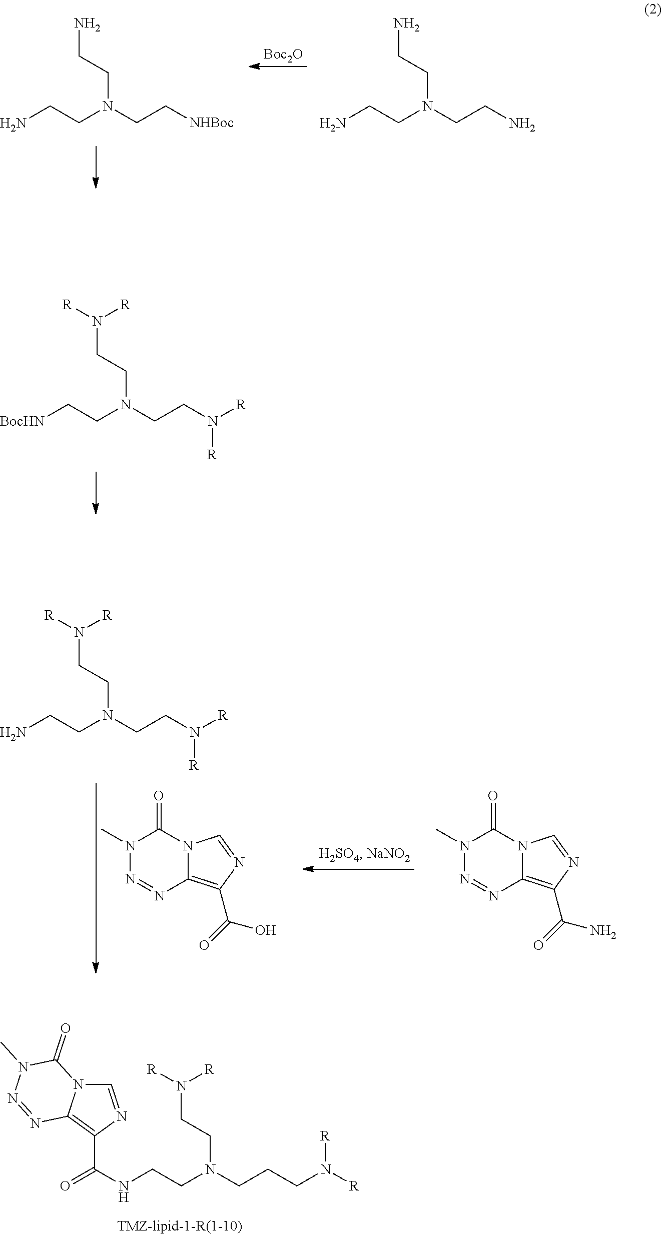

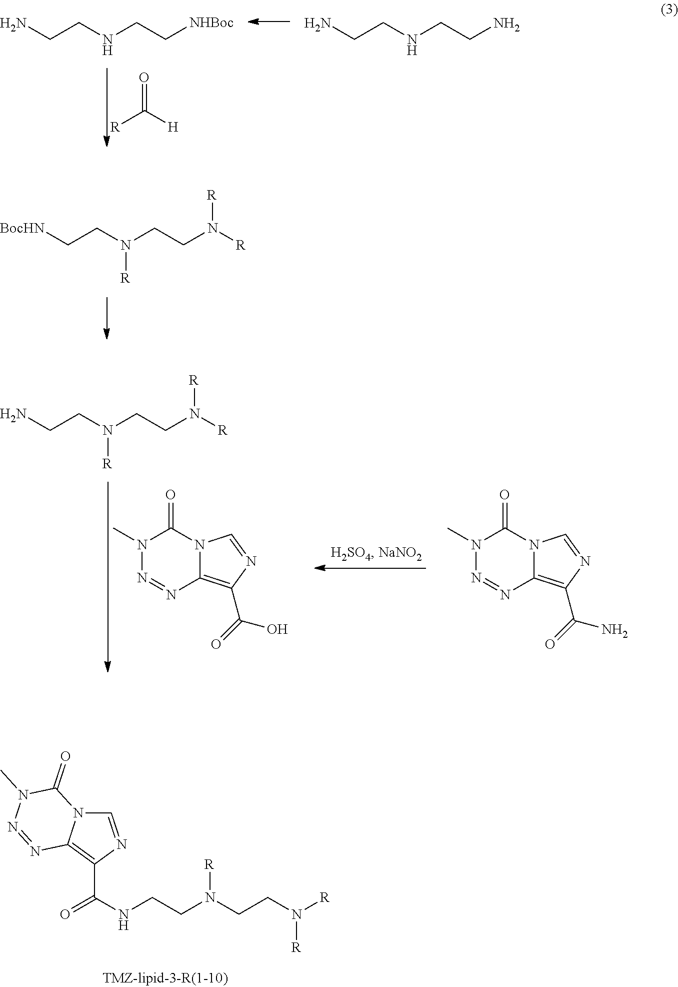

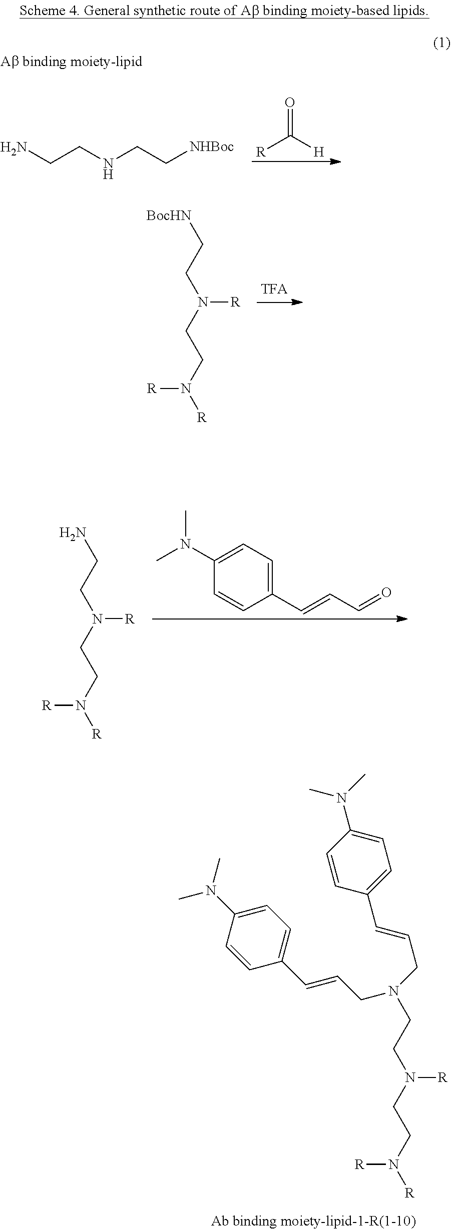

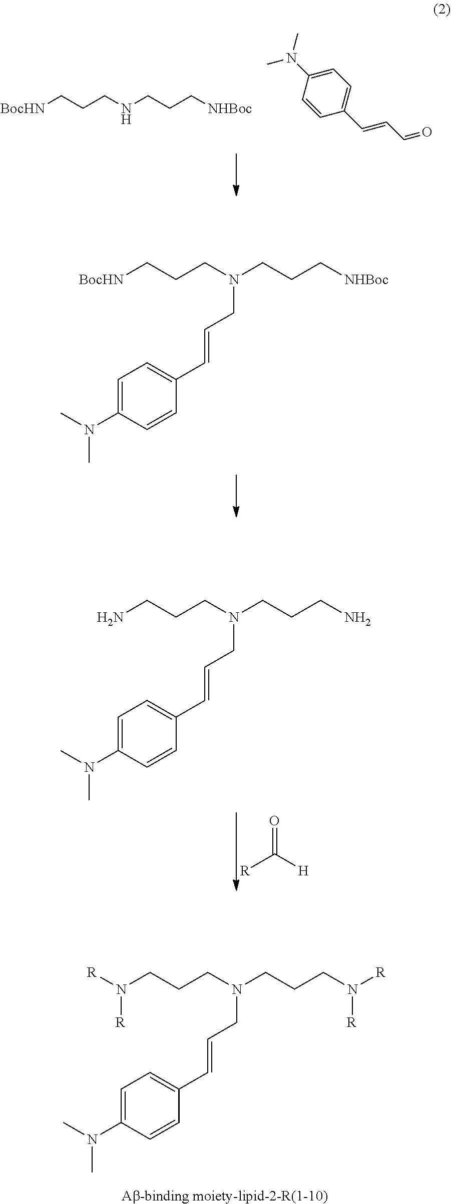

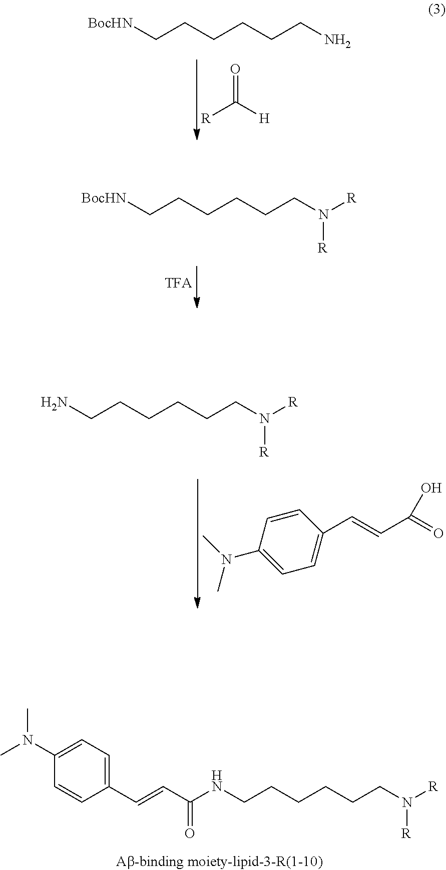

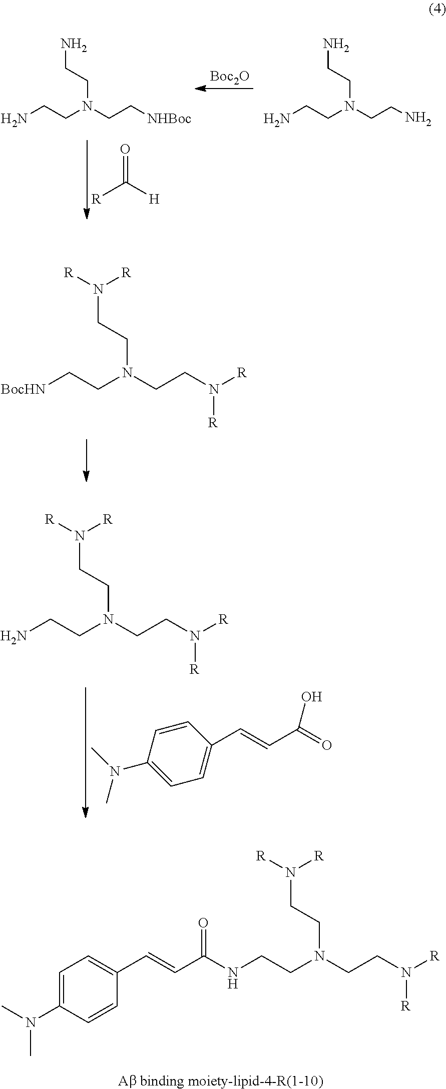

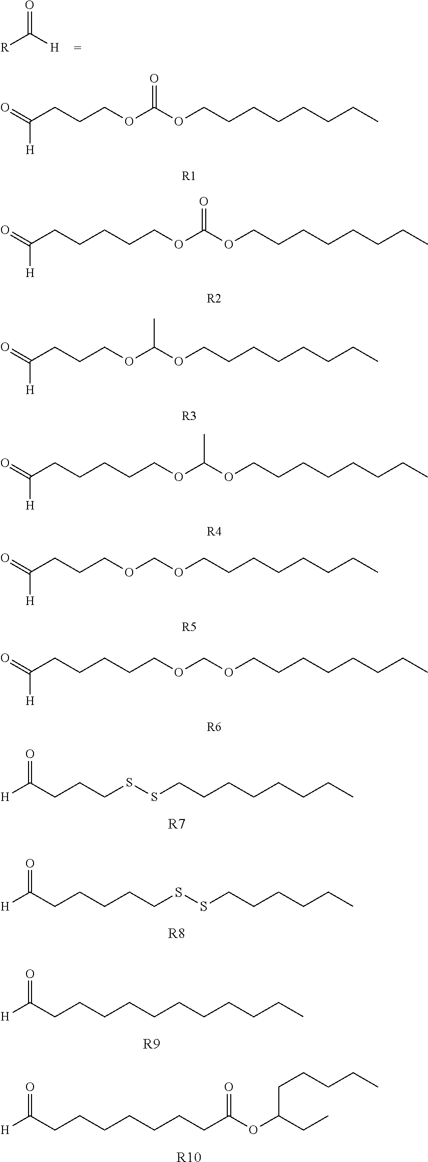

### Lipid Characterization:

The characterization of example lipids is included below.

L-DOPA-lipid-1-R9: ¹H NMR (400 MHz, CDCl₃) δ 6.71 (s, 2H), 6.50 (s, 1H), 4.19 (s, 1H), 3.01 (d, J=38.3 Hz, 11H), 1.63 (s, 6H), 1.47-1.01 (m, 45H), 0.89 (t, J=6.8 Hz, 6H). ESI-MS [M+H⁺] Found 632.8.

L-DOPA-lipid-2-R9: ¹H NMR (400 MHz, CDCl₃) δ 6.93-6.65 (m, 2H), 6.53 (d, J=6.0 Hz, 1H), 4.31-4.03 (m, 1H), 3.45-2.78 (m, 20H), 2.01-1.43 (m, 10H), 1.28 (d, J=11.6 Hz, 71H), 0.90 (t, J=6.8 Hz, 12H). ESI-MS [M+2H⁺] Found 521.2.

D-serine-lipid-1-R9: ¹H NMR (300 MHz, CDCl₃) δ 4.22 (s, 1H), 4.06 (s, 1H), 3.77 (s, 1H), 3.40-3.15 (m, 2H), 3.04 (m, 6H), 1.60 (m, 6H), 1.30 (m, 40H), 0.89 (t, J=6.7 Hz, 6H). MALDI-MS [M+H⁺] Found 540.449.

D-serine-lipid-2-R9: ¹H NMR (300 MHz, CDCl₃) δ 4.20 (s, 1H), 4.00 (s, 1H), 3.77 (s, 1H), 3.40-3.05 (m, 17H), 1.57 (d, J=49.7 Hz, 10H), 1.27 (s, 70H), 0.89 (t, J=6.6 Hz, 12H). MALDI-MS [M+H⁺] Found 907.082.

TMZ-lipid-1-R9: ¹H NMR (300 MHz, CDCl₃) δ 8.39 (s, 1H), 4.04 (s, 3H), 3.71-3.21 (m, 2H), 2.56 (m, 6H), 1.81-1.01 (m, 56H), 0.88 (t, J=6.6 Hz, 6H). MALDI-MS [M+H⁺] Found 630.653.

TMZ-lipid-2-R9: ¹H NMR (400 MHz, CDCl₃) δ 8.49-8.15 (m, 1H), 4.21-3.81 (m, 3H), 3.78-3.54 (m, 2H), 2.76 (m, 10H), 2.50 (m, 8H), 1.59-1.04 (m, 73H), 0.90 (t, J=6.8 Hz, 12H). MALDI-MS [M+H⁺] Found 996.899.

Aβ binding moiety-lipid-1-R9: ¹H NMR (300 MHz, CDCl₃) δ7.26 (m, 4H), 6.74 (m, 4H), 6.49 (dd, 2H), 6.25-5.87 (m, 2H), 3.27 (t, J=19.7 Hz, 4H), 2.94 (d, J=15.3 Hz, 12H), 2.48 (m, 10H), 1.35 (d, J=43.5 Hz, 59H), 0.90 (t, J=6.6 Hz, 9H). MALDI-MS [M+H⁺] Found 926.838.

Aβ binding moiety-lipid-2-R9: ¹H NMR (300 MHz, CDCl₃) δ 7.28 (t, 2H), 6.69 (d, 2H), 6.42 (d, J=15.7 Hz, 1H), 6.17-5.85 (m, 1H), 3.28 (t, 2H), 2.94 (m, 6H), 2.69-2.09 (m, 16H), 1.35 (d, J=44.4 Hz, 82H), 1.05-0.71 (m, 12H). MALDI-MS [M+H⁺] Found 961.923.

Aβ binding moiety-lipid-3-R9: ¹H NMR (400 MHz, CDCl₃) δ 7.29 (m, 4H), 6.67 (m, 4H), 6.52 (dd, 2H), 6.22-5.88 (m, 2H), 3.45 (t, 2H), 2.98 (s, 10H), 2.75 (m, 6H), 1.64 (s, 6H), 1.43-1.09 (m, 36H), 0.90 (t, J=6.8 Hz, 6H). MALDI-MS [M+H⁺] Found 771.680.

### Example 2: Evaluation of Lipids BL1-BL83

### General Procedure for the Synthesis of Brain Targeting Lipids:

(1) To a solution of (Fmoc protected) brain targeting ligands, DMF and N,N-diisopropylethylamine. N,N,N′,N′-Tetramethyl-O-(3,4-dihydro-4-oxo-1,2,3-benzotriazin-3-yl)uranium tetrafluoroborate) was added. The resulting mixture was stirred for 20 min at room temperature. Then Boc-protected amines was added, and the solution was stirred at room temperature overnight. The product mixture was added, dropwise, into 200 mL of 5% NaHCO₃ and the extracted by ethyl acetate and washed 3 times with deionized water. The resulting solid was further purified by Combiflash column chromatography with a RediSep Gold Resolution silica column with gradient elution from 100% CH₂Cl₂ to CH₂Cl₂/MeOH (85/15, v/v/v) to give Boc-protected brain targeting amines.

(2) The Boc-protected brain targeting amine was dissolved in 10 mL of CH₂Cl₂ and 1 mL of CF₃COOH and the mixture stirred for 2.5 h. After the reaction was completed checked by TLC, the solvent was removed under reduced pressure to afford the brain targeting amine.

(3) To a solution of the amine (0.2 mmol) and triethylamine (1.0 mmol) in THF (5 mL) was added aldehyde (1.0 mmol) and sodium triacetoxyborohydride (1.2 mmol). Then the resulting mixture was stirred at room temperature overnight. The resulting product was washed with NaHCO₃ (50 ml) and brine (50 ml), purified by Combiflash column chromatography with a RediSep Gold Resolution silica column (Teledyne Isco) using gradient elution from 100% CH₂Cl₂ to and CH₂Cl₂/MeOH/NH₄OH (80/20/0.5, v/v/v) to afford the final products. Fmoc was removed by adding piperidine in CH₂Cl₂.

The synthetic strategies used to access particular lipids are detailed in the schemes below.

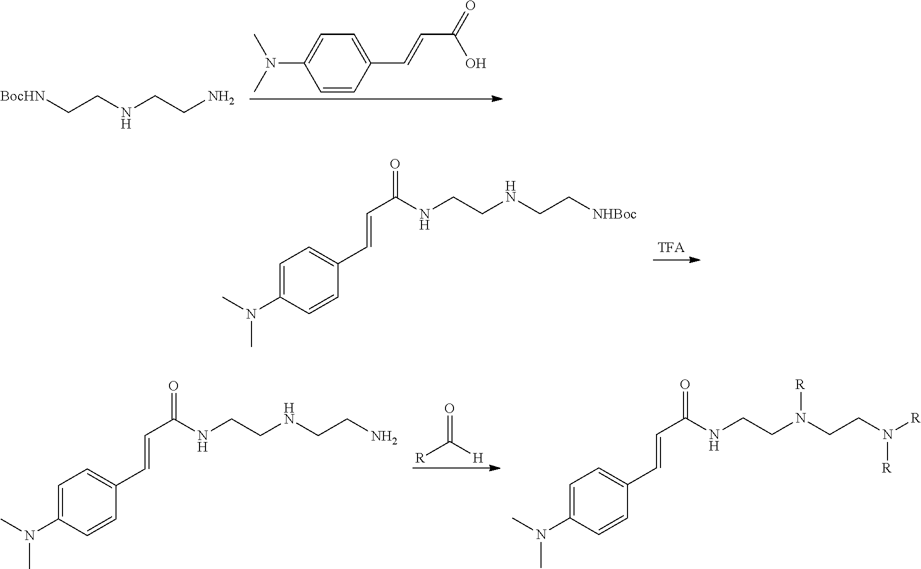

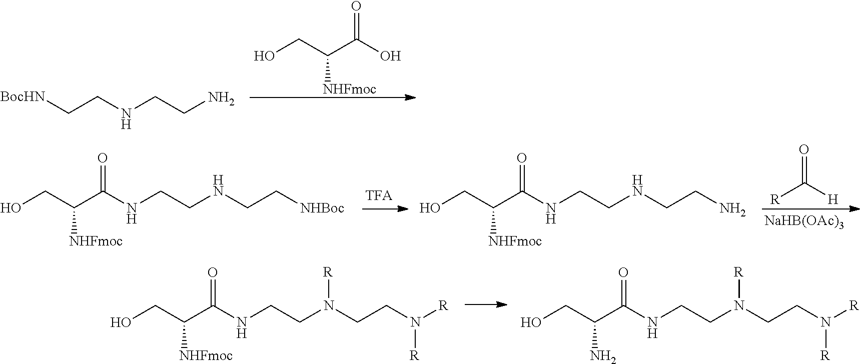

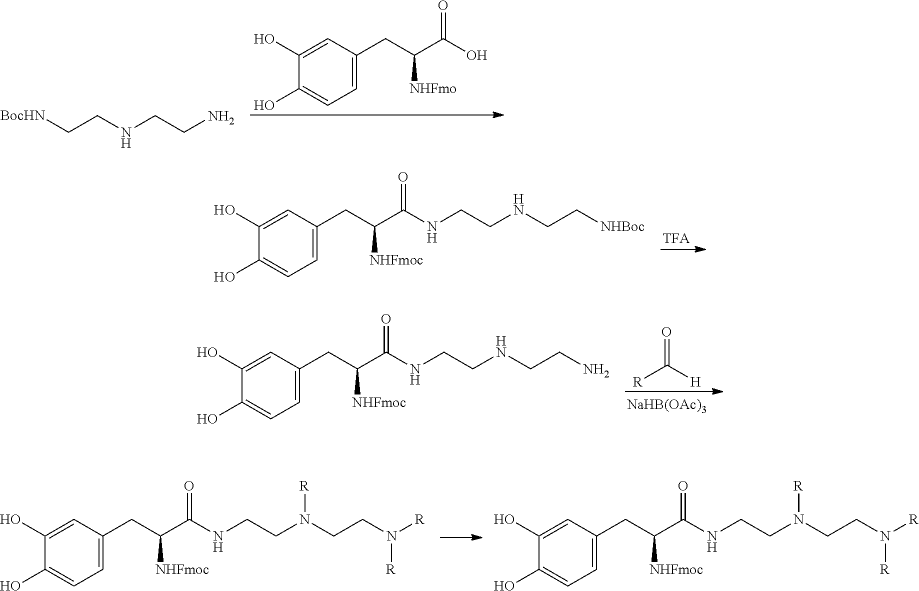

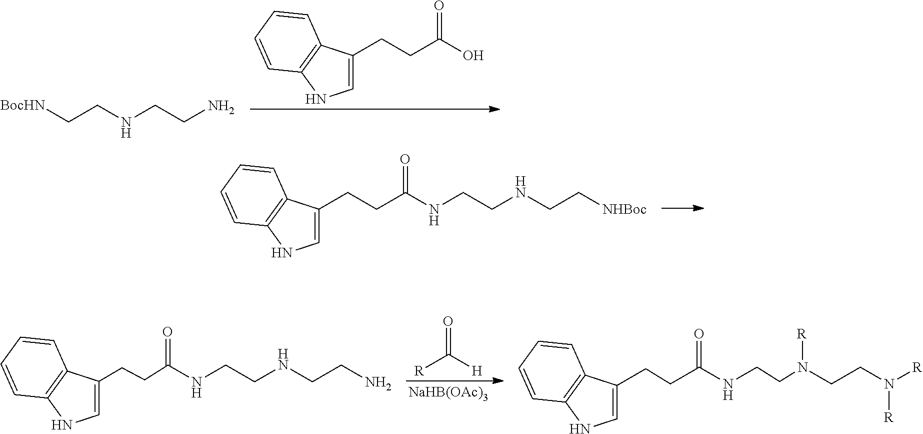

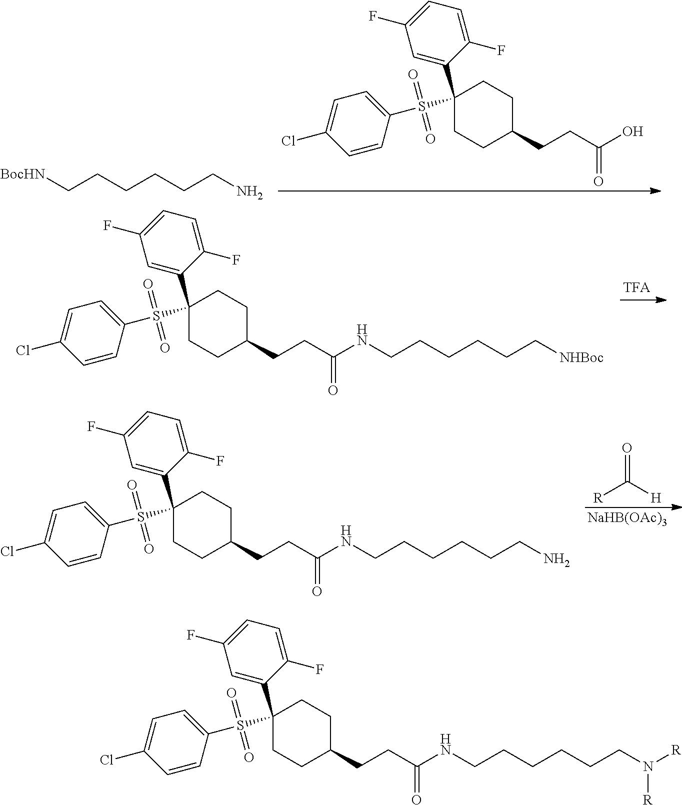

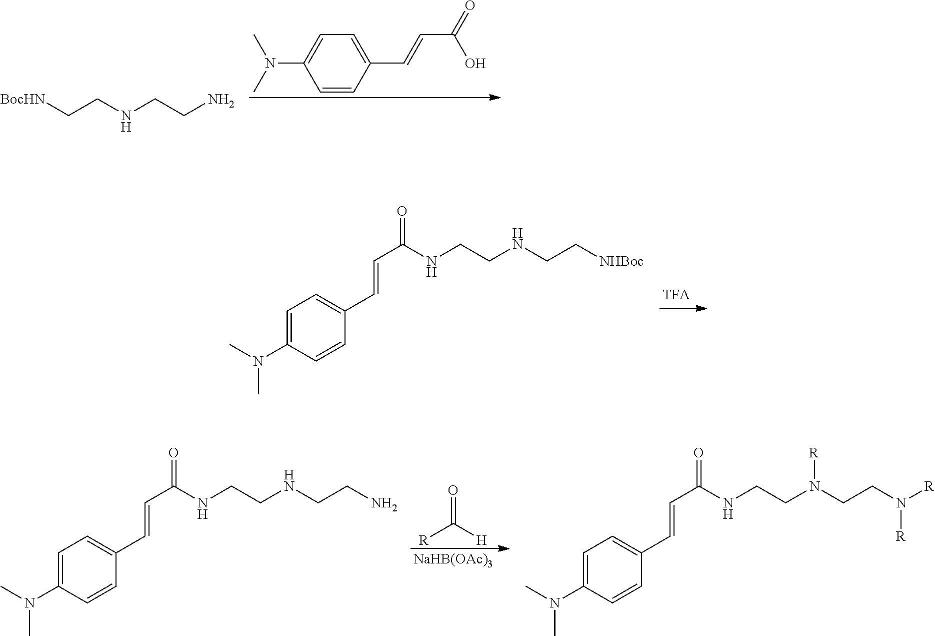

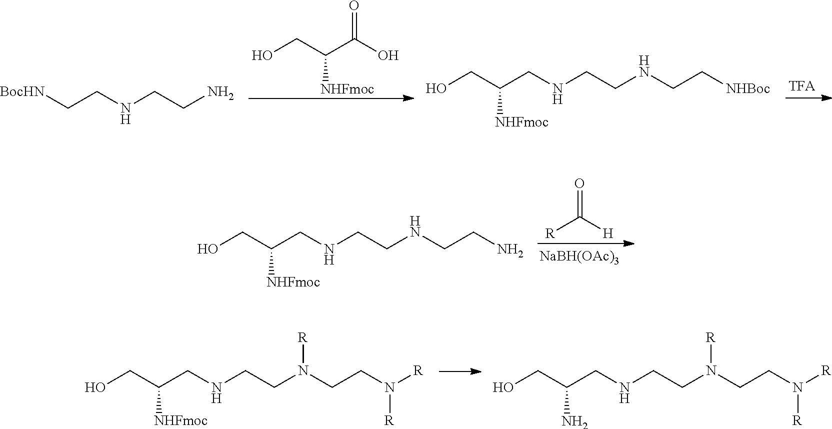

### Lipid Characterizations:

The characterization of example lipids is included below.

BL1: ¹H NMR (400 MHz, CDCl₃) δ 6.71 (m, 2H), 6.50 (d, 1H), 4.19 (m, 1H), 3.01 (m, 10H), 1.63-1.01 (m, 50H), 0.89 (t, J=6.8 Hz, 6H). ESI-MS for C₃₉H₇₄N₃O₃ ([M+H]⁺) Found: 632.8.

BL2: ¹H NMR (400 MHz, CDCl₃) δ 6.93-6.65 (m, 2H), 6.53 (d, J=6.0 Hz, 1H), 4.31-4.03 (m, 1H), 3.45-2.78 (m, 20H), 2.01-1.43 (m, 10H), 1.28 (m, 71H), 0.90 (t, J=6.8 Hz, 12H). ESI-MS for C₆₆H₁₂₉N₅O₃ ([M+2H]²⁺) Found 521.2.

BL3: ¹H NMR (400 MHz, CDCl₃) δ 6.75 (m, 2H), 6.60 (d, 1H), 4.57-4.20 (m, 4H), 4.16 (m, 8H), 2.97 (m, 12H), 1.95-1.06 (m, 33H), 0.91 (t, J=6.8 Hz, 6H).

BL4: ¹H NMR (400 MHz, CDCl₃) δ 6.88-6.35 (m, 2H), 6.21-5.96 (m, 1H), 4.83-4.51 (m, 4H), 4.53-4.20 (m, 4H), 3.31-2.29 (m, 12H), 1.93-1.02 (m, 46H), 0.90 (t, J=6.8 Hz, 6H).

BL5: ¹H NMR (400 MHz, CDCl₃) δ 6.75 (m, 2H), 6.60 (s, 1H), 4.57-4.20 (m, 4H), 4.16 (m, 6H), 2.97 (m, 12H), 1.95-1.06 (m, 33H), 0.91 (t, J=6.8 Hz, 6H).

BL6: ¹H NMR (400 MHz, CDCl₃) δ 6.88-6.48 (m, 3H), 4.47-4.24 (m, 3H), 4.15 (m, 10H), 2.93-2.21 (m, 11H), 1.89-1.10 (m, 43H), 0.81 (t, J=6.8 Hz, 9H). ESI-MS for C₅₂H₉₆N₄O₁₂ ([M+2H]²⁺) Found 484.5.

BL7: ¹H NMR (400 MHz, CDCl₃) δ 6.76 (m, 3H), 4.63 (m, 3H), 4.47-4.14 (m, 4H), 3.77-2.29 (m, 30H), 1.96-0.99 (m, 55H), 0.96-0.74 (t, J=6.8 Hz, 9H). ESI-MS for C₅₅H₁₀₆N₄O₉ ([M+2H]²⁺) Found 484.5.

BL8: ¹H NMR (400 MHz, CDCl₃) δ 6.99-6.45 (m, 4H), 4.64 (m, 4H), 4.44-3.86 (m, 7H), 3.74-2.25 (m, 33H), 1.85-1.04 (m, 78H), 0.89 (t, J=6.8 Hz, 9H).

BL9: ¹H NMR (400 MHz, CDCl₃) δ 7.02-6.44 (m, 4H), 4.81-4.54 (m, 7H), 4.45-4.13 (m, 4H), 3.67-3.37 (m, 14H), 3.18-2.21 (m, 16H), 1.95-1.06 (m, 49H), 0.91 (t, J=6.5 Hz, 9H).

BL10: ¹H NMR (400 MHz, CDCl₃) δ 6.76 (t, 1H), 6.72-6.59 (m, 1H), 6.56-6.42 (m, 1H), 2.91-2.77 (m, 2H), 2.68 (m, 6H), 1.78-1.06 (m, 46H), 0.90 (t, J=6.8 Hz, 6H).

BL11: ¹H NMR (300 MHz, CDCl₃) δ 6.75 (t, 1H), 6.63 (d, 1H), 6.50 (m, 1H), 4.13 (m, 8H), 2.94-2.28 (m, 8H), 1.96-1.03 (m, 51H), 0.89 (t, J=6.7 Hz, 6H).

BL12: ¹H NMR (300 MHz, CDCl₃) δ 6.90-6.62 (m, 1H), 6.49 (m, 1H), 3.68-3.40 (m, 8H), 3.20-2.71 (m, 4H), 2.32 (t, J=7.6 Hz, 2H), 1.90-1.05 (m, 47H), 0.89 (t, J=6.7 Hz, 6H). ESI-MS for C₃₄H₆₄NO₆ ([M+H]⁺) Found 582.5.

BL13: ¹H NMR (400 MHz, CDCl₃) δ 6.96-6.74 (m, 1H), 6.74-6.61 (m, 1H), 6.61-6.33 (m, 1H), 3.77 (m, 7H), 3.00-2.54 (m, 8H), 2.01-1.75 (m, 6H), 1.43 (m, 50H), 0.90 (t, J=6.8 Hz, 6H).

BL14: ¹H NMR (400 MHz, CDCl₃) δ 7.20-6.34 (m, 3H), 3.90-3.63 (m, 6H), 3.18-2.73 (m, 4H), 1.98-1.07 (m, 49H), 0.90 (t, J=6.8 Hz, 6H).

BL15: ¹H NMR (400 MHz, CDCl₃) δ 6.60 (m, 3H), 4.85-4.51 (m, 3H), 4.16-3.91 (m, 3H), 3.84-3.67 (m, 5H), 3.67-3.52 (m, 4H), 3.50-3.32 (m, 4H), 2.69 (t, 9H), 2.20-1.03 (m, 56H), 0.97-0.72 (m, 6H).

BL16: ¹H NMR (400 MHz, CDCl₃) δ 8.39 (s, 1H), 4.09-3.98 (s, 3H), 3.62-3.51 (m, 2H), 2.76 (m, 10H), 2.50 (m, 8H), 1.62-1.04 (m, 84H), 0.90 (t, J=6.8 Hz, 12H). MALDI-MS for C₆₀H₁₁₇N₉O₂ ([M+H]⁺) Found 996.9.

BL17: ¹H NMR (300 MHz, CDCl₃) δ 8.40 (s, 1H), 4.05 (s, 3H), 3.66-3.38 (m, 2H), 2.68-2.30 (m, 6H), 1.77-1.12 (m, 53H), 0.88 (t, J=6.7 Hz, 6H). MALDI-MS for C₃₆H₆₇N₇O₂ ([M+H]⁺) Found 630.6.

BL18: ¹H NMR (400 MHz, CDCl₃) δ 8.40 (s, 1H), 4.05 (s, 3H), 3.66-3.38 (m, 2H), 2.68-2.30 (m, 6H), 1.77-1.12 (m, 60H), 0.88 (t, J=6.7 Hz, 6H).

BL19: ¹H NMR (300 MHz, CDCl₃) δ 8.41 (s, 1H), 4.67 (s, 4H), 4.06 (s, 3H), 3.57 (m, 14H), 2.98-2.42 (m, 7H), 1.86-1.12 (m, 38H), 0.90 (t, J=6.7 Hz, 6H). ESI-MS for C₃₈H₇₂N₇O₆ ([M+H]⁺) Found 722.7.

BL20: ¹H NMR (400 MHz, CDCl₃) δ 8.39 (s, 1H), 4.37-4.04 (m, 8H), 4.04 (s, 3H), 3.66-3.37 (m, 2H), 2.64-2.18 (m, 7H), 1.81-1.07 (m, 44H), 0.88 (t, J=6.8 Hz, 6H). ESI-MS for C₃₈H₆₈N₇O₈ ([M+H]⁺) Found 750.7.

BL21: ¹H NMR (400 MHz, CDCl₃) δ 8.40 (s, 1H), 4.63 (t, J=21.0 Hz, 2H), 4.15-3.87 (m, 4H), 3.70-3.26 (m, 16H), 2.72-2.29 (m, 8H), 1.81-1.02 (m, 65H), 0.89 (t, J=6.4 Hz, 6H). ESI-MS for C₄₄H₈₄N₇O₆ ([M+H⁺]+) Found 806.8.

BL22: ¹H NMR (300 MHz, CDCl₃) δ 8.46 (s, 1H), 4.10 (s, 3H), 3.89-3.52 (m, 4H), 2.85-2.30 (m, 13H), 2.18 (s, 3H), 1.93-1.04 (m, 64H), 0.89 (t, J=6.5 Hz, 6H). ESI-MS for C₃₇H₇₁N₈O₂ ([M+H]⁺) Found 659.7.

BL23: ¹H NMR (400 MHz, CDCl₃) δ 8.36 (s, 1H), 4.67 (s, 4H), 4.17 (s, 3H), 3.60-3.36 (m, 18H), 2.68-2.31 (m, 17H), 2.31-2.16 (m, 4H), 1.90-1.01 (m, 71H), 0.90 (t, J=6.7 Hz, 6H).

BL24: ¹H NMR (400 MHz, CDCl₃) δ 8.13 (s, 1H), 4.81-4.61 (s, 3H), 3.50 (m, 7H), 3.12-2.20 (m, 24H), 2.17 (s, 3H), 1.89-1.02 (m, 56H), 0.89 (t, J=6.8 Hz, 6H). ESI-MS for C₄₅H₈₇N₈O₂ ([M+H]⁺) Found 771.8.

BL25: ¹H NMR (300 MHz, CDCl₃) δ 4.20 (s, 1H), 4.00 (s, 2H), 3.31 (m, 8H), 3.05 (m, 10H), 1.46 (m, 93H), 0.89 (t, J=6.6 Hz, 12H). MALDI-MS for C₅₇H₁₂₀N₅O₂ ([M+H]⁺) Found 907.0.

BL26: ¹H NMR (400 MHz, CDCl₃) δ 4.25-3.82 (m, 2H), 3.78-3.60 (m, 1H), 3.28 (m, 2H), 2.50 (m, 6H), 1.74-1.09 (m, 46H), 0.90 (t, J=6.7 Hz, 6H). MALDI-MS for C₃₃H₇₀N₃O₂ ([M+H]⁺) Found 540.5.

BL27: ¹H NMR (400 MHz, CDCl₃) δ 4.71 (m, 3H), 4.30-4.01 (m, 3H), 3.99 (m, 2H), 3.74-3.44 (m, 13H), 3.39-3.17 (m, 12H), 2.42-1.11 (m, 80H), 1.03-0.79 (t, J=6.7 Hz, 9H). ESI-MS for C₄₉H₁₀₃N₄O₈ ([M+H]⁺) Found: 875.9.

BL28: ¹H NMR (400 MHz, CDCl₃) δ 4.13 (t, J=6.8 Hz, 12H), 3.96-3.78 (m, 1H), 3.78-3.57 (m, 1H), 3.49-3.24 (m, 3H), 2.52 (m, 14H), 1.85-1.06 (m, 76H), 0.90 (t, J=6.8 Hz, 9H). ESI-MS for C₅₈H₁₁₆N₄O₁₁ ([M+2H]²⁺) Found: 522.7.

BL29: ¹H NMR (400 MHz, CDCl₃) δ 4.14 (t, J=6.7 Hz, 8H), 3.97-3.83 (m, 1H), 3.80-3.61 (m, 1H), 3.33 (m, 3H), 2.66-2.38 (m, 6H), 1.78-1.10 (m, 57H), 0.90 (t, J=6.8 Hz, 6H). ESI-MS for C₄₃H₈₆N₃O₈ ([M+H]⁺) Found: 772.7.

BL30: ¹H NMR (400 MHz, CDCl₃) δ 4.68 (s, 4H), 3.88 (m, 1H), 3.78-3.60 (m, 1H), 3.53 (m, 8H), 3.35 (m, 3H), 2.65-2.33 (m, 5H), 1.83-1.09 (m, 52H), 0.90 (t, J=6.8 Hz, 6H). ESI-MS for C₄₃H₉₁N₃O₆ ([M+2H]²⁺) Found: 373.1.

BL31: ¹H NMR (400 MHz, CDCl₃) δ 4.68 (s, 4H), 3.94-3.82 (m, 1H), 3.77-3.68 (m, 1H), 3.67 (m, 4H), 3.60-3.49 (m, 4H), 3.45-3.16 (m, 3H), 2.81 (t, J=6.0 Hz, 4H), 2.68-2.51 (m, 2H), 1.69-1.07 (m, 40H), 0.90 (t, J=6.8 Hz, 6H). ESI-MS for C₃₅H₇₅N₃O₆ ([M+2H]²⁺) Found: 317.0.

BL32: ¹H NMR (300 MHz, CDCl₃) δ 7.26 (m, 4H), 6.69 (d, J=8.7 Hz, 4H), 6.45 (d, J=15.8 Hz, 2H), 6.24-5.99 (m, 2H), 3.27 (t, J=19.7 Hz, 4H), 2.94 (s, 12H), 2.71-2.30 (m, 13H), 1.35 (m, 59H), 0.90 (t, J=6.6 Hz, 9H). MALDI-MS for C₆₂H₁₁₂N₅ ([M+H]⁺) Found: 926.8.

BL33: ¹H NMR (400 MHz, CDCl₃) δ 7.29 (m, 4H), 6.67 (m, 4H), 6.50 (t, J=16.4 Hz, 2H), 6.27-6.02 (m, 2H), 3.45 (m, 4H), 2.98 (s, 12H), 2.67 (m, 8H), 1.90-1.09 (m, 45H), 0.90 (t, J=6.8 Hz, 6H). MALDI-MS for C₅₂H₉₁N₄ ([M+H]⁺) Found: 771.7.

BL34: ¹H NMR (300 MHz, CDCl₃) δ 7.53 (d, J=15.5 Hz, 1H), 7.37 (d, J=8.8 Hz, 2H), 6.71-6.51 (m, 2H), 6.23 (d, J=15.5 Hz, 1H), 4.23-3.99 (m, 8H), 3.44-3.23 (m, 2H), 2.97 (s, 6H), 2.67-2.37 (m, 4H), 1.76-1.09 (m, 38H), 0.86 (t, J=6.6 Hz, 6H). ESI-MS for C₄₃H₇₆N₃O₇ ([M+H]⁺) Found: 746.7.

BL35: ¹H NMR (400 MHz, CDCl₃) δ 7.53 (d, 1H), 7.41 (d, J=8.8 Hz, 2H), 6.70 (m, 2H), 6.21 (d, J=15.5 Hz, 1H), 4.68 (m, 2H), 3.64-3.54 (m, 4H), 3.46-3.35 (m, 6H), 3.02 (s, 6H), 1.75-1.07 (m, 56H), 0.89 (t, J=6.7 Hz, 6H). ESI-MS for C₄₃H₈₄N₃O₅ ([M+H]⁺) Found: 746.6.

BL36: ¹H NMR (400 MHz, CDCl₃) δ 7.56 (d, J=15.5 Hz, 1H), 7.40 (d, 2H), 6.77-6.55 (m, 2H), 6.19 (d, 1H), 4.66 (m, 4H), 3.62-3.45 (m, 8H), 3.38 (m, 2H), 3.09-2.93 (m, 6H), 2.42 (d, J=49.0 Hz, 6H), 1.71-1.04 (m, 49H), 0.89 (t, J=6.5 Hz, 6H). ESI-MS for C₄₇H₈₈N₃O₅ ([M+H]⁺) Found: 774.8.

BL37: ¹H NMR (400 MHz, CDCl₃) δ 7.65-7.45 (m, 1H), 7.41 (d, J=8.7 Hz, 2H), 6.75-6.54 (m, 2H), 6.25 (d, J=15.6 Hz, 1H), 3.12-2.93 (s, 6H), 2.68-2.31 (m, 14H), 1.64-1.05 (m, 59H), 0.97-0.75 (t, J=6.8 Hz, 9H). ESI-MS for C₅₁H₉₈N₄O ([M+2H]²⁺) Found: 391.7.

BL38: ¹H NMR (400 MHz, CDCl₃) δ 7.55 (d, J=15.5 Hz, 1H), 7.42 (d, J=8.5 Hz, 2H), 6.64 (d, 2H), 6.23 (d, J=15.3 Hz, 1H), 4.14 (m, 12H), 3.40 (d, J=19.8 Hz, 2H), 3.19-2.84 (m, 6H), 2.53 (dd, J=30.3, 23.1 Hz, 11H), 1.83-1.06 (m, 49H), 0.89 (t, J=6.5 Hz, 9H). ESI-MS for C₅₄H₉₈N₄O₁₀ ([M+2H]²⁺) Found: 481.7.

BL39: ¹H NMR (400 MHz, CDCl₃) δ 7.55 (d, J=7.5 Hz, 1H), 7.42 (d, J=7.9 Hz, 2H), 6.70 (d, J=11.3 Hz, 2H), 6.21 (d, J=15.3 Hz, 1H), 4.74-4.53 (m, 3H), 3.67-3.26 (m, 14H), 3.16-2.88 (m, 6H), 2.54 (m, 8H), 1.74-1.08 (m, 52H), 0.90 (t, J=6.6 Hz, 9H). ESI-MS for C₅₄H₉₈N₄O₁₀ ([M+2H]²⁺) Found: 481.7.

BL40: ¹H NMR (400 MHz, CDCl₃) δ 7.55 (d, J=15.5 Hz, 1H), 7.43 (d, J=14.7 Hz, 2H), 6.65 (d, J=21.3 Hz, 2H), 6.22 (d, J=15.5 Hz, 1H), 4.86-4.54 (m, 6H), 3.89-3.13 (m, 17H), 3.07-2.89 (m, 6H), 2.82-2.54 (m, 4H), 1.43 (m, 55H), 0.96-0.77 (t, 9H). ESI-MS for C₅₄H₁₀₄N₄O₇ ([M+2H]²⁺) Found: 460.5.

BL41: ¹H NMR (400 MHz, CDCl₃) δ 7.52 (d, 1H), 7.40 (d, J=8.7 Hz, 2H), 6.70 (d, J=12.0 Hz, 2H), 6.22 (d, J=14.3 Hz, 1H), 4.11 (m, 11H), 3.12-2.80 (m, 6H), 2.48 (dd, J=86.3, 57.9 Hz, 11H), 1.79-1.06 (m, 66H), 0.89 (t, J=6.8 Hz, 9H). ESI-MS for C₆₆H₁₂₂N₄O₁₀ ([M+2H]²⁺) Found: 565.8.

BL42: ¹H NMR (400 MHz, CDCl₃) δ 7.68-7.47 (d, 1H), 7.41 (d, J=8.8 Hz, 2H), 6.68 (d, J=8.8 Hz, 2H), 6.24 (d, J=15.5 Hz, 1H), 3.83-3.49 (m, 2H), 3.46-3.24 (m, 2H), 3.01 (s, 6H), 2.64 (m, 6H), 1.74-1.00 (m, 63H), 0.98-0.74 (t, 6H). ESI-MS for C₄₉H₉₃N₃O ([M+2H]²⁺) Found: 370.2.

BL43: ¹H NMR (400 MHz, CDCl₃) δ 7.53 (t, J=19.4 Hz, 1H), 7.40 (t, J=12.5 Hz, 2H), 6.80-6.59 (m, 2H), 6.25 (d, J=15.5 Hz, 1H), 3.76-3.55 (m, 2H), 3.47-3.32 (m, 3H), 3.05-2.97 (s, 6H), 1.77-1.11 (m, 72H), 0.90 (t, J=6.8 Hz, 6H). ESI-MS for C₄₅H₈₅N₃O ([M+2H]²⁺) Found: 342.1.

BL44: ¹H NMR (400 MHz, CDCl₃) δ 7.58-7.45 (d, 1H), 7.39 (d, J=8.7 Hz, 2H), 6.68 (d, J=8.8 Hz, 2H), 6.29-6.10 (d, 1H), 4.76-4.53 (m, 2H), 3.68-3.52 (m, 4H), 3.47-3.29 (m, 6H), 2.97 (d, J=19.4 Hz, 6H), 2.61-2.30 (m, 13H), 2.30-2.17 (m, 4H), 1.84-1.15 (m, 55H), 0.88 (t, J=6.8, 6H). ESI-MS for C₅₀H₉₅N₄O₅ ([M+H]⁺) Found: 831.8.

BL45: ¹H NMR (400 MHz, CDCl₃) δ 7.63-7.47 (d, 1H), 7.39 (d, J=14.1 Hz, 2H), 6.70 (d, 2H), 6.22 (d, J=15.6 Hz, 1H), 3.58-3.35 (m, 2H), 3.08-2.95 (m, 5H), 2.62-2.34 (m, 1H), 2.34-2.15 (m, 5H), 1.84-1.11 (m, 45H), 0.90 (t, J=6.8 Hz, 6H). ESI-MS for C₄₂H₇₉N₄O ([M+H]⁺) Found: 655.8.

BL46: ¹H NMR (400 MHz, CDCl₃) δ 7.48 (d, J=14.3 Hz, 1H), 7.39 (d, J=8.8 Hz, 2H), 6.76-6.55 (d, 2H), 6.20 (d, J=15.6 Hz, 1H), 4.66 (s, 4H), 3.59-3.36 (m, 10H), 2.97 (s, 6H), 2.61-2.30 (m, 13H), 2.30-2.12 (s, 3H), 1.82-1.13 (m, 37H), 0.89 (t, J=6.8 Hz, 6H). ESI-MS for C₄₄H₈₃N₄O₅ ([M+H⁺]+) Found: 747.8.

BL47: ¹H NMR (400 MHz, CDCl₃) δ 7.57-7.44 (d, 1H), 7.41 (d, 2H), 6.75-6.62 (d, 2H), 6.20 (d, J=15.6 Hz, 1H), 4.28-3.99 (m, 8H), 3.60-3.39 (m, 2H), 2.99 (s, 6H), 2.58-2.38 (m, 9H), 2.29 (s, 3H), 1.80-1.17 (m, 36H), 0.89 (t, J=6.8 Hz, 6H). ESI-MS for C₄₄H₇₉N₄O₇ ([M+H]⁺) Found: 775.7.

BL48: ¹H NMR (400 MHz, CDCl₃) δ 7.53 (d, 1H), 7.39 (d, J=12.7 Hz, 2H), 6.73-6.56 (d, 2H), 6.22 (d, J=15.5 Hz, 1H), 4.90-4.75 (m, 2H), 3.35 (m, 2H), 3.05-2.97 (m, 6H), 2.40-2.20 (m, 6H), 1.80-1.11 (m, 61H), 1.02-0.70 (t, 12H). ESI-MS for C₅₁H₉₃N₃O₅ ([M+2H]²⁺) Found: 414.2.

BL49: ¹H NMR (400 MHz, CDCl₃) δ 7.53 (d, J=15.5 Hz, 1H), 7.40 (d, J=12.8 Hz, 2H), 6.70 (d, J=8.8 Hz, 2H), 6.21 (d, J=14.5 Hz, 1H), 4.92-4.72 (m, 2H), 4.14 (m, 2H), 3.61-3.34 (m, 4H), 3.02 (s, 6H), 2.68-2.16 (m, 17H), 2.07 (s, 3H), 1.84-1.08 (m, 50H), 0.97-0.78 (t, 12H). ESI-MS for C₅₂H₉₅N₄O₅ ([M+H]⁺) Found: 855.9.

BL50: ¹H NMR (400 MHz, CDCl₃) δ 7.62 (d, J=7.8 Hz, 1H), 7.38 (d, J=8.1 Hz, 1H), 7.17 (m, 2H), 7.04 (d, 1H), 4.36-3.94 (m, 8H), 3.30-2.99 (m, 4H), 2.68-2.26 (m, 8H), 1.87-1.08 (m, 43H), 0.90 (t, J=6.7 Hz, 6H). ESI-MS for C₄₃H₇₄N₃O₇ ([M+H]⁺) Found:744.7.

BL51: ¹H NMR (400 MHz, CDCl₃) δ 7.69-7.52 (d, 1H), 7.37 (d, J=12.3 Hz, 1H), 7.25-7.06 (m, 2H), 7.02 (d, J=8.4 Hz, 1H), 4.68 (s, 4H), 3.54 (m, 8H), 3.28-3.03 (m, 4H), 2.66-2.35 (m, 8H), 1.70-1.03 (m, 42H), 0.90 (t, J=6.8 Hz, 6H). ESI-MS for C₄₃H₇₈N₃O₅ ([M+H]⁺) Found:716.7.

BL52: ¹H NMR (400 MHz, CDCl₃) δ 7.69-7.49 (m, 1H), 7.35 (d, J=8.0 Hz, 1H), 7.22-7.04 (m, 2H), 7.01 (d, J=14.0 Hz, 1H), 4.80-4.54 (m, 2H), 3.69-3.52 (m, 4H), 3.48-3.33 (m, 4H), 3.24-3.03 (m, 4H), 2.68-2.30 (m, 8H), 1.68-1.04 (m, 49H), 0.89 (t, J=6.7 Hz, 6H). ESI-MS for C₄₅H₈₂N₃O₅ ([M+H]⁺) Found:744.8.

BL53: ¹H NMR (400 MHz, CDCl₃) δ 7.59 (d, J=16.0 Hz, 1H), 7.34 (d, J=9.8 Hz, 1H), 7.13 (m, 2H), 7.04 (d, J=1.5 Hz, 1H), 4.12 (m, 12H), 3.21 (m, 4H), 2.70-2.21 (m, 14H), 1.82-1.11 (m, 47H), 0.89 (t, J=6.7 Hz, 9H). ESI-MS for C₅₄H₉₆N₄O₁₀ ([M+2H]²⁺) Found:480.7.

BL54: ¹H NMR (400 MHz, CDCl₃) δ 7.57 (d, 1H), 7.37 (d, J=10.4 Hz, 1H), 7.23-6.96 (m, 3H), 4.66 (m, 3H), 3.72-3.30 (m, 13H), 3.30-3.07 (m, 4H), 2.72-2.23 (m, 13H), 1.97-1.01 (m, 59H), 0.89 (t, J=6.7 Hz, 9H). ESI-MS for C₅₇H₁₀₇N₄O₇ ([M+H]⁺) Found:959.9.

BL55: ¹H NMR (400 MHz, CDCl₃) δ 7.70-7.44 (m, 1H), 7.37 (d, 1H), 7.18-6.93 (m, 2H), 3.10 (t, J=6.5 Hz, 2H), 2.89-2.63 (m, 4H), 2.57 (m, 2H), 1.70-0.95 (m, 44H), 0.92-0.77 (t, 6H). ESI-MS for C₄₉H₉₀N₃O ([M+H]⁺) Found:736.8.

BL56: ¹H NMR (400 MHz, CDCl₃) δ 7.58 (d, J=7.6 Hz, 1H), 7.35 (d, J=8.1 Hz, 1H), 7.21-7.06 (m, 2H), 7.02 (t, J=7.2 Hz, 1H), 3.21-3.06 (m, 4H), 2.77-2.27 (m, 9H), 1.82-1.04 (m, 64H), 0.90 (t, J=6.8 Hz, 6H). ESI-MS for C₄₅H₈₂N₃O ([M+H]⁺) Found:680.7.

BL57: ¹H NMR (400 MHz, CDCl₃) δ 7.62 (d, J=7.8 Hz, 1H), 7.49-7.31 (m, 1H), 7.16 (m, 2H), 7.03 (t, J=10.9 Hz, 1H), 3.41-3.21 (m, 2H), 3.06 (m, 2H), 2.71-2.41 (m, 8H), 2.38-2.19 (m, 4H), 2.19-1.98 (m, 3H), 1.77-1.09 (m, 45H), 0.90 (t, J=6.8 Hz, 6H). ESI-MS for C₄₂H₇₇N₄O ([M+H]⁺) Found:653.7.

BL58: ¹H NMR (400 MHz, CDCl₃) δ 7.59 (d, 1H), 7.47-7.29 (d, 1H), 7.13 (m, 2H), 7.04 (t, 1H), 4.68 (s, 4H), 3.67-3.44 (m, 8H), 3.23 (m, 2H), 2.67-1.95 (m, 15H), 1.79-1.16 (m, 33H), 0.91 (t, J=6.8 Hz, 6H). ESI-MS for C₄₄H₈₁N₄O₅ ([M+H]⁺) Found:745.8.

BL59: ¹H NMR (300 MHz, CDCl₃) δ 7.60 (d, J=7.7 Hz, 1H), 7.34 (d, J=10.5 Hz, 1H), 7.24-7.04 (m, 2H), 7.03 (d, J=1.8 Hz, 1H), 4.26-4.00 (m, 8H), 3.28 (m, 2H), 3.12 (m, 2H), 2.68-2.51 (m, 2H), 2.50-2.20 (m, 11H), 2.12 (s, 3H), 1.75-1.08 (m, 40H), 0.89 (t, J=6.6 Hz, 6H). ESI-MS for C₄₄H₇₇N₄O₇ ([M+H]⁺) Found:773.7.

BL60: ¹H NMR (300 MHz, CDCl₃) δ 7.58 (d, 1H), 7.34 (d, J=7.8 Hz, 1H), 7.15 (m, 2H), 7.04 (s, 1H), 4.75-4.55 (m, 2H), 3.70-3.01 (m, 12H), 2.67-2.15 (m, 14H), 2.14-1.99 (m, 4H), 1.79-1.12 (m, 50H), 0.89 (t, J=6.7 Hz, 6H). ESI-MS for C₅₀H₉₃N₄O₅ ([M+H]⁺) Found: 829.9.

BL61: ¹H NMR (300 MHz, CDCl₃) δ 7.61 (d, J=7.7 Hz, 1H), 7.42-7.30 (m, 1H), 7.23-7.07 (m, 2H), 7.03 (d, J=2.2 Hz, 1H), 4.67 (s, 4H), 4.13 (m, 2H), 3.63-3.37 (m, 9H), 3.22 (m, 4H), 2.69-2.19 (m, 14H), 2.08 (d, J=9.9 Hz, 3H), 2.06 (s, 3H), 1.72-1.05 (m, 51H), 0.89 (t, J=6.7 Hz, 6H). ESI-M S for C₄₈H₈₉N₄O₅ ([M+H]⁺) Found: 801.8.

BL62: ¹H NMR (300 MHz, CDCl₃) δ 7.62 (d, J=7.7 Hz, 1H), 7.43-7.32 (m, 1H), 7.15 (m, 2H), 7.04 (d, J=2.1 Hz, 1H), 3.39-3.18 (m, 2H), 3.13 (t, J=7.2 Hz, 2H), 2.71-2.36 (m, 9H), 2.36-2.17 (m, 4H), 2.08 (s, 3H), 1.78-1.08 (m, 61H), 0.93-0.84 (t, 6H). ESI-MS for C₄₆H₈₅N₄O ([M+H]⁺) Found: 709.8.

BL63: ¹H NMR (300 MHz, CDCl₃) δ 7.62 (d, J=7.8 Hz, 1H), 7.43-7.31 (m, 1H), 7.23-7.08 (m, 2H), 7.05 (d, J=2.1 Hz, 1H), 3.27 (m, 2H), 3.13 (t, J=7.2 Hz, 2H), 2.68-2.37 (m, 9H), 2.35-2.14 (m, 4H), 2.16-1.96 (m, 4H), 1.74-1.06 (m, 61H), 0.90 (t, J=6.7 Hz, 6H). ESI-MS for C₅₀H₉₃N₄O ([M+H]⁺) Found: 765.9.

BL64: ¹H NMR (400 MHz, CDCl₃) δ 7.60 (d, J=13.8 Hz, 1H), 7.37 (d, J=8.1 Hz, 1H), 7.24-7.09 (m, 2H), 7.04 (t, J=6.0 Hz, 1H), 4.95-4.67 (m, 2H), 3.29-3.01 (m, 4H), 2.69-2.51 (m, 3H), 2.51-2.20 (m, 10H), 1.80-1.04 (m, 60H), 1.02-0.79 (m, 12H). ESI-MS for C₅₁H₉₀N₃O₅ ([M+H]⁺) Found: 824.8.

BL65: ¹H NMR (400 MHz, CDCl₃) δ 7.42-7.31 (m, 4H), 7.16-6.96 (m, 2H), 6.92-6.73 (m, 1H), 3.37-3.14 (m, 2H), 2.43 (m, 9H), 2.23-2.13 (m, 2H), 1.93-1.10 (m, 54H), 0.89 (t, J=6.8 Hz, 6H).

BL66: ¹H NMR (300 MHz, CDCl₃) δ 7.44-7.29 (m, 4H), 7.09-6.95 (m, 2H), 6.83 (m, 1H), 4.66 (s, 4H), 3.51 (m, 9H), 3.36-3.14 (m, 2H), 2.82-2.11 (m, 12H), 1.91-1.05 (m, 51H), 0.89 (t, J=6.8 Hz, 6H). ESI-MS for C₅₃H₈₈ClF₂N₂O₇S ([M+H]⁺) Found: 969.8.

BL67; ¹H NMR (300 MHz, CDCl₃) δ 7.46-7.29 (m, 4H), 7.17-6.94 (m, 2H), 6.96-6.66 (m, 1H), 3.25 (m, 2H), 2.74 (m, 4H), 2.51-2.27 (m, 4H), 2.31-2.11 (m, 2H), 1.94-1.00 (m, 65H), 0.89 (t, J=6.7 Hz, 6H). ESI-MS for C₅₉H₁₀₀ClF₂N₂O₃S ([M+H]⁺) Found: 989.9.

BL68: ¹H NMR (300 MHz, CDCl₃) δ 7.42-7.30 (m, 4H), 7.16-6.96 (m, 2H), 6.92-6.70 (m, 1H), 4.31-3.93 (m, 9H), 3.36-3.09 (m, 2H), 2.62-2.08 (m, 13H), 1.94-1.17 (m, 53H), 0.89 (t, J=6.7 Hz, 6H). ESI-MS for C₅₃H₈₄ClF₂N₂O₉S ([M+H]⁺) Found: 997.8.

BL69: ¹H NMR (400 MHz, CDCl₃) δ 7.34 (m, 4H), 7.11-6.94 (m, 2H), 6.83 (m, 1H), 3.94-3.60 (m, 4H), 3.37-3.11 (m, 2H), 2.57-2.25 (m, 10H), 2.25-2.08 (m, 2H), 1.95-1.03 (m, 71H), 0.89 (t, J=6.8 Hz, 6H). ESI-MS for C₅₅H₉₂ClF₂N₂O₃S ([M+H]⁺) Found: 933.8.

BL70: ¹H NMR (400 MHz, CDCl₃) δ 7.47-7.31 (m, 4H), 7.11-6.95 (m, 2H), 6.92-6.74 (m, 1H), 4.75-4.57 (m, 2H), 3.68-3.52 (m, 4H), 3.46-3.31 (m, 4H), 3.31-3.13 (m, 2H), 2.67-1.98 (m, 13H), 1.87-1.06 (m, 58H), 0.89 (t, J=6.8 Hz, 6H). ESI-MS for C₅₉H₁₀₀ClF₂N₂O₇S ([M+H]⁺) Found: 1053.7.

BL71; ¹H NMR (400 MHz, CDCl₃) δ 7.43-7.31 (m, 4H), 7.12-7.01 (m, 2H), 6.89-6.76 (m, 1H), 4.92-4.69 (m, 2H), 3.45-3.22 (m, 2H), 2.58-2.06 (m, 23H), 1.88-1.16 (m, 56H), 1.00-0.79 (t, 12H). ESI-MS for C₆₂H₁₀₃ClF₂N₃O₇S ([M+H]⁺) Found: 1106.8.

BL72: ¹H NMR (400 MHz, CDCl₃) δ 7.41-7.29 (m, 4H), 7.06 (m, 2H), 6.84 (m, 1H), 3.39-3.23 (m, 2H), 2.62-2.31 (m, 12H), 2.16 (m, 2H), 1.91-1.00 (m, 58H), 1.00-0.74 (t, 6H). ESI-MS for C₅₂H₈₇ClF₂N₃O₃S ([M+H]⁺) Found: 906.8.

BL73: ¹H NMR (400 MHz, CDCl₃) δ 7.48-7.27 (m, 4H), 7.14-7.00 (m, 3H), 6.88-6.73 (m, 1H), 3.45-3.27 (m, 2H), 2.63-2.30 (m, 12H), 2.19 (m, 2H), 1.88-1.09 (m, 53H), 0.90 (t, J=6.8 Hz, 6H). ESI-MS for C₅₄H₈₇ClF₂N₃O₉S ([M+H]⁺) Found: 962.9.

BL74; ¹H NMR (400 MHz, CDCl₃) δ 7.48-7.31 (m, 4H), 7.06 (m, 2H), 6.91-6.68 (m, 1H), 3.43-3.15 (m, 2H), 2.43 (m, 12H), 2.21-2.02 (m, 2H), 1.95-1.01 (m, 62H), 0.90 (t, J=6.8 Hz, 6H). ESI-MS for C₆₀H₁₀₃ClF₂N₃O₃S ([M+H]⁺) Found: 1018.8.

BL75: ¹H NMR (300 MHz, CDCl₃) δ 7.49-7.30 (m, 4H), 7.06 (m, 2H), 6.92-6.74 (m, 1H), 4.14 (m, 8H), 3.44-3.23 (m, 2H), 2.58-2.03 (m, 16H), 1.90-1.06 (m, 40H), 0.90 (t, J=6.6 Hz, 6H). ESI-MS for C₅₄H₈₇ClF₂N₃O₉S ([M+H]⁺) Found: 1026.7.

BL76: ¹H NMR (300 MHz, CDCl₃) δ 7.46-7.27 (m, 4H), 7.13-6.96 (m, 2H), 6.89-6.74 (m, 1H), 4.67 (s, 4H), 3.68-3.45 (m, 8H), 3.32 (m, 2H), 2.63-2.30 (m, 14H), 2.20 (s, 3H), 1.88-1.14 (m, 45H), 0.90 (t, J=6.8 Hz, 6H). ESI-MS for C₅₄H₉₁ClF₂N₃O₇S ([M+H]⁺) Found: 998.8.

BL77: ¹H NMR (400 MHz, CDCl₃) δ 2.64-2.31 (m, 4H), 2.24-2.05 (m, 2H), 1.69-1.04 (m, 53H), 0.89 (m, 12H). ESI-MS for C₃₆H₇₀N ([M+H⁺]) Found: 516.8.

BL78: ¹H NMR (400 MHz, CDCl₃) δ 2.66-2.47 (m, 4H), 2.23-2.04 (m, 2H), 1.58-0.99 (m, 65H), 0.94-0.77 (m, 12H). ESI-MS for C₄₄H₈₆N ([M+H]⁺) Found: 628.8.

BL79: ¹H NMR (300 MHz, CDCl₃) δ 7.37 (dd, J=8.3, 7.5 Hz, 1H), 6.53 (dd, J=15.4, 7.9 Hz, 2H), 2.91-2.64 (m, 4H), 2.57-2.29 (m, 5H), 1.97-1.03 (m, 58H), 0.90 (t, J=6.7 Hz, 6H). ESI-MS for C₃₄H₆₃ClN₃ ([M+H]⁺) Found: 548.5.

BL80: ¹H NMR (300 MHz, CDCl₃) δ 7.44-7.35 (m, 1H), 6.54 (m, 2H), 4.17 (m, 10H), 3.06-2.83 (m, 4H), 2.83-2.53 (m, 5H), 2.09-1.01 (m, 40H), 0.89 (t, J=6.7 Hz, 6H). ESI-MS for C₃₆H₆₃ClN₃O₆ ([M+H]⁺) Found: 668.5.

BL81: ¹H NMR (400 MHz, CDCl₃) δ 7.32 (dd, J=16.4, 8.6 Hz, 1H), 6.56-6.40 (m, 2H), 4.63 (d, J=1.6 Hz, 4H), 3.60-3.28 (m, 8H), 2.86-2.61 (m, 4H), 2.54-2.24 (m, 5H), 1.88-1.18 (m, 33H), 0.87 (t, J=6.8 Hz, 6H). ESI-MS for C₃₆H₆₇C₁N₃O₄ ([M+H]⁺) Found: 640.6.

BL82: ¹H NMR (400 MHz, CDCl₃) δ 7.41 (dd, J=14.6, 7.6 Hz, 1H), 6.56 (m, 2H), 4.14 (m, 8H), 3.76 (m, 6H), 3.01-2.84 (m, 4H), 2.84-2.55 (m, 5H), 2.06-1.09 (m, 43H), 0.91 (t, J=6.9 Hz, 6H).

BL83: ¹H NMR (300 MHz, CDCl₃) δ 7.45-7.31 (m, 1H), 6.53 (dd, J=14.9, 7.9 Hz, 2H), 2.76 (m, 4H), 2.62-2.33 (m, 5H), 1.35 (m, 57H), 0.89 (t, J=6.7 Hz, 6H).

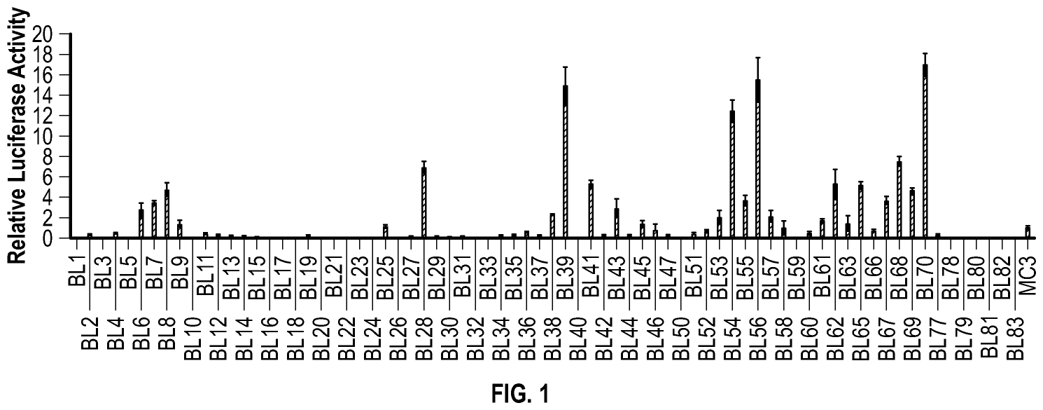

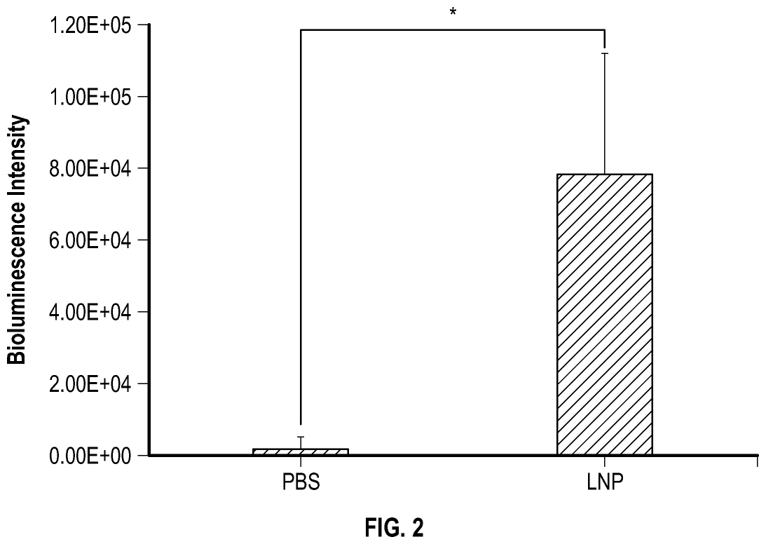

Lipid nanoparticles comprising compounds BL1-BL83, depicted in Table 1, were independently formed to encapsulate firefly luciferase mRNA for screening of activity in CCL131 cells. MC3 LNP normalized data of the screened particles is shown in FIG. 1. A cluster-testing method was used to examine mRNA delivery in vivo using several of the brain targeting lipids depicted in Table 1 (BL28, BL39, BL54, BL68 and BL70) against a PBS-treated group. Comparative bioluminescence intensity of the groups is shown in FIG. 2.

TABLE 1

| | |
|---|---|
| BL1 | 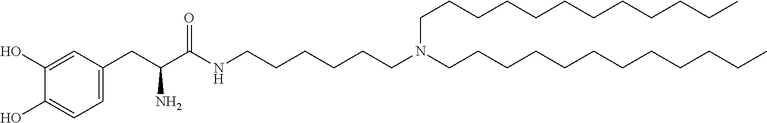 [SMILES: CCCCCCCCCCCCN(CCCCCCCCCCCC)CCCCCCNC(=O)[C@@H](N)Cc1ccc(O)c(O)c1] |
| BL2 | 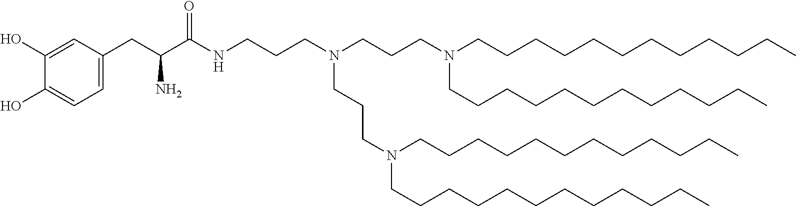 [SMILES: CCCCCCCCCCCCN(CCCCCCCCCCCC)CCCN(CCCNC(=O)[C@@H](N)Cc1ccc(O)c(O)c1)CCCN(CCCCCCCCCCCC)CCCCCCCCCCCC] |
| BL3 | 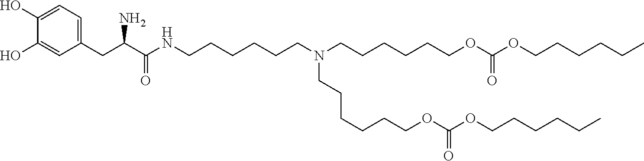 [SMILES: CCCCCCOC(=O)OCCCCCCN(CCCCCCNC(=O)[C@H](N)Cc1ccc(O)c(O)c1)CCCCCCOC(=O)OCCCCCC] |
| BL4 | 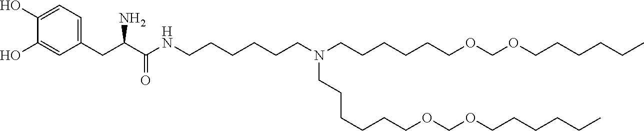 [SMILES: CCCCCCOCOCCCCCCN(CCCCCCNC(=O)[C@H](N)Cc1ccc(O)c(O)c1)CCCCCCOCOCCCCCC] |
| BL5 | 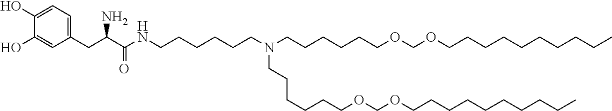 [SMILES: CCCCCCCCCCOCOCCCCCCN(CCCCCCNC(=O)[C@H](N)Cc1ccc(O)c(O)c1)CCCCCCOCOCCCCCCCCCC] |
| BL6 | 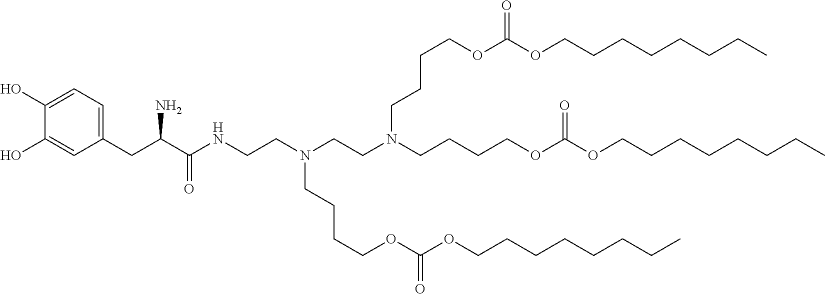 [SMILES: CCCCCCCCOC(=O)OCCCCN(CCCCOC(=O)OCCCCCCCC)CCN(CCCCOC(=O)OCCCCCCCC)CCNC(=O)[C@H](N)Cc1ccc(O)c(O)c1] |
| BL7 | 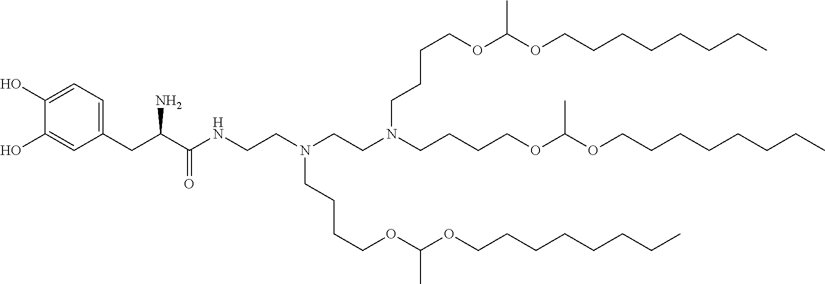 [SMILES: CCCCCCCCOC(C)OCCCCN(CCCCOC(C)OCCCCCCCC)CCN(CCCCOC(C)OCCCCCCCC)CCNC(=O)[C@H](N)Cc1ccc(O)c(O)c1] |
| BL8 | 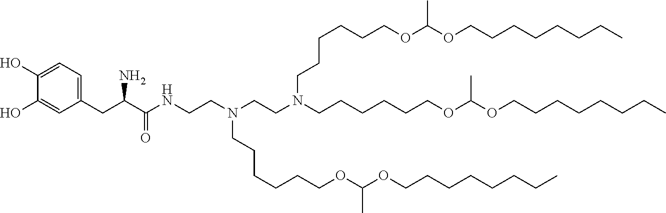 [SMILES: CCCCCCCCOC(C)OCCCCCCN(CCCCCCOC(C)OCCCCCCCC)CCN(CCCCCCOC(C)OCCCCCCCC)CCNC(=O)[C@H](N)Cc1ccc(O)c(O)c1] |
| BL9 | 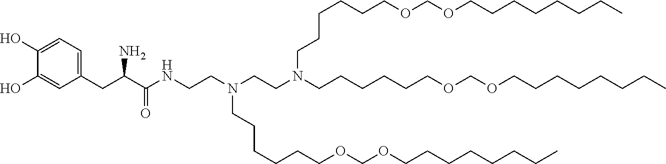 [SMILES: CCCCCCCCOCOCCCCCCN(CCCCCCOCOCCCCCCCC)CCN(CCCCCCOCOCCCCCCCC)CCNC(=O)[C@H](N)Cc1ccc(O)c(O)c1] |
| BL10 | 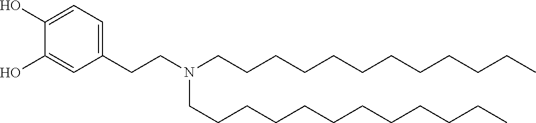 [SMILES: CCCCCCCCCCCCN(CCCCCCCCCCCC)CCc1ccc(O)c(O)c1] |
| BL11 | 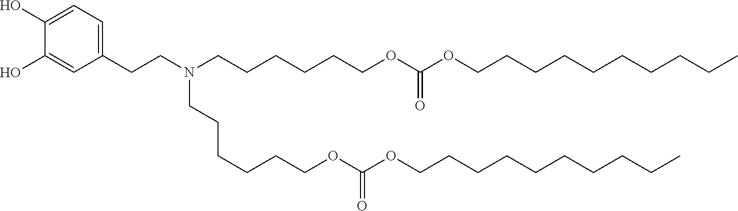 [SMILES: CCCCCCCCCCOC(=O)OCCCCCCN(CCCCCCOC(=O)OCCCCCCCCCC)CCc1ccc(O)c(O)c1] |
| BL12 | 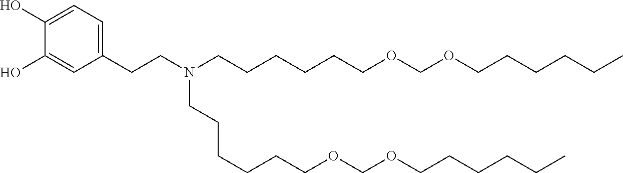 [SMILES: CCCCCCOCOCCCCCCN(CCCCCCOCOCCCCCC)CCc1ccc(O)c(O)c1] |
| BL13 | 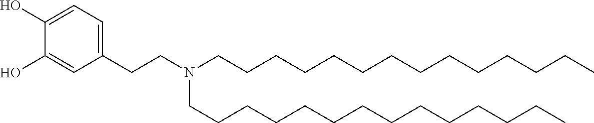 [SMILES: CCCCCCCCCCCCCCN(CCCCCCCCCCCCCC)CCc1ccc(O)c(O)c1] |
| BL14 | 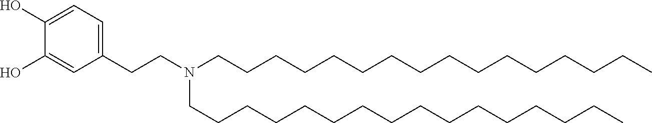 [SMILES: CCCCCCCCCCCCCCCCN(CCCCCCCCCCCCCCCC)CCc1ccc(O)c(O)c1] |
| BL15 | 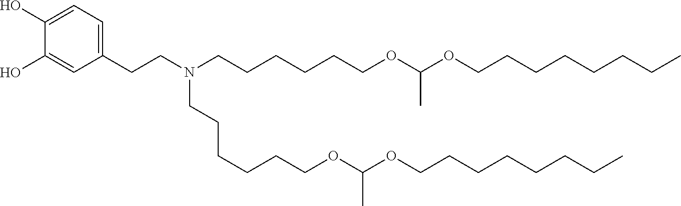 [SMILES: CCCCCCCCOC(C)OCCCCCCN(CCCCCCOC(C)OCCCCCCCC)CCc1ccc(O)c(O)c1] |
| BL16 | 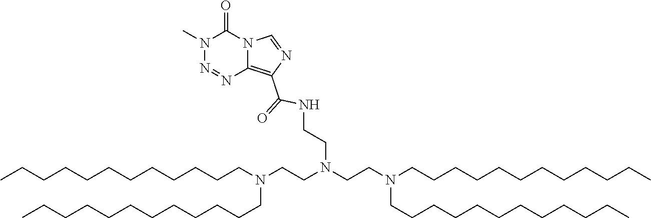 [SMILES: CCCCCCCCCCCCN(CCCCCCCCCCCC)CCN(CCNC(=O)c1ncn2c(=O)n(C)nnc12)CCN(CCCCCCCCCCCC)CCCCCCCCCCCC] |
| BL17 | 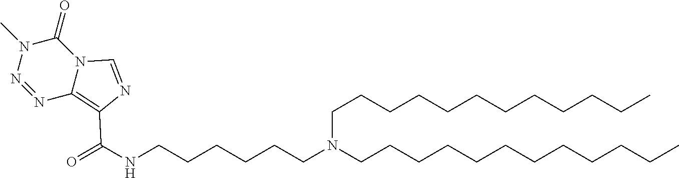 [SMILES: CCCCCCCCCCCCN(CCCCCCCCCCCC)CCCCCCNC(=O)c1ncn2c(=O)n(C)nnc12] |
| BL18 | 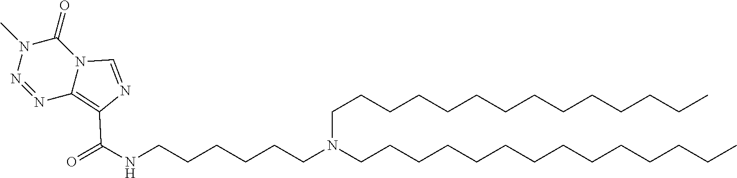 [SMILES: CCCCCCCCCCCCCCN(CCCCCCCCCCCCCC)CCCCCCNC(=O)c1ncn2c(=O)n(C)nnc12] |
| BL19 | 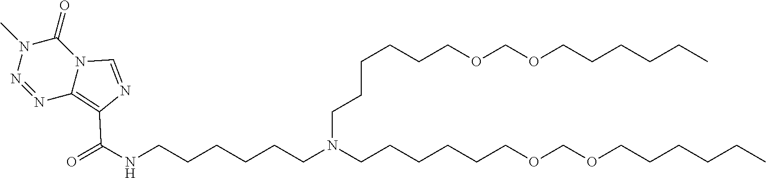 [SMILES: CCCCCCOCOCCCCCCN(CCCCCCNC(=O)c1ncn2c(=O)n(C)nnc12)CCCCCCOCOCCCCCC] |
| BL20 | 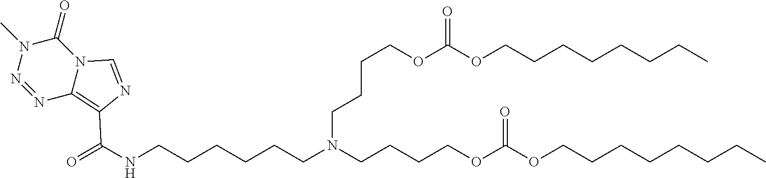 [SMILES: CCCCCCCCOC(=O)OCCCCN(CCCCCCNC(=O)c1ncn2c(=O)n(C)nnc12)CCCCOC(=O)OCCCCCCCC] |
| BL21 | 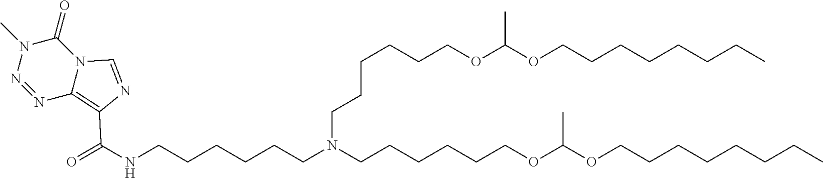 [SMILES: CCCCCCCCOC(C)OCCCCCCN(CCCCCCNC(=O)c1ncn2c(=O)n(C)nnc12)CCCCCCOC(C)OCCCCCCCC] |
| BL22 | 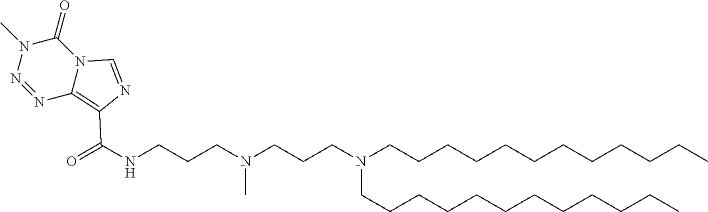 [SMILES: CCCCCCCCCCCCN(CCCCCCCCCCCC)CCCN(C)CCCNC(=O)c1ncn2c(=O)n(C)nnc12] |
| BL23 | 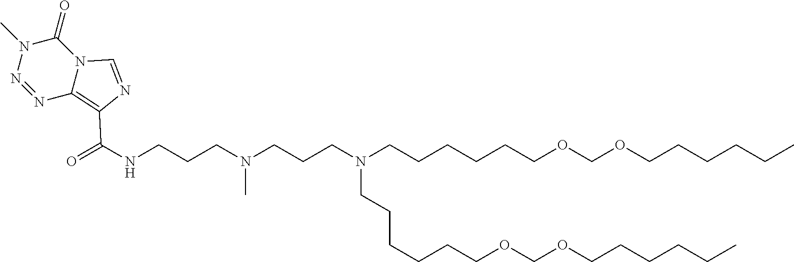 [SMILES: CCCCCCOCOCCCCCCN(CCCCCCOCOCCCCCC)CCCN(C)CCCNC(=O)c1ncn2c(=O)n(C)nnc12] |
| BL24 | 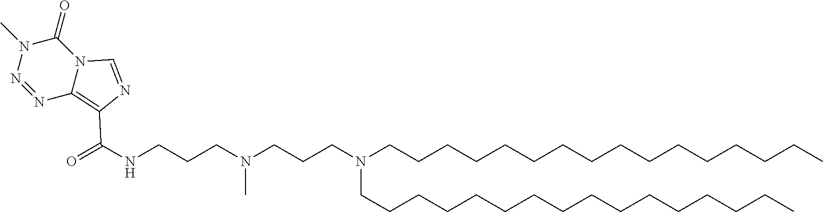 [SMILES: CCCCCCCCCCCCCCCCN(CCCCCCCCCCCCCCCC)CCCN(C)CCCNC(=O)c1ncn2c(=O)n(C)nnc12] |
| BL25 | 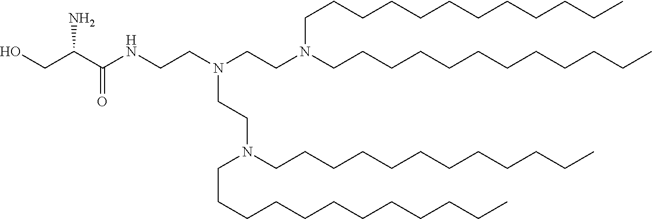 [SMILES: CCCCCCCCCCCCN(CCCCCCCCCCCC)CCN(CCNC(=O)[C@@H](N)CO)CCN(CCCCCCCCCCCC)CCCCCCCCCCCC] |
| BL26 | 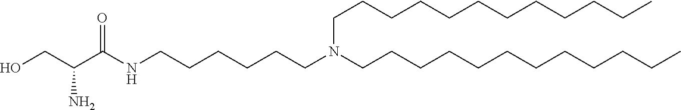 [SMILES: CCCCCCCCCCCCN(CCCCCCCCCCCC)CCCCCCNC(=O)[C@H](N)CO] |
| BL27 | 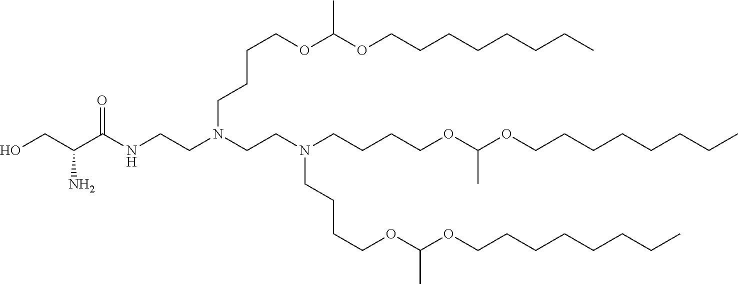 [SMILES: CCCCCCCCOC(C)OCCCCN(CCCCOC(C)OCCCCCCCC)CCN(CCCCOC(C)OCCCCCCCC)CCNC(=O)[C@H](N)CO] |
| BL28 | 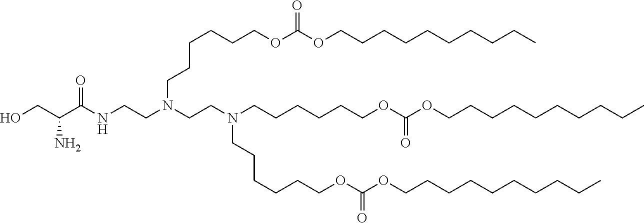 [SMILES: CCCCCCCCCCOC(=O)OCCCCCCN(CCCCCCOC(=O)OCCCCCCCCCC)CCN(CCCCCCOC(=O)OCCCCCCCCCC)CCNC(=O)[C@H](N)CO] |
| BL29 | 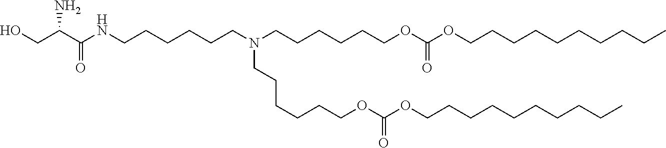 [SMILES: CCCCCCCCCCOC(=O)OCCCCCCN(CCCCCCNC(=O)[C@@H](N)CO)CCCCCCOC(=O)OCCCCCCCCCC] |
| BL30 | 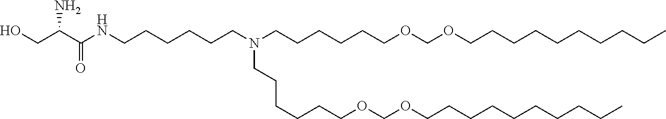 [SMILES: CCCCCCCCCCOCOCCCCCCN(CCCCCCNC(=O)[C@@H](N)CO)CCCCCCOCOCCCCCCCCCC] |
| BL31 | 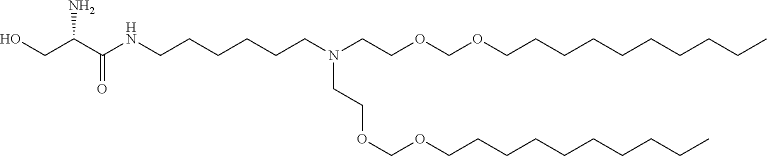 [SMILES: CCCCCCCCCCOCOCCN(CCCCCCNC(=O)[C@@H](N)CO)CCOCOCCCCCCCCCC] |
| BL32 | 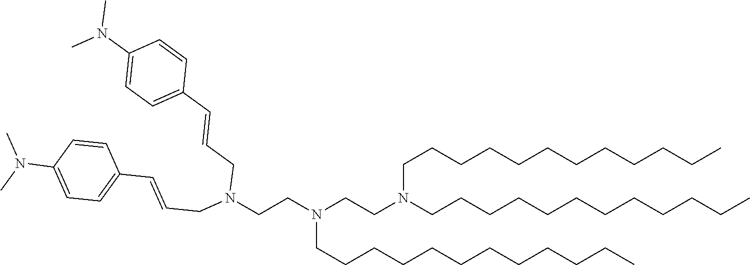 [SMILES: CCCCCCCCCCCCN(CCCCCCCCCCCC)CCN(CCCCCCCCCCCC)CCN(C/C=C/c1ccc(N(C)C)cc1)C/C=C/c1ccc(N(C)C)cc1] |
| BL33 | 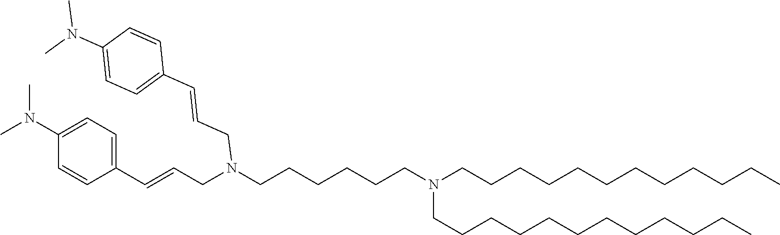 [SMILES: CCCCCCCCCCCCN(CCCCCCCCCCCC)CCCCCCN(C/C=C/c1ccc(N(C)C)cc1)C/C=C/c1ccc(N(C)C)cc1] |
| BL34 | 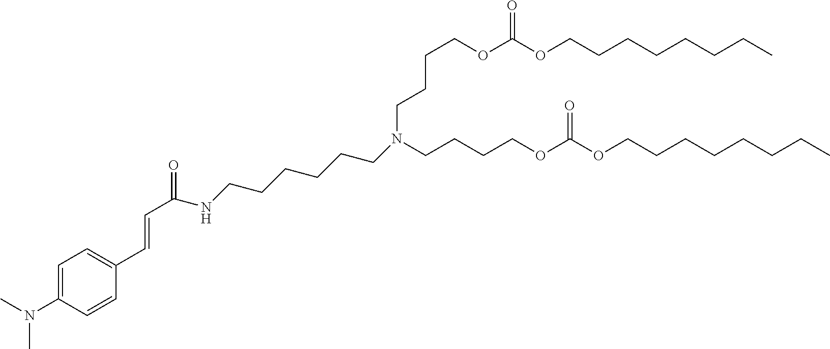 [SMILES: CCCCCCCCOC(=O)OCCCCN(CCCCCCNC(=O)/C=C/c1ccc(N(C)C)cc1)CCCCOC(=O)OCCCCCCCC] |
| BL35 | 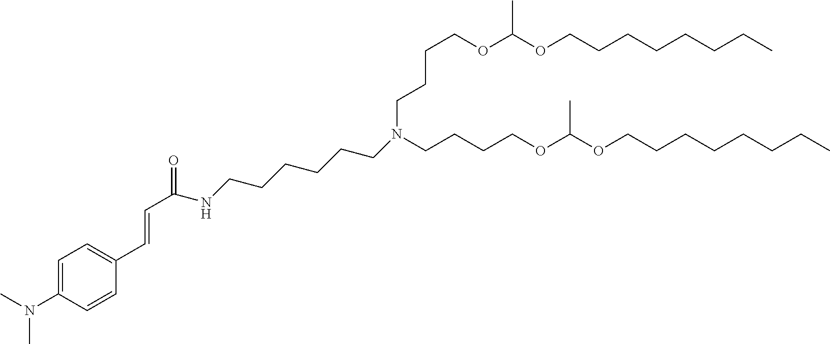 [SMILES: CCCCCCCCOC(C)OCCCCN(CCCCCCNC(=O)/C=C/c1ccc(N(C)C)cc1)CCCCOC(C)OCCCCCCCC] |
| BL36 | 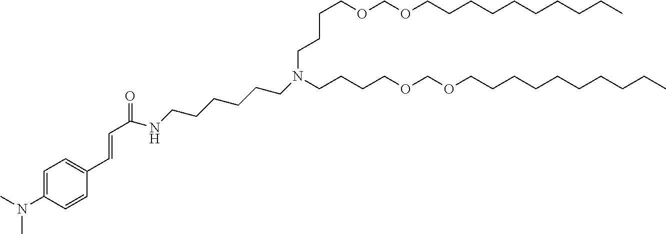 [SMILES: CCCCCCCCCCOCOCCCCN(CCCCCCNC(=O)/C=C/c1ccc(N(C)C)cc1)CCCCOCOCCCCCCCCCC] |
| BL37 | 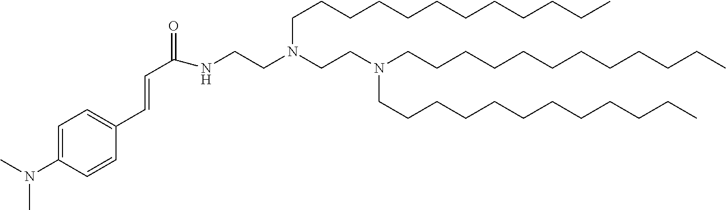 [SMILES: CCCCCCCCCCCCN(CCCCCCCCCCCC)CCN(CCCCCCCCCCCC)CCNC(=O)/C=C/c1ccc(N(C)C)cc1] |
| BL38 | 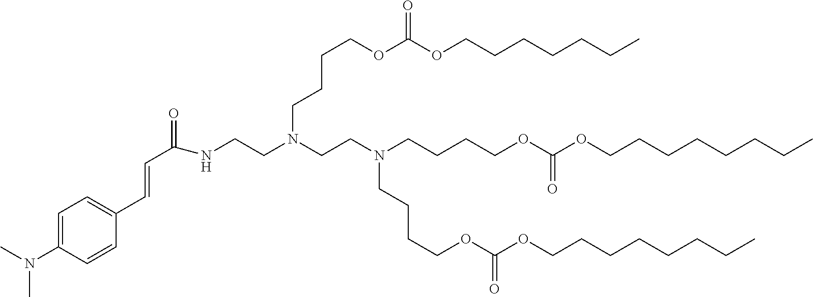 [SMILES: CCCCCCCCOC(=O)OCCCCN(CCCCOC(=O)OCCCCCCCC)CCN(CCCCOC(=O)OCCCCCCC)CCNC(=O)/C=C/c1ccc(N(C)C)cc1] |
| BL39 | 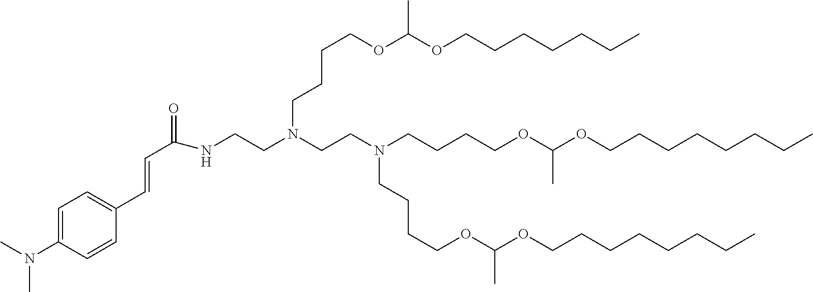 [SMILES: CCCCCCCCOC(C)OCCCCN(CCCCOC(C)OCCCCCCCC)CCN(CCCCOC(C)OCCCCCCC)CCNC(=O)/C=C/c1ccc(N(C)C)cc1] |
| BL40 | 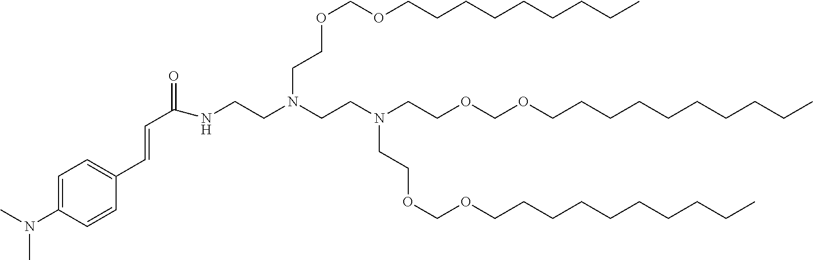 [SMILES: CCCCCCCCCCOCOCCN(CCOCOCCCCCCCCCC)CCN(CCNC(=O)/C=C/c1ccc(N(C)C)cc1)CCOCOCCCCCCCCC] |
| BL41 | 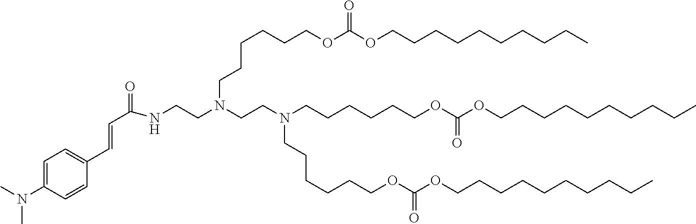 [SMILES: CCCCCCCCCCOC(=O)OCCCCCCN(CCCCCCOC(=O)OCCCCCCCCCC)CCN(CCCCCCOC(=O)OCCCCCCCCCC)CCNC(=O)/C=C/c1ccc(N(C)C)cc1] |
| BL42 | 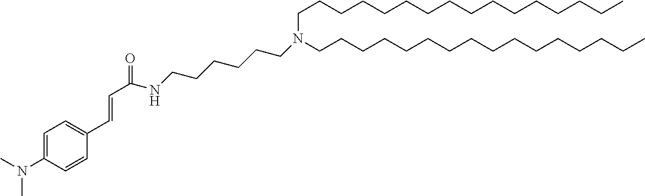 [SMILES: CCCCCCCCCCCCCCCCN(CCCCCCCCCCCCCCCC)CCCCCCNC(=O)/C=C/c1ccc(N(C)C)cc1] |
| BL43 | 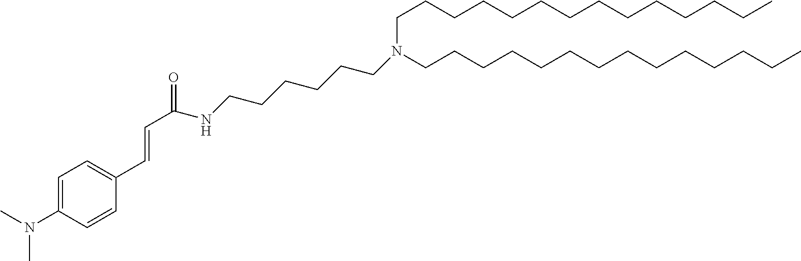 [SMILES: CCCCCCCCCCCCCCN(CCCCCCCCCCCCCC)CCCCCCNC(=O)/C=C/c1ccc(N(C)C)cc1] |
| BL44 | 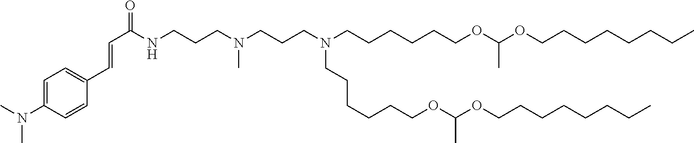 [SMILES: CCCCCCCCOC(C)OCCCCCCN(CCCCCCOC(C)OCCCCCCCC)CCCN(C)CCCNC(=O)/C=C/c1ccc(N(C)C)cc1] |
| BL45 | 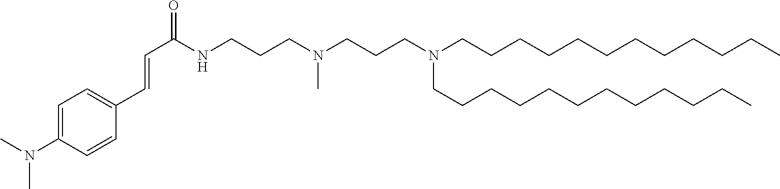 [SMILES: CCCCCCCCCCCCN(CCCCCCCCCCCC)CCCN(C)CCCNC(=O)/C=C/c1ccc(N(C)C)cc1] |
| BL46 | 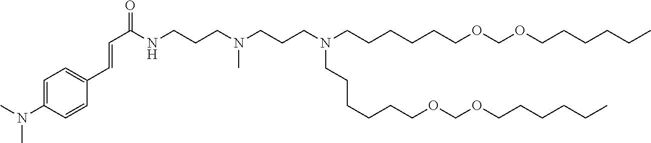 [SMILES: CCCCCCOCOCCCCCCN(CCCCCCOCOCCCCCC)CCCN(C)CCCNC(=O)/C=C/c1ccc(N(C)C)cc1] |
| BL47 | 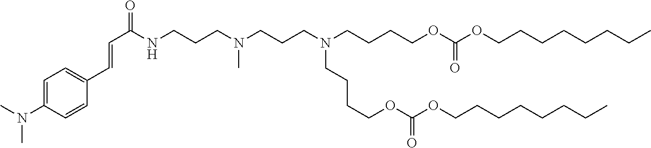 [SMILES: CCCCCCCCOC(=O)OCCCCN(CCCCOC(=O)OCCCCCCCC)CCCN(C)CCCNC(=O)/C=C/c1ccc(N(C)C)cc1] |
| BL48 | 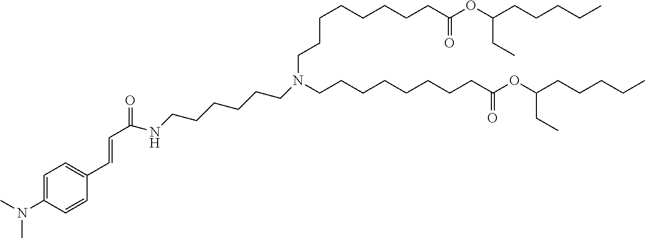 [SMILES: CCCCCC(CC)OC(=O)CCCCCCCCN(CCCCCCCCC(=O)OC(CC)CCCCC)CCCCCCNC(=O)/C=C/c1ccc(N(C)C)cc1] |
| BL49 | 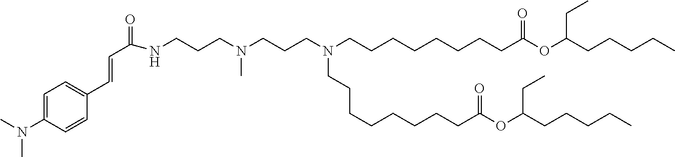 [SMILES: CCCCCC(CC)OC(=O)CCCCCCCCN(CCCCCCCCC(=O)OC(CC)CCCCC)CCCN(C)CCCNC(=O)/C=C/c1ccc(N(C)C)cc1] |
| BL50 | 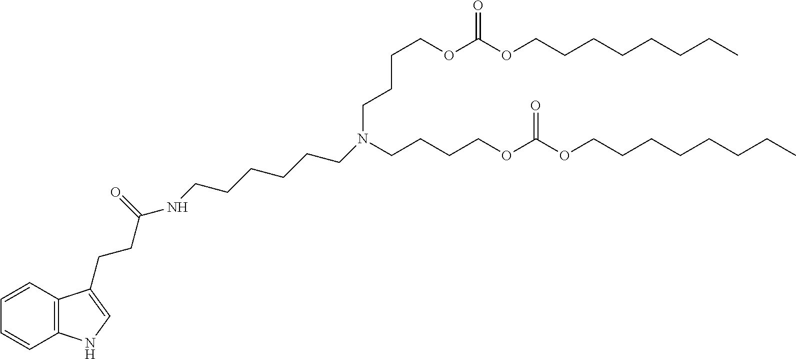 [SMILES: CCCCCCCCOC(=O)OCCCCN(CCCCCCNC(=O)CCc1c[nH]c2ccccc12)CCCCOC(=O)OCCCCCCCC] |
| BL51 | 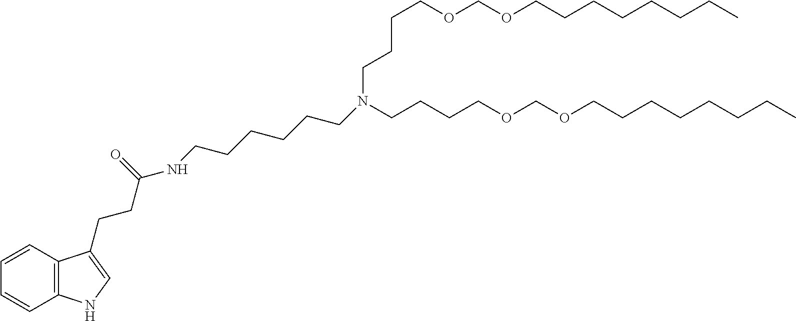 [SMILES: CCCCCCCCOCOCCCCN(CCCCCCNC(=O)CCc1c[nH]c2ccccc12)CCCCOCOCCCCCCCC] |
| BL52 | 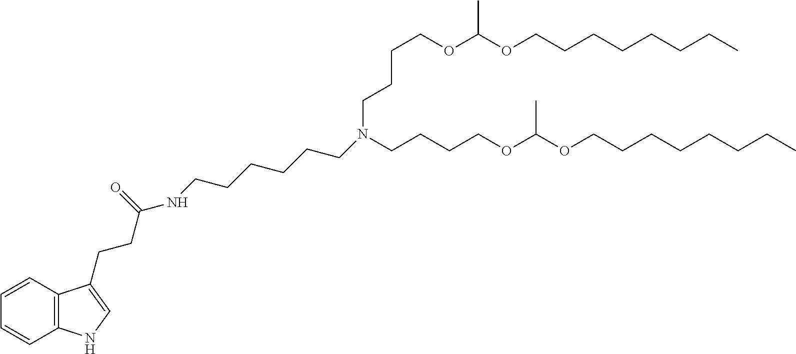 [SMILES: CCCCCCCCOC(C)OCCCCN(CCCCCCNC(=O)CCc1c[nH]c2ccccc12)CCCCOC(C)OCCCCCCCC] |
| BL53 | 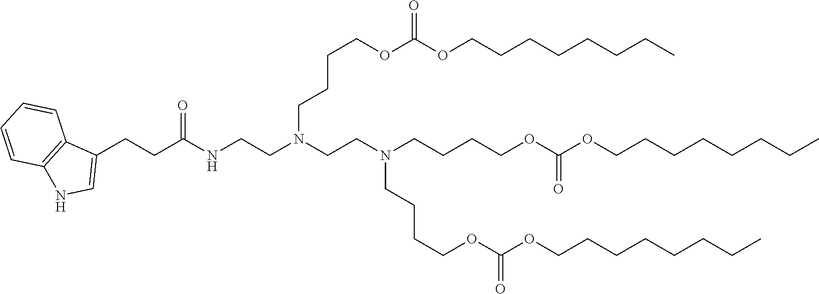 [SMILES: CCCCCCCCOC(=O)OCCCCN(CCCCOC(=O)OCCCCCCCC)CCN(CCCCOC(=O)OCCCCCCCC)CCNC(=O)CCc1c[nH]c2ccccc12] |
| BL54 | 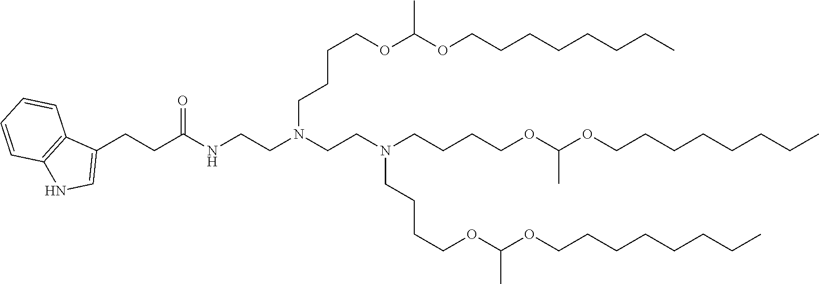 [SMILES: CCCCCCCCOC(C)OCCCCN(CCCCOC(C)OCCCCCCCC)CCN(CCCCOC(C)OCCCCCCCC)CCNC(=O)CCc1c[nH]c2ccccc12] |
| BL55 | 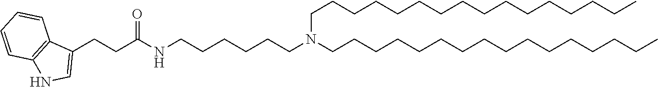 [SMILES: CCCCCCCCCCCCCCCCN(CCCCCCCCCCCCCCCC)CCCCCCNC(=O)CCc1c[nH]c2ccccc12] |
| BL56 | 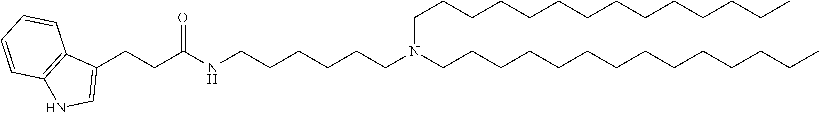 [SMILES: CCCCCCCCCCCCCCN(CCCCCCCCCCCCCC)CCCCCCNC(=O)CCc1c[nH]c2ccccc12] |
| BL57 | 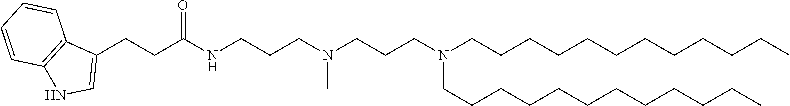 [SMILES: CCCCCCCCCCCCN(CCCCCCCCCCCC)CCCN(C)CCCNC(=O)CCc1c[nH]c2ccccc12] |
| BL58 | 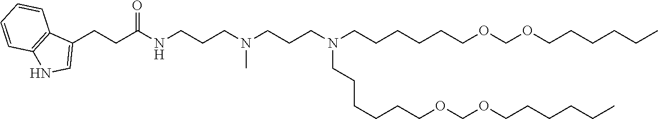 [SMILES: CCCCCCOCOCCCCCCN(CCCCCCOCOCCCCCC)CCCN(C)CCCNC(=O)CCc1c[nH]c2ccccc12] |
| BL59 | 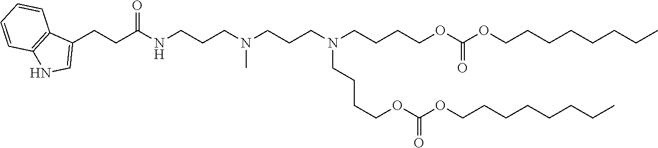 [SMILES: CCCCCCCCOC(=O)OCCCCN(CCCCOC(=O)OCCCCCCCC)CCCN(C)CCCNC(=O)CCc1c[nH]c2ccccc12] |
| BL60 | 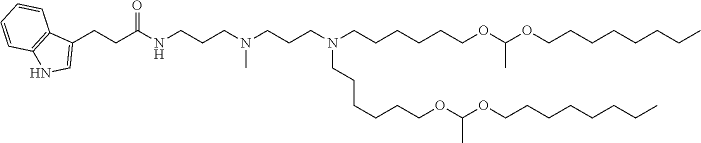 [SMILES: CCCCCCCCOC(C)OCCCCCCN(CCCCCCOC(C)OCCCCCCCC)CCCN(C)CCCNC(=O)CCc1c[nH]c2ccccc12] |
| BL61 | 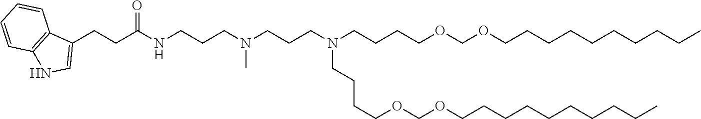 [SMILES: CCCCCCCCCCOCOCCCCN(CCCCOCOCCCCCCCCCC)CCCN(C)CCCNC(=O)CCc1c[nH]c2ccccc12] |
| BL62 | 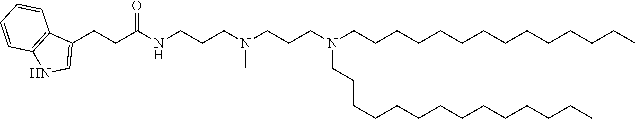 [SMILES: CCCCCCCCCCCCCCN(CCCCCCCCCCCCCC)CCCN(C)CCCNC(=O)CCc1c[nH]c2ccccc12] |
| BL63 | 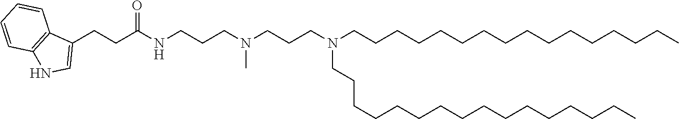 [SMILES: CCCCCCCCCCCCCCCCN(CCCCCCCCCCCCCCCC)CCCN(C)CCCNC(=O)CCc1c[nH]c2ccccc12] |
| BL64 | 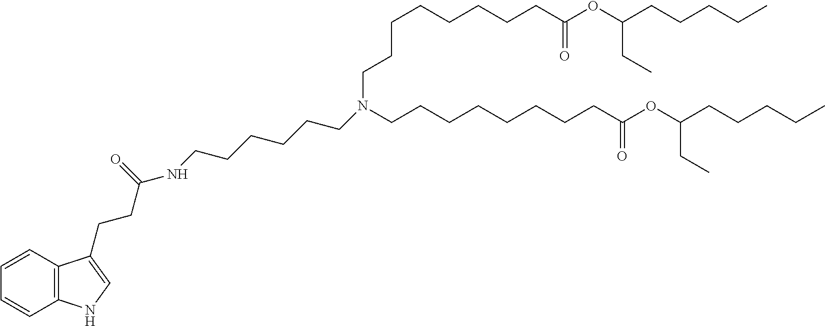 [SMILES: CCCCCC(CC)OC(=O)CCCCCCCCN(CCCCCCCCC(=O)OC(CC)CCCCC)CCCCCCNC(=O)CCc1c[nH]c2ccccc12] |
| BL65 | 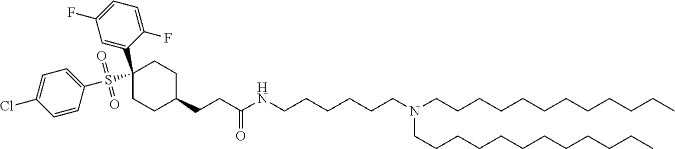 [SMILES: CCCCCCCCCCCCN(CCCCCCCCCCCC)CCCCCCNC(=O)CC[C@H]1CC[C@](c2cc(F)ccc2F)(S(=O)(=O)c2ccc(Cl)cc2)CC1] |
| BL66 | 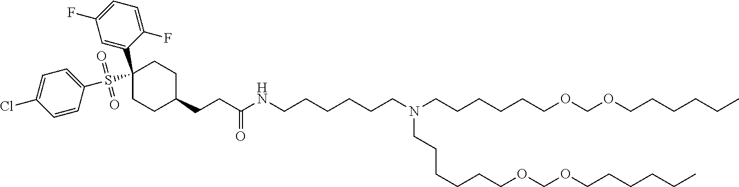 [SMILES: CCCCCCOCOCCCCCCN(CCCCCCNC(=O)CC[C@H]1CC[C@](c2cc(F)ccc2F)(S(=O)(=O)c2ccc(Cl)cc2)CC1)CCCCCCOCOCCCCCC] |
| BL67 | 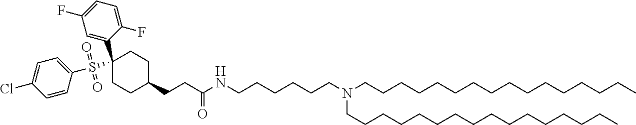 [SMILES: CCCCCCCCCCCCCCCCN(CCCCCCCCCCCCCCCC)CCCCCCNC(=O)CC[C@H]1CC[C@](c2cc(F)ccc2F)(S(=O)(=O)c2ccc(Cl)cc2)CC1] |
| BL68 | 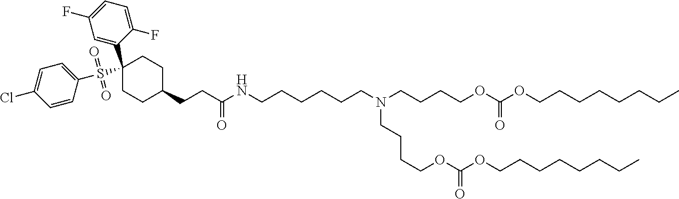 [SMILES: CCCCCCCCOC(=O)OCCCCN(CCCCCCNC(=O)CC[C@H]1CC[C@](c2cc(F)ccc2F)(S(=O)(=O)c2ccc(Cl)cc2)CC1)CCCCOC(=O)OCCCCCCCC] |
| BL69 | 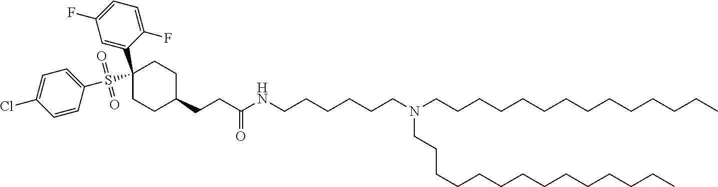 [SMILES: CCCCCCCCCCCCCCN(CCCCCCCCCCCCCC)CCCCCCNC(=O)CC[C@H]1CC[C@](c2cc(F)ccc2F)(S(=O)(=O)c2ccc(Cl)cc2)CC1] |
| BL70 | 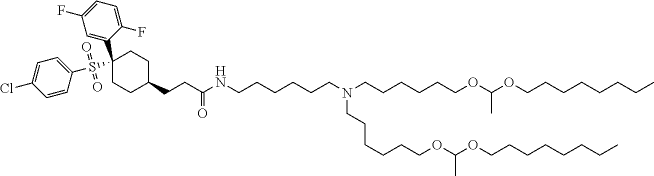 [SMILES: CCCCCCCCOC(C)OCCCCCCN(CCCCCCNC(=O)CC[C@H]1CC[C@](c2cc(F)ccc2F)(S(=O)(=O)c2ccc(Cl)cc2)CC1)CCCCCCOC(C)OCCCCCCCC] |
| BL71 | 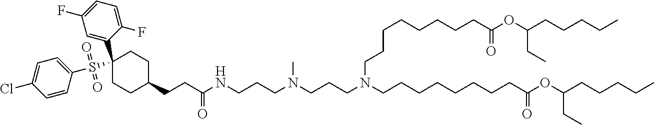 [SMILES: CCCCCC(CC)OC(=O)CCCCCCCCN(CCCCCCCCC(=O)OC(CC)CCCCC)CCCN(C)CCCNC(=O)CC[C@H]1CC[C@](c2cc(F)ccc2F)(S(=O)(=O)c2ccc(Cl)cc2)CC1] |
| BL72 | 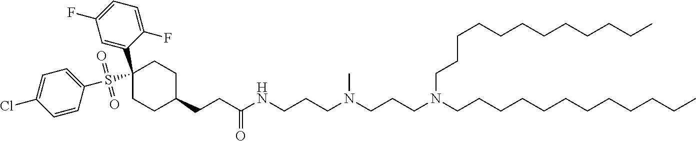 [SMILES: CCCCCCCCCCCCN(CCCCCCCCCCCC)CCCN(C)CCCNC(=O)CC[C@H]1CC[C@](c2cc(F)ccc2F)(S(=O)(=O)c2ccc(Cl)cc2)CC1] |
| BL73 | 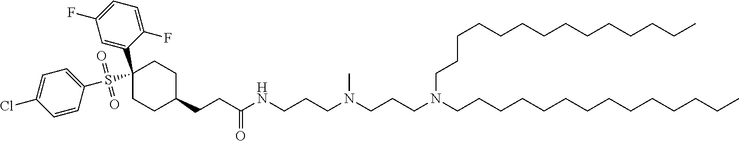 [SMILES: CCCCCCCCCCCCCCN(CCCCCCCCCCCCCC)CCCN(C)CCCNC(=O)CC[C@H]1CC[C@](c2cc(F)ccc2F)(S(=O)(=O)c2ccc(Cl)cc2)CC1] |
| BL74 | 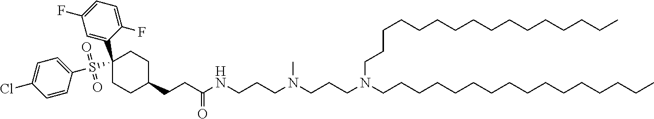 [SMILES: CCCCCCCCCCCCCCCCN(CCCCCCCCCCCCCCCC)CCCN(C)CCCNC(=O)CC[C@H]1CC[C@](c2cc(F)ccc2F)(S(=O)(=O)c2ccc(Cl)cc2)CC1] |
| BL75 | 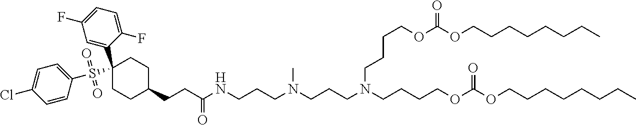 [SMILES: CCCCCCCCOC(=O)OCCCCN(CCCCOC(=O)OCCCCCCCC)CCCN(C)CCCNC(=O)CC[C@H]1CC[C@](c2cc(F)ccc2F)(S(=O)(=O)c2ccc(Cl)cc2)CC1] |
| BL76 | 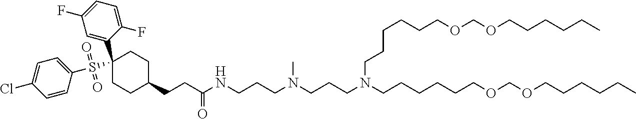 [SMILES: CCCCCCOCOCCCCCCN(CCCCCCOCOCCCCCC)CCCN(C)CCCNC(=O)CC[C@H]1CC[C@](c2cc(F)ccc2F)(S(=O)(=O)c2ccc(Cl)cc2)CC1] |
| BL77 | 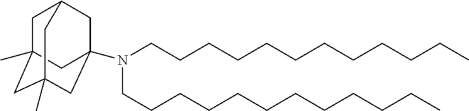 [SMILES: CCCCCCCCCCCCN(CCCCCCCCCCCC)C12CC3CC(C)(CC(C)(C3)C1)C2] |
| BL78 | 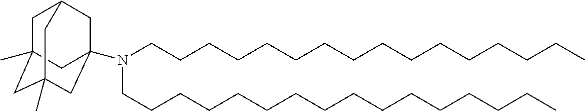 [SMILES: CCCCCCCCCCCCCCCCN(CCCCCCCCCCCCCCCC)C12CC3CC(C)(CC(C)(C3)C1)C2] |
| BL79 | 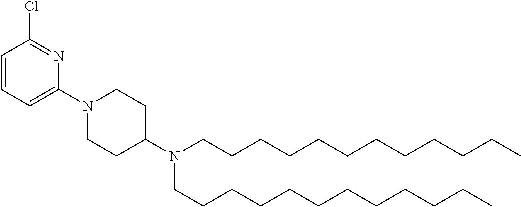 [SMILES: CCCCCCCCCCCCN(CCCCCCCCCCCC)C1CCN(c2cccc(Cl)n2)CC1] |
| BL80 | 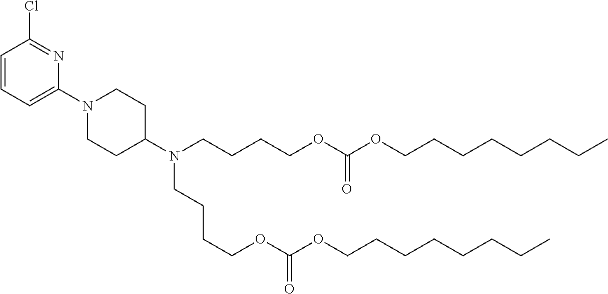 [SMILES: CCCCCCCCOC(=O)OCCCCN(CCCCOC(=O)OCCCCCCCC)C1CCN(c2cccc(Cl)n2)CC1] |
| BL81 | 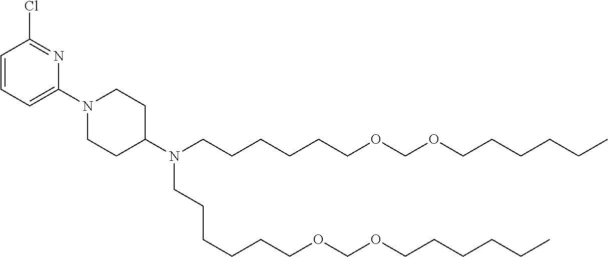 [SMILES: CCCCCCOCOCCCCCCN(CCCCCCOCOCCCCCC)C1CCN(c2cccc(Cl)n2)CC1] |
| BL82 | 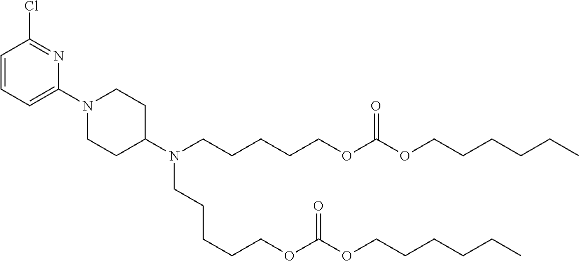 [SMILES: CCCCCCOC(=O)OCCCCCN(CCCCCOC(=O)OCCCCCC)C1CCN(c2cccc(Cl)n2)CC1] |
| BL83 | 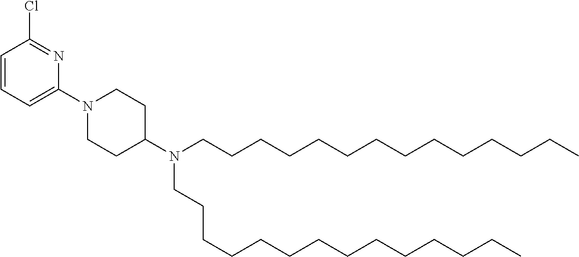 [SMILES: CCCCCCCCCCCCCCN(CCCCCCCCCCCCCC)C1CCN(c2cccc(Cl)n2)CC1] |

1. A compound represented by any one of Formulas I through XIX, or a pharmaceutically acceptable salt thereof: 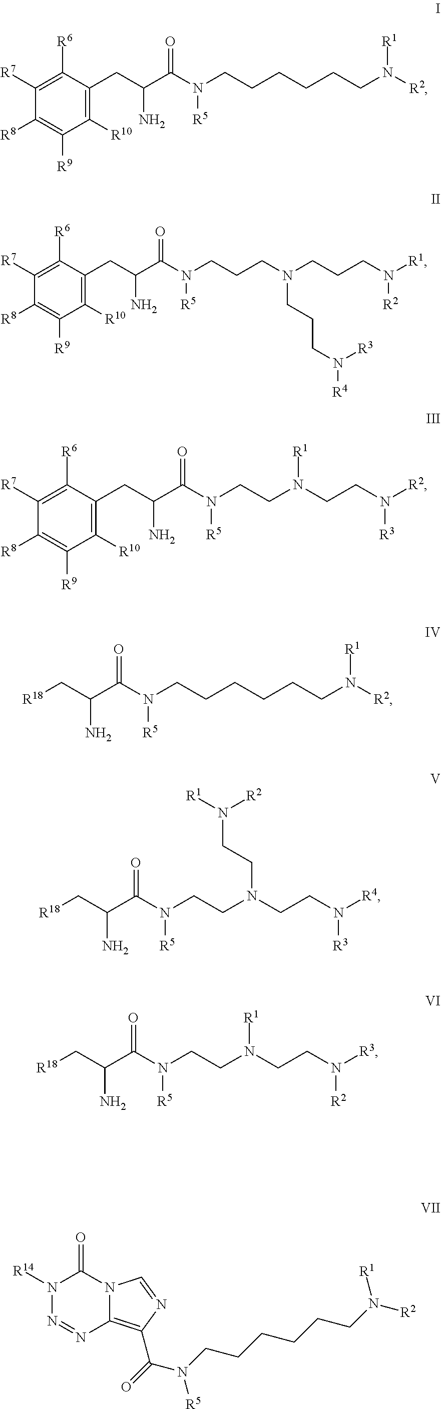 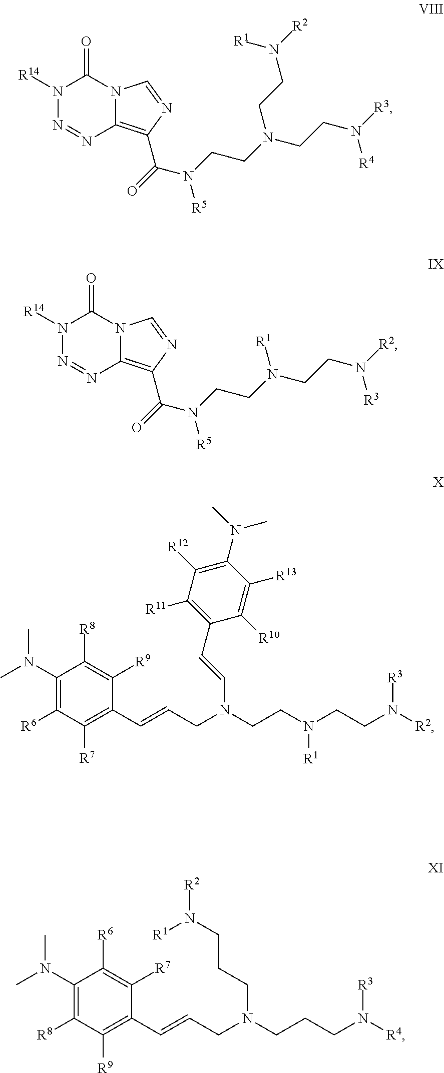 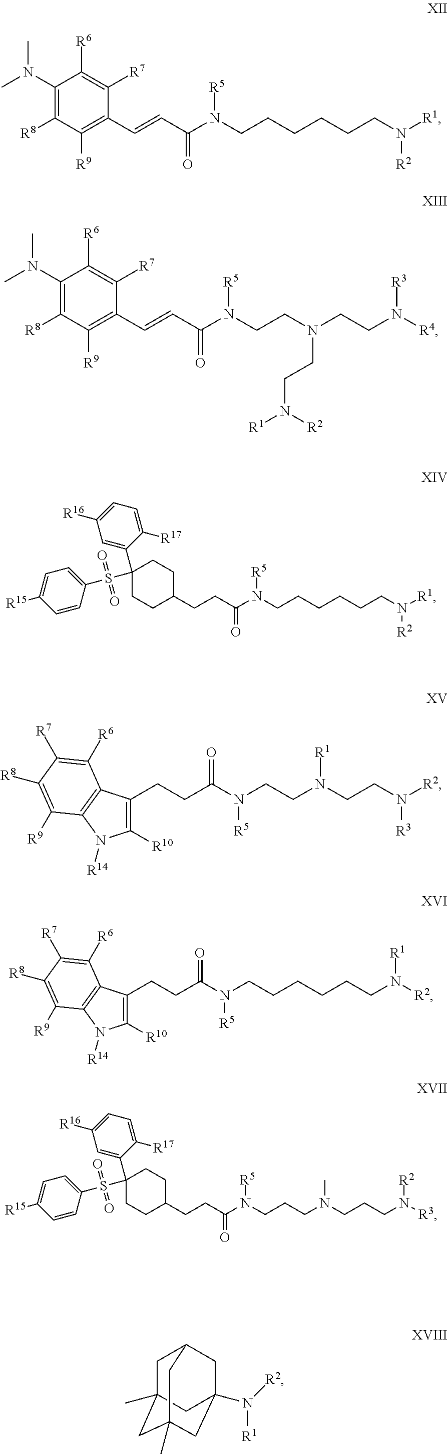 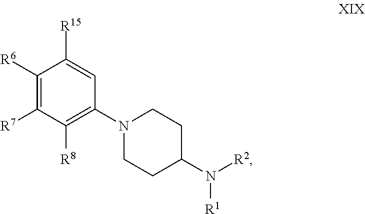
wherein
R¹, R², R³, and R⁴ are each independently a substituted or unsubstituted C₁-C₂₀ alkyl;
R⁵ and R¹⁴ are each independently hydrogen, a substituted or unsubstituted C₁-C₅ alkyl;
R⁶, R⁷, R⁸, R⁹, R¹⁰, R¹¹, R¹², and R¹³ are each independently hydrogen, OH, halogen, or substituted or unsubstituted C₁-C₅ alkyl;
R¹⁵, R¹⁶, and R¹⁷ are each independently hydrogen, OH, halogen, or substituted or unsubstituted C₁-C₅ alkyl; and
R¹⁸ is OH or substituted or unsubstituted C₁-C₁₀ alkyl.

2. (canceled)

3. (canceled)

4. (canceled)

5. (canceled)

6. (canceled)

7. (canceled)

8. (canceled)

9. (canceled)

10. (canceled)

11. (canceled)

12. The compound of claim 1, wherein the compound is represented by any one of Formulas I-A through XIX-A: 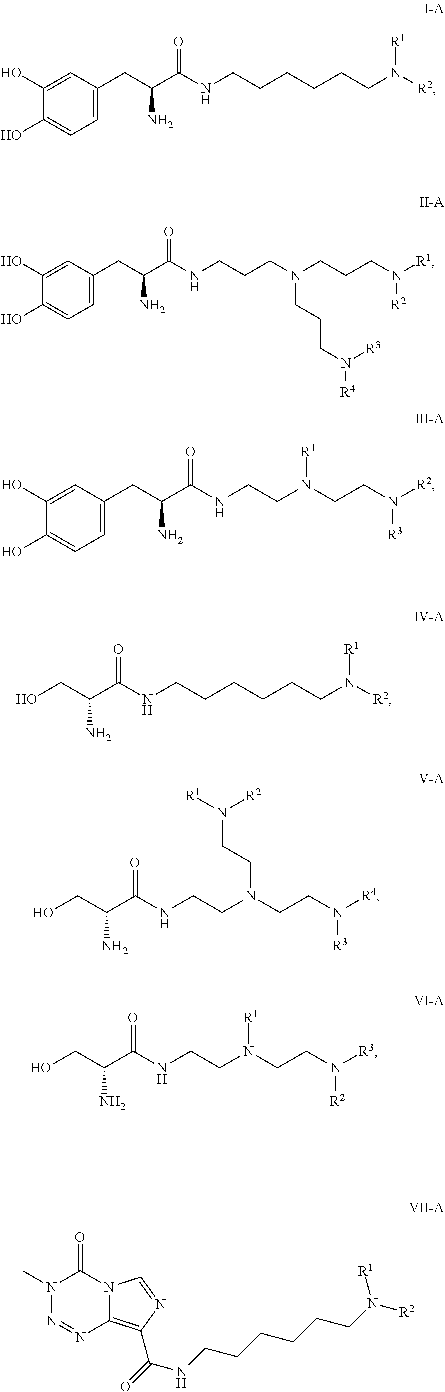 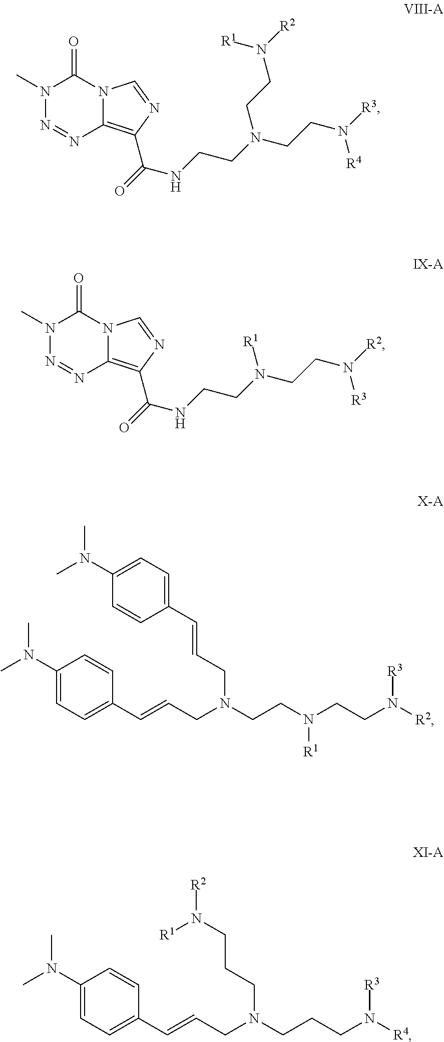 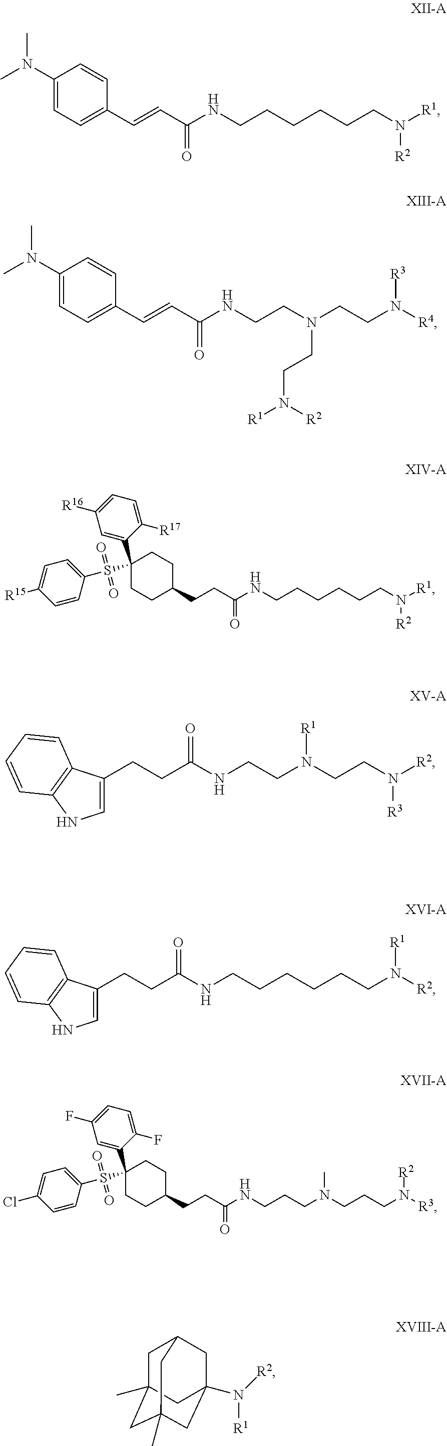 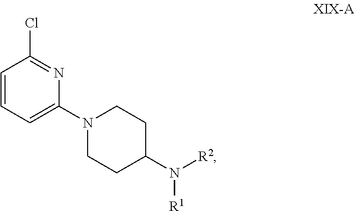
or a pharmaceutically acceptable salt thereof.

13. The compound of claim 1, wherein R¹, R², R³, and R⁴ are each independently a substituted or unsubstituted C₅-C₁₅ alkyl.

14. (canceled)

15. (canceled)

16. The compound of claim 1, wherein R¹, R², R³, and R⁴ are each independently a linear or branched C₅-C₁₅ alkyl substituted with one or more substituents selected from the group consisting of ester, ether, carbonate ester, acetal, ketal, thioketal, thiol, sulfide, disulfide, diols, oxo, and amides.

17. The compound of claim 1, wherein R¹, R², R³, and R⁴ are each independently selected from the group consisting of: 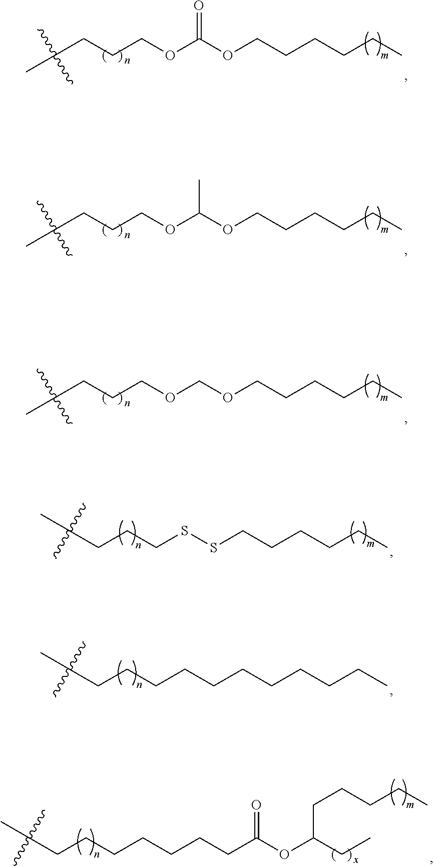
wherein N, M, and X each independently represent integers from 1 to 9.

18. (canceled)

19. The compound of claim 1, wherein at least one of R¹, R², R³, and R⁴ comprise: 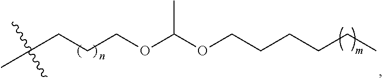
wherein N and M each independently represent integers from 1 to 9.

20. The compound of claim 1, wherein R¹, R², R³, and R⁴ each comprise 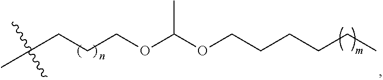
wherein N and M each independently represent integers from 1 to 9.

21. The compound of claim 1, wherein R¹, R², R³, and R⁴ are each independently selected from the group consisting of:

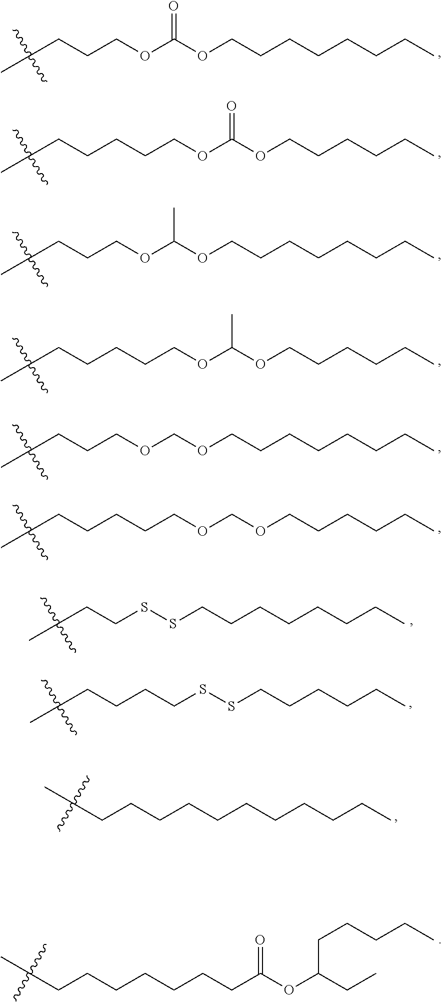

22. (canceled)

23. The compound of claim 1, wherein R¹, R², R³, and R⁴ are all the same.

24. The compound of claim 1, wherein the compound is selected from any one of the structures depicted in BL1-BL83:

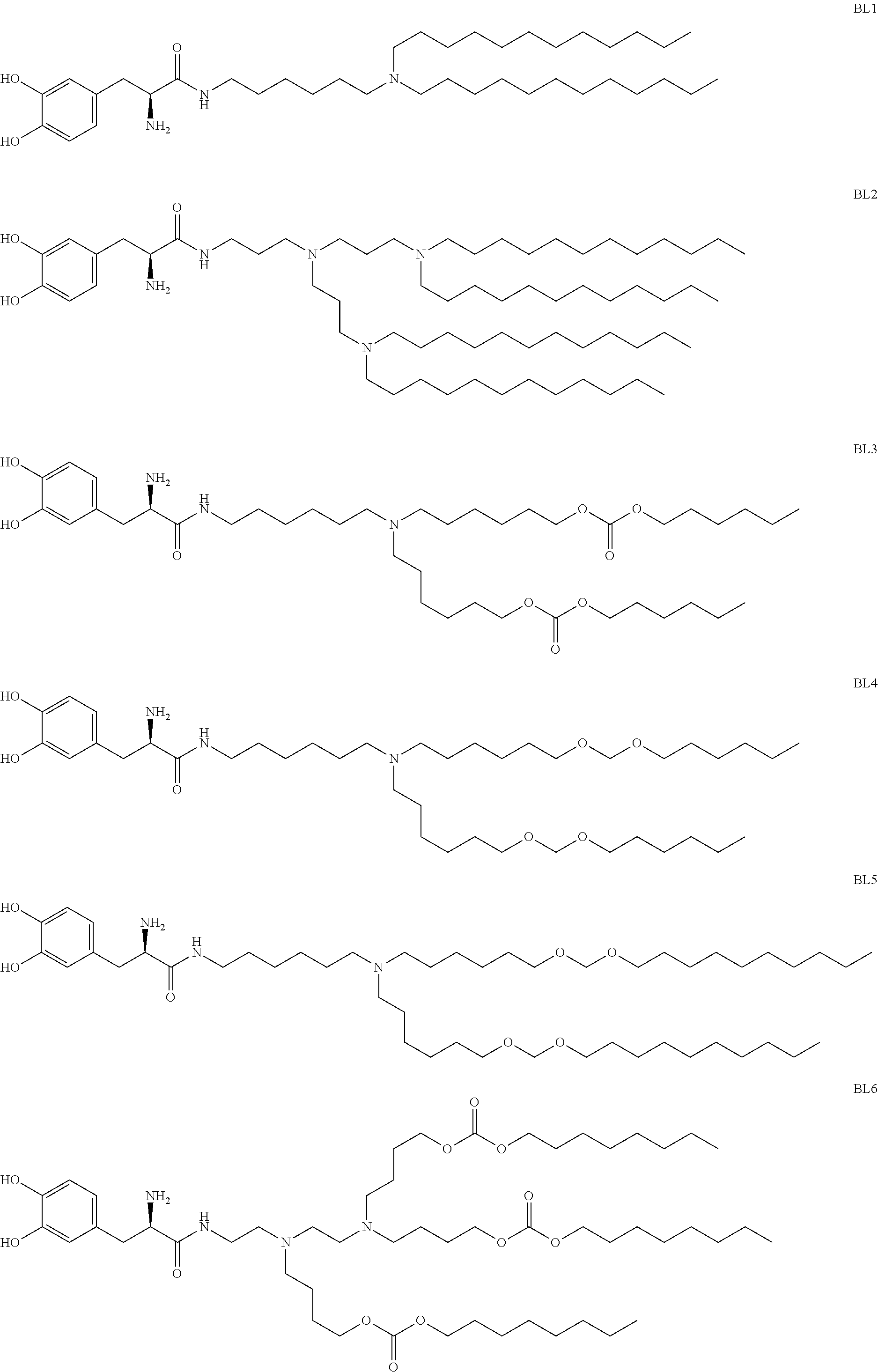 [SMILES: CCCCCCCCCCCCN(CCCCCCCCCCCC)CCCCCCNC(=O)[C@@H](N)Cc1ccc(O)c(O)c1 ; CCCCCCCCCCCCN(CCCCCCCCCCCC)CCCN(CCCNC(=O)[C@@H](N)Cc1ccc(O)c(O)c1)CCCN(CCCCCCCCCCCC)CCCCCCCCCCCC ; CCCCCCOC(=O)OCCCCCCN(CCCCCCNC(=O)[C@H](N)Cc1ccc(O)c(O)c1)CCCCCCOC(=O)OCCCCCC ; CCCCCCOCOCCCCCCN(CCCCCCNC(=O)[C@H](N)Cc1ccc(O)c(O)c1)CCCCCCOCOCCCCCC ; CCCCCCCCCCOCOCCCCCCN(CCCCCCNC(=O)[C@H](N)Cc1ccc(O)c(O)c1)CCCCCCOCOCCCCCCCCCC ; CCCCCCCCOC(=O)OCCCCN(CCCCOC(=O)OCCCCCCCC)CCN(CCCCOC(=O)OCCCCCCCC)CCNC(=O)[C@H](N)Cc1ccc(O)c(O)c1]

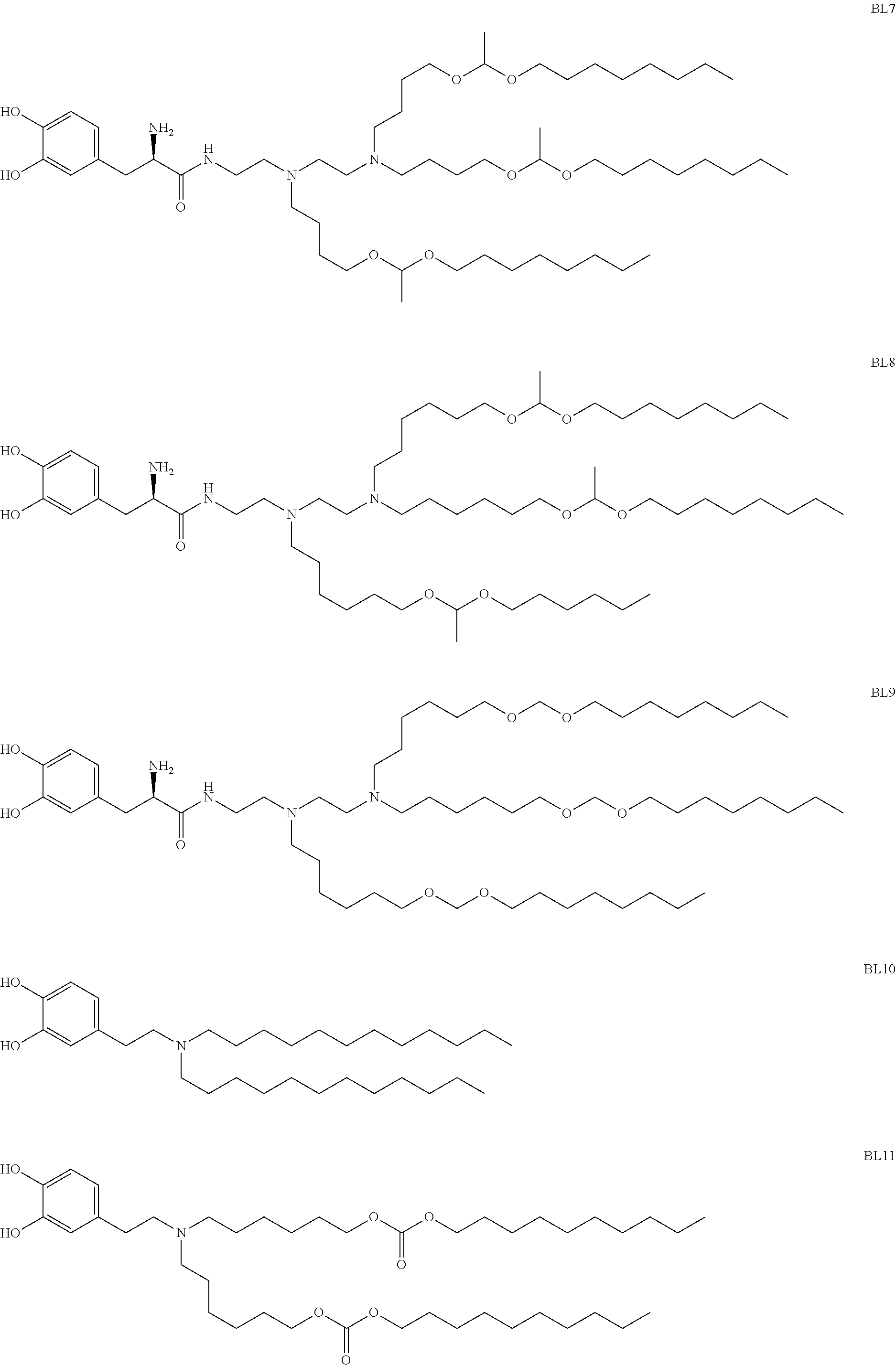 [SMILES: CCCCCCCCOC(C)OCCCCN(CCCCOC(C)OCCCCCCCC)CCN(CCCCOC(C)OCCCCCCCC)CCNC(=O)[C@H](N)Cc1ccc(O)c(O)c1 ; CCCCCCCCOC(C)OCCCCCCN(CCCCCCOC(C)OCCCCCCCC)CCN(CCCCCCOC(C)OCCCCCC)CCNC(=O)[C@H](N)Cc1ccc(O)c(O)c1 ; CCCCCCCCOCOCCCCCCN(CCCCCCOCOCCCCCCCC)CCN(CCCCCCOCOCCCCCCCC)CCNC(=O)[C@H](N)Cc1ccc(O)c(O)c1 ; CCCCCCCCCCCCN(CCCCCCCCCCCC)CCc1ccc(O)c(O)c1 ; CCCCCCCCCCOC(=O)OCCCCCCN(CCCCCCOC(=O)OCCCCCCCCCC)CCc1ccc(O)c(O)c1]

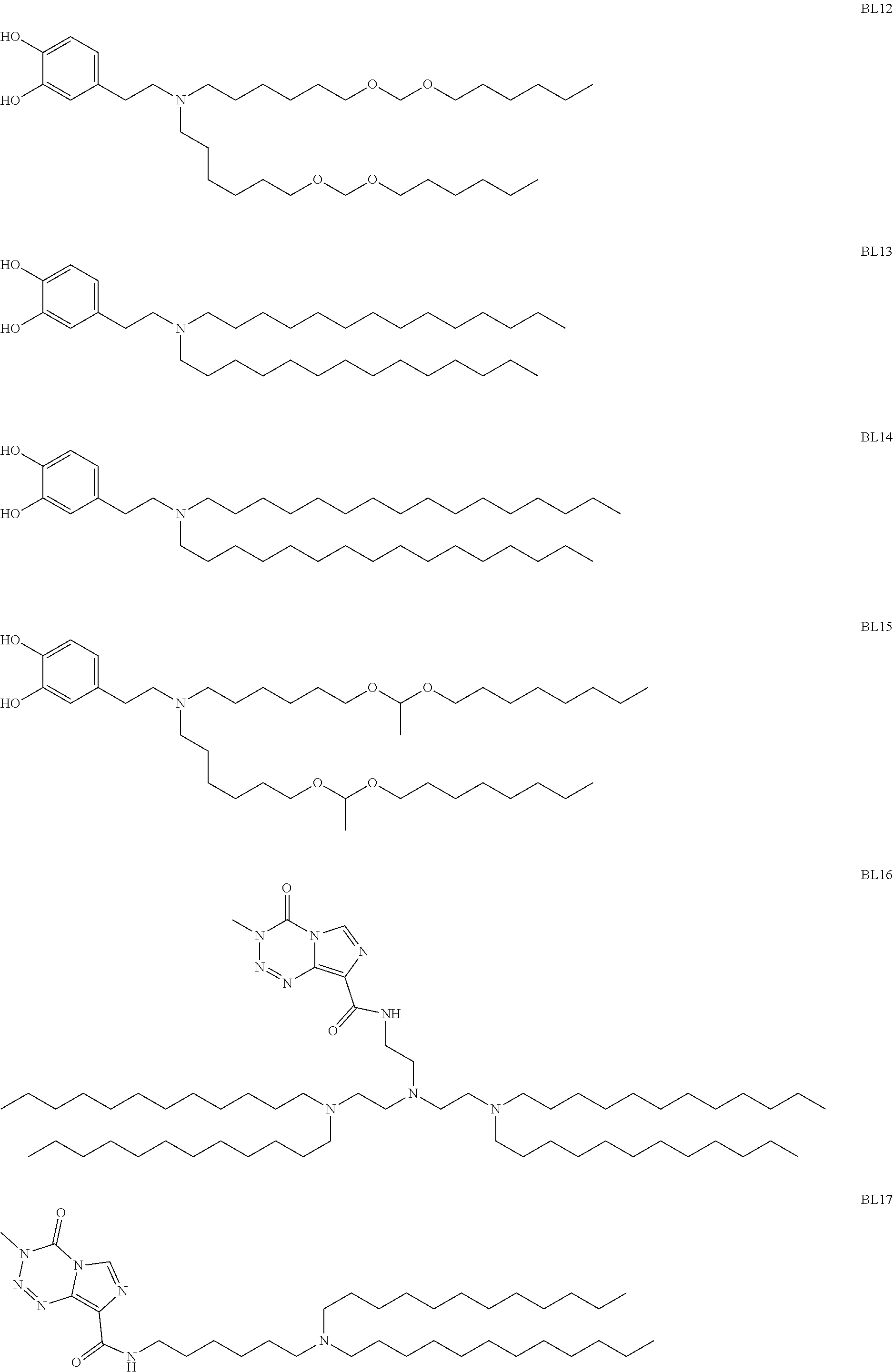

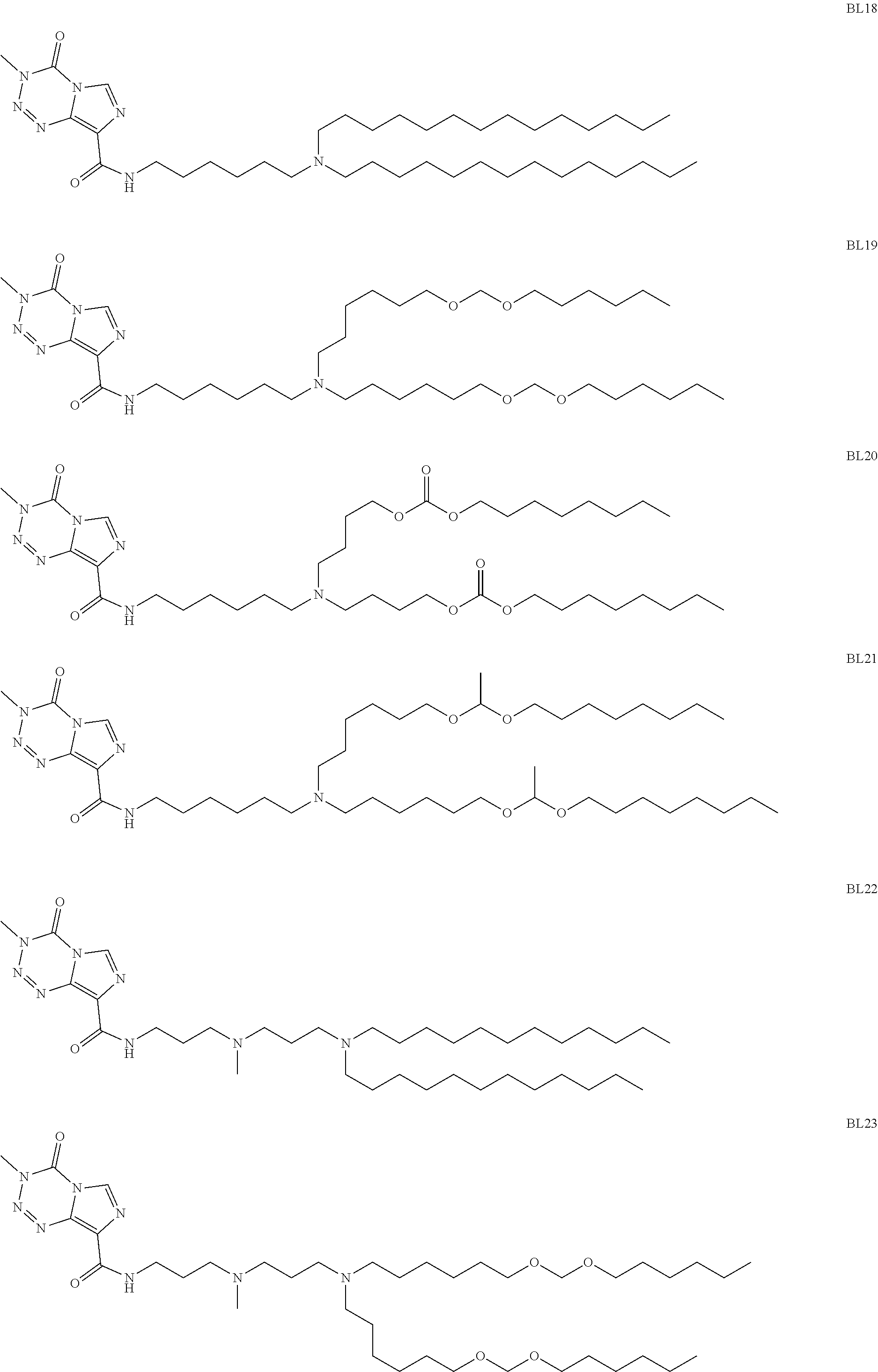

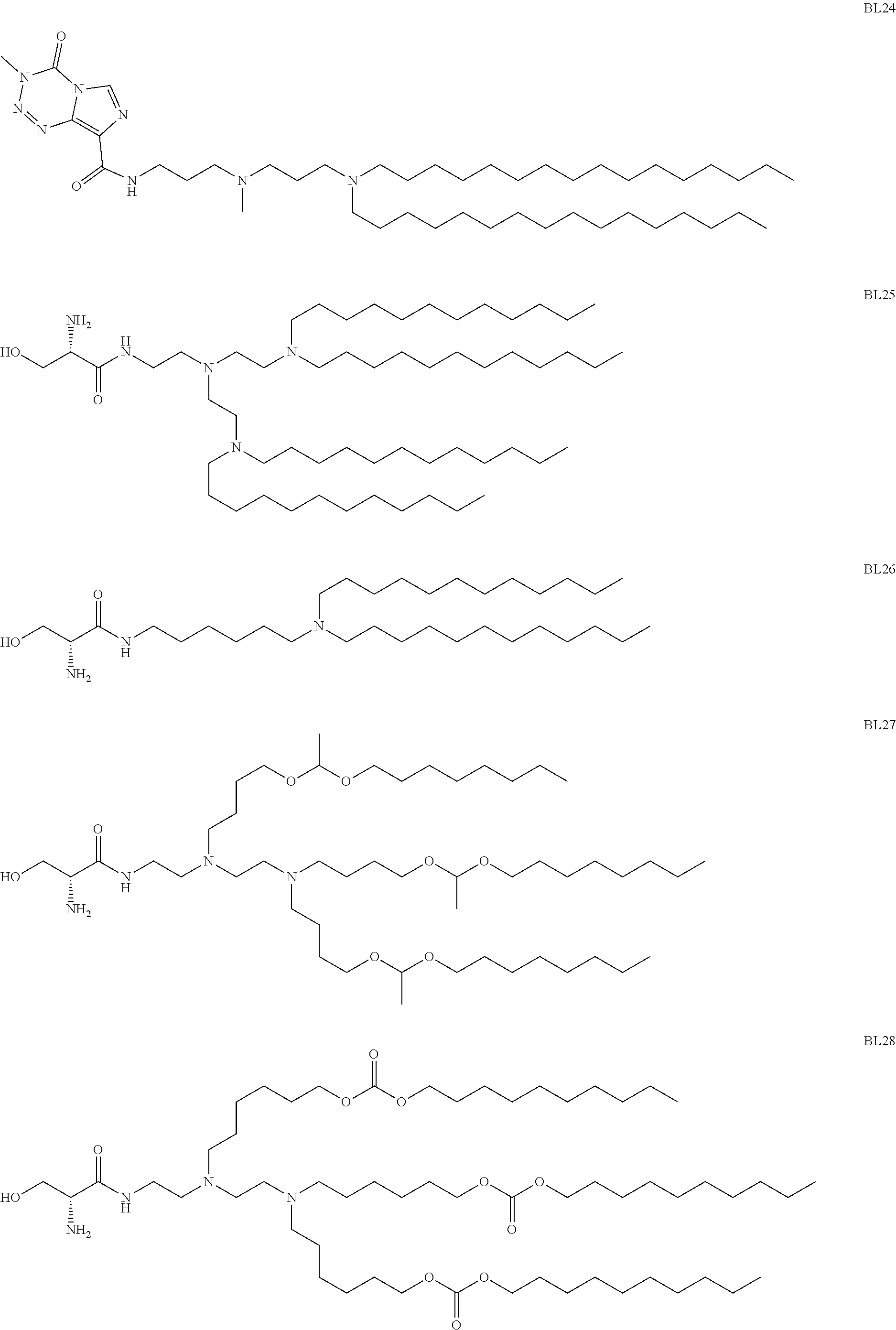 [SMILES: CCCCCCCCCCCCCCCCN(CCCCCCCCCCCCCCCC)CCCN(C)CCCNC(=O)c1ncn2c(=O)n(C)nnc12 ; CCCCCCCCCCCCN(CCCCCCCCCCCC)CCN(CCNC(=O)[C@@H](N)CO)CCN(CCCCCCCCCCCC)CCCCCCCCCCCC ; CCCCCCCCCCCCN(CCCCCCCCCCCC)CCCCCCNC(=O)[C@H](N)CO ; CCCCCCCCOC(C)OCCCCN(CCCCOC(C)OCCCCCCCC)CCN(CCCCOC(C)OCCCCCCCC)CCNC(=O)[C@H](N)CO ; CCCCCCCCCCOC(=O)OCCCCCCN(CCCCCCOC(=O)OCCCCCCCCCC)CCN(CCCCCCOC(=O)OCCCCCCCCCC)CCNC(=O)[C@H](N)CO]

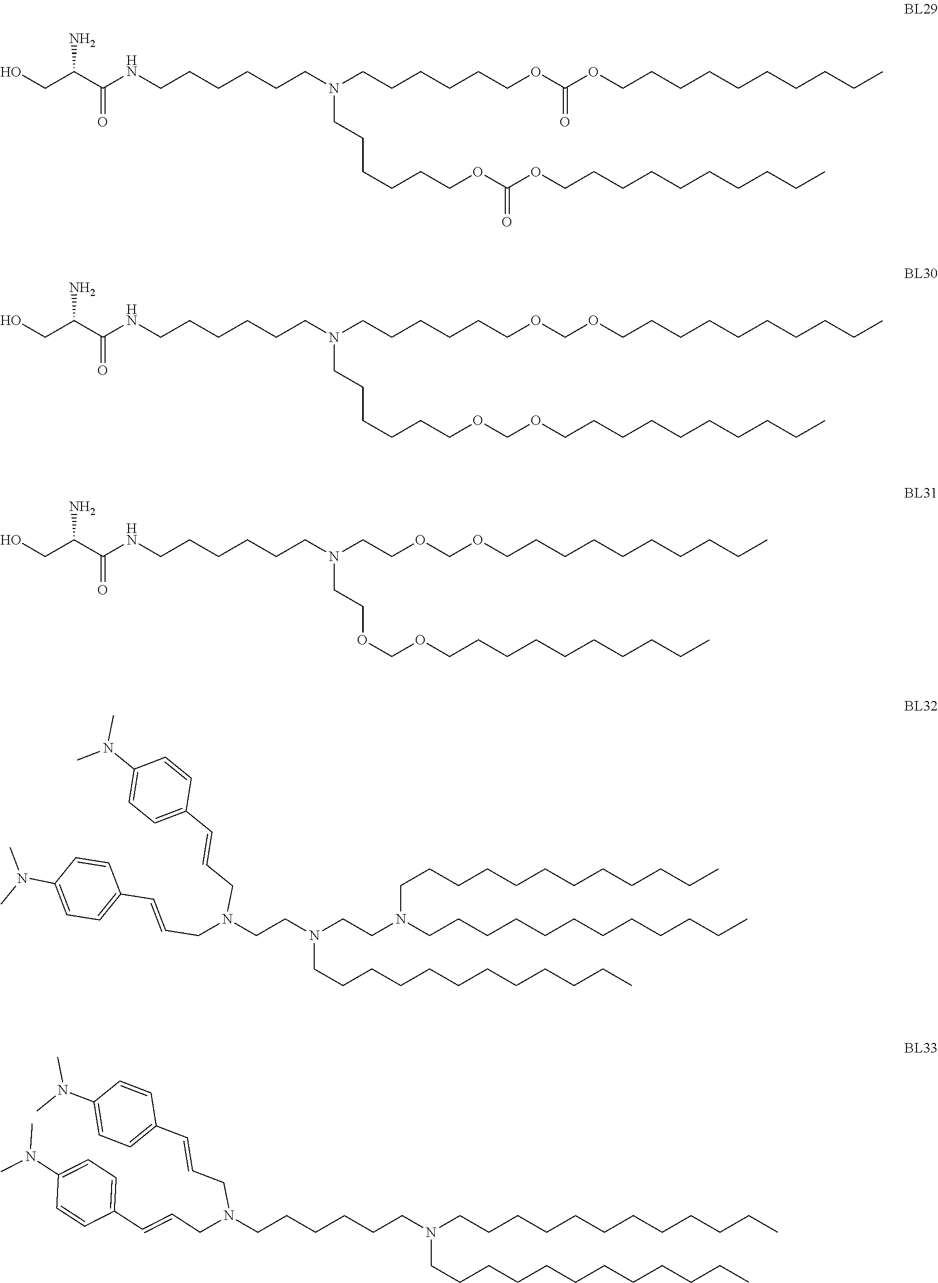

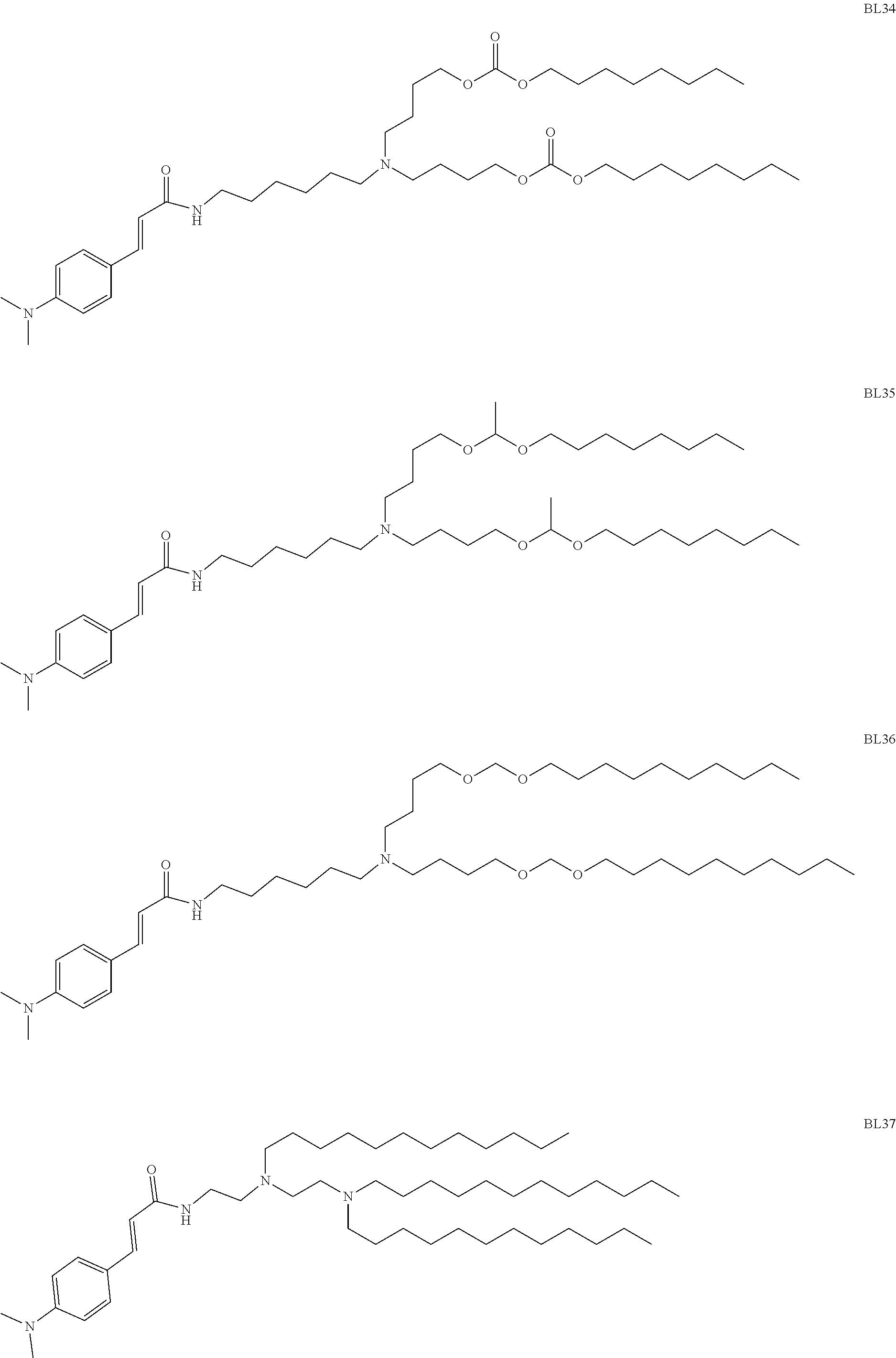

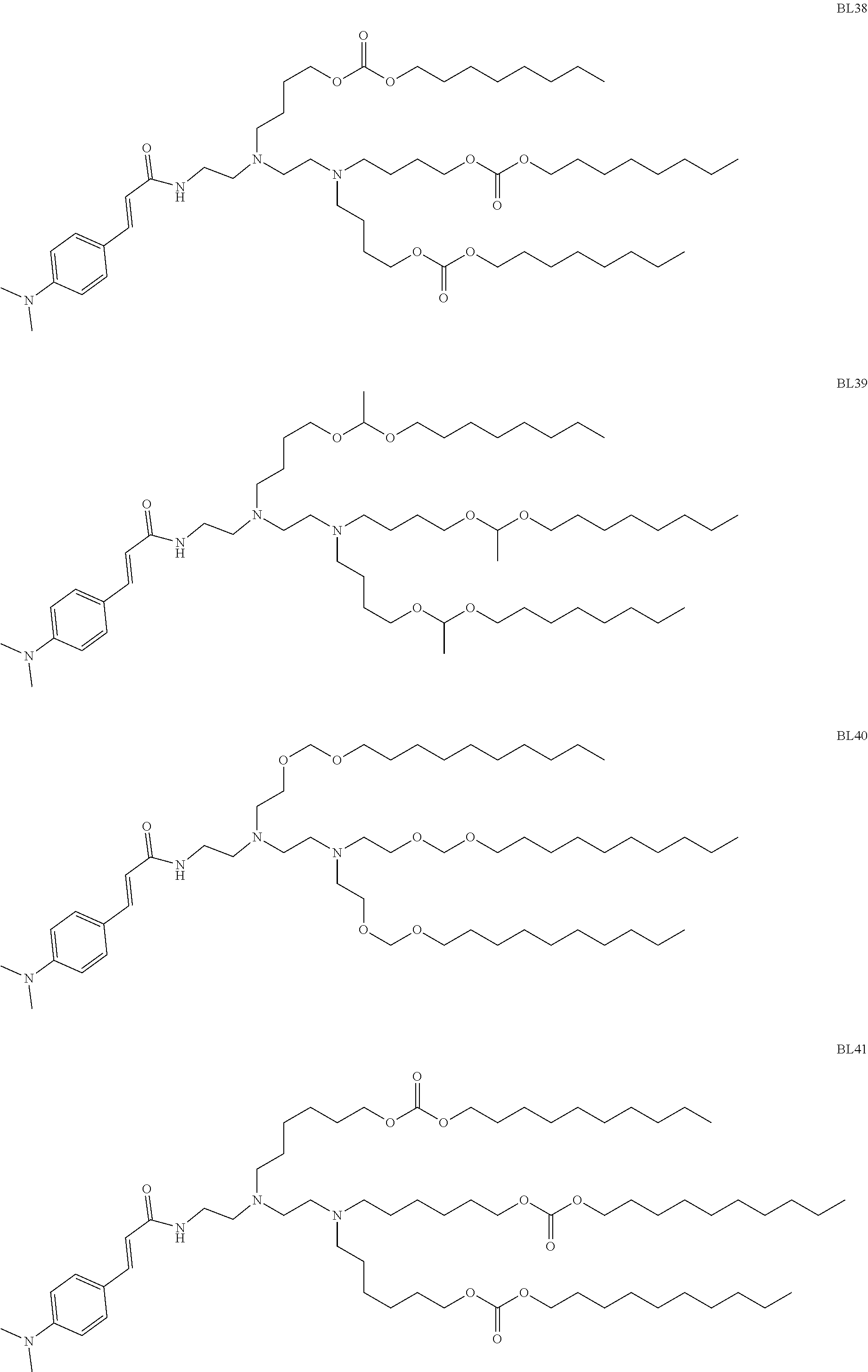

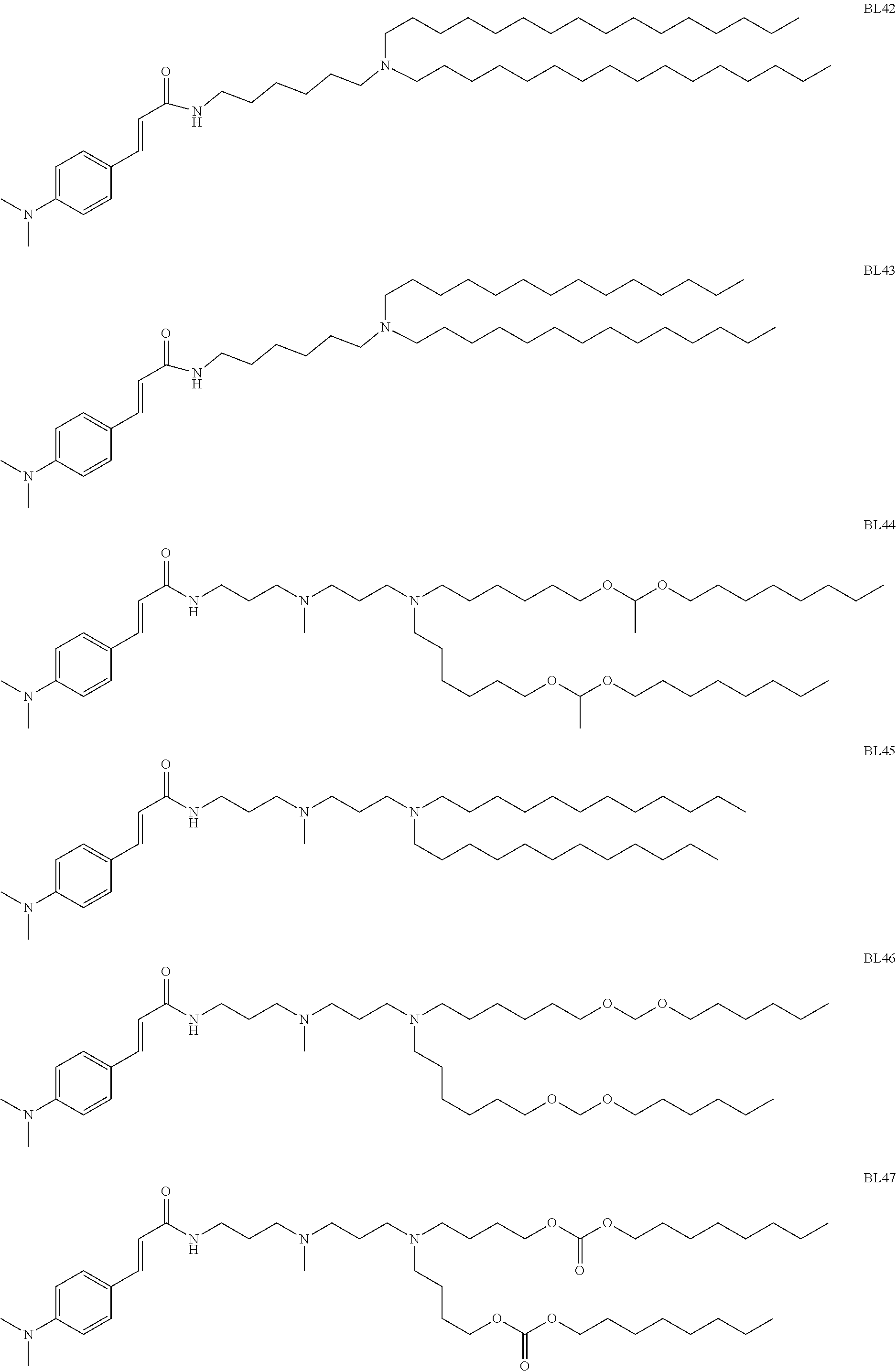

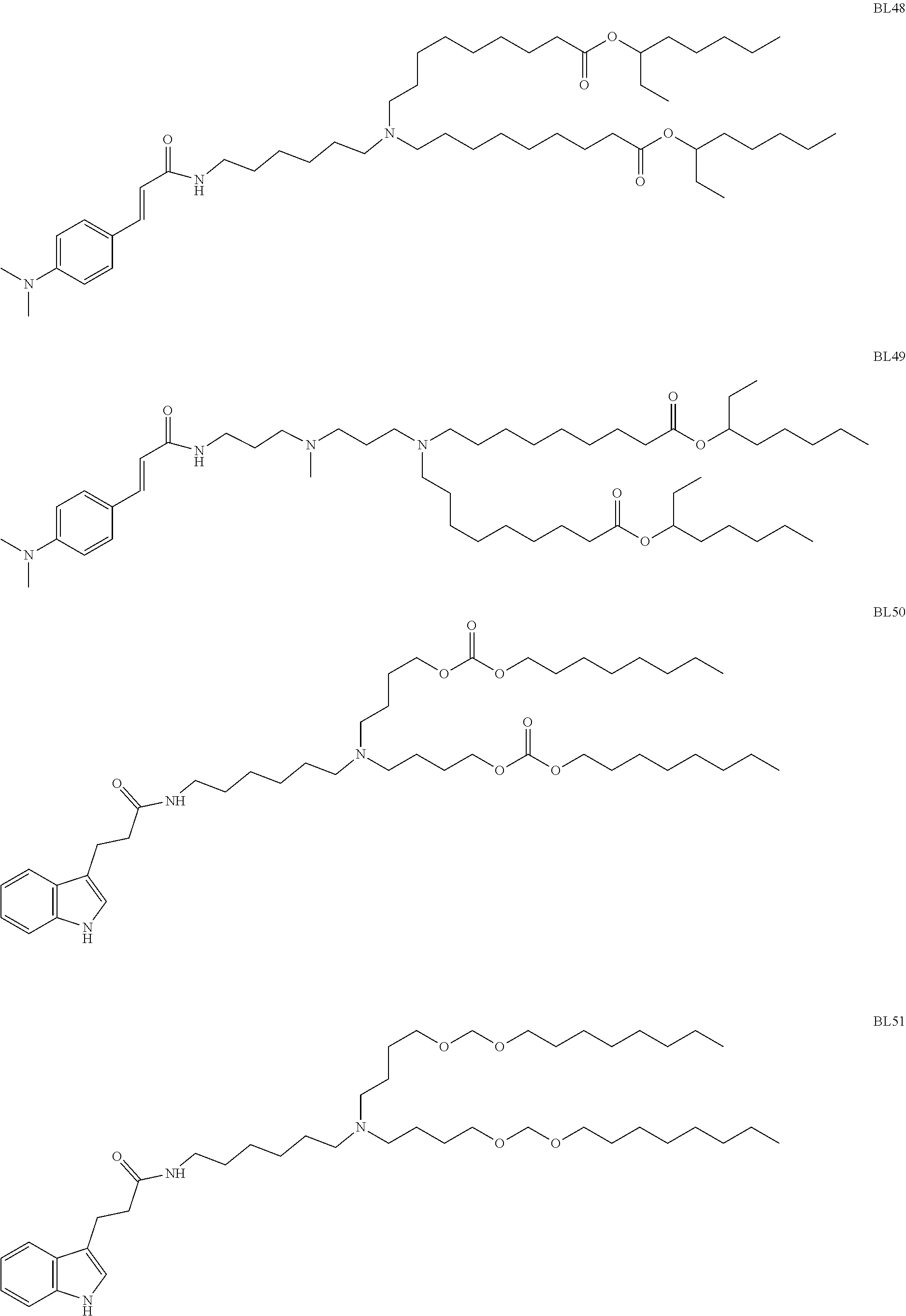

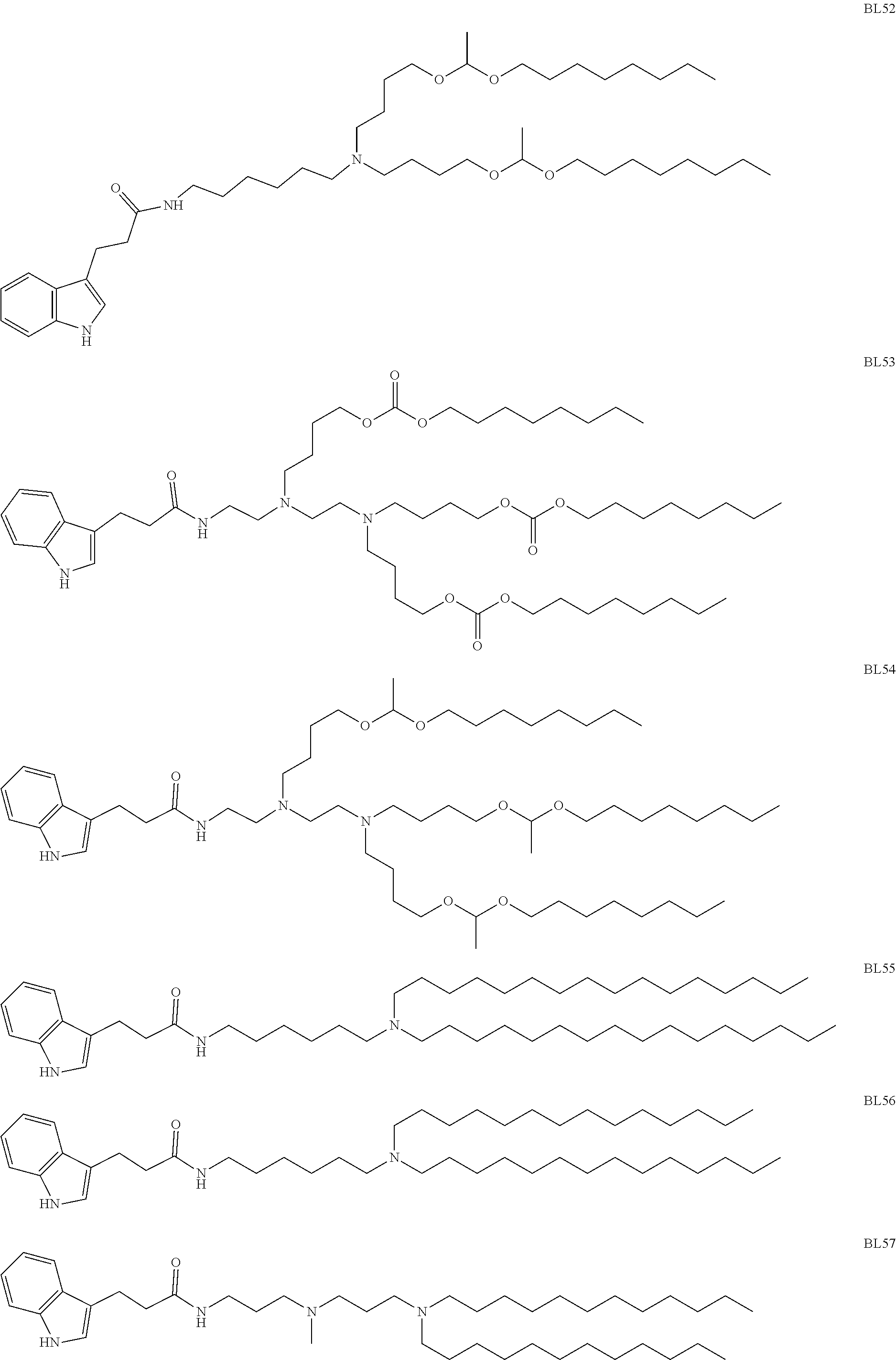

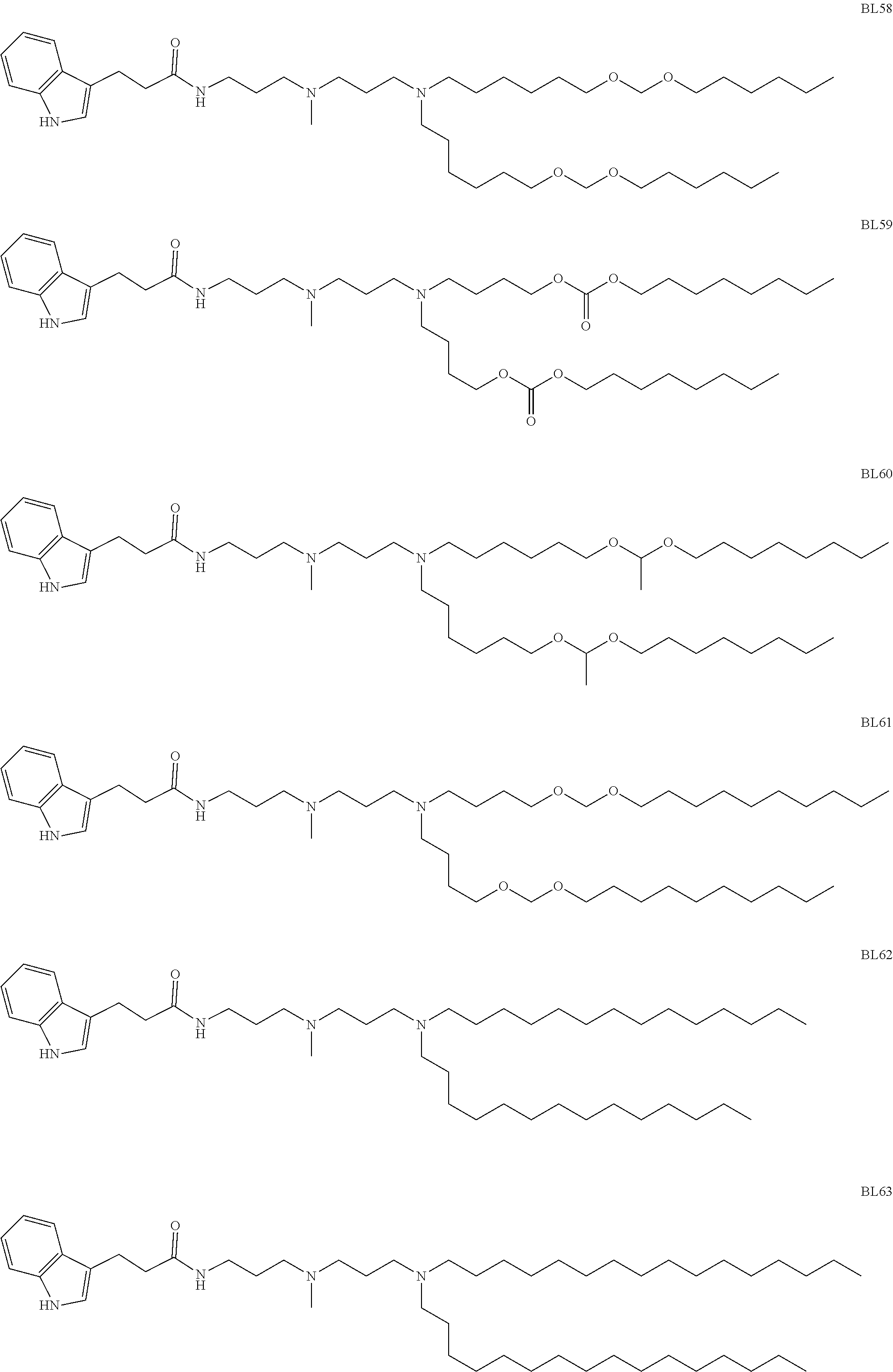 [SMILES: CCCCCCOCOCCCCCCN(CCCCCCOCOCCCCCC)CCCN(C)CCCNC(=O)CCc1c[nH]c2ccccc12 ; CCCCCCCCOC(=O)OCCCCN(CCCCOC(=O)OCCCCCCCC)CCCN(C)CCCNC(=O)CCc1c[nH]c2ccccc12 ; CCCCCCCCOC(C)OCCCCCCN(CCCCCCOC(C)OCCCCCCCC)CCCN(C)CCCNC(=O)CCc1c[nH]c2ccccc12 ; CCCCCCCCCCOCOCCCCN(CCCCOCOCCCCCCCCCC)CCCN(C)CCCNC(=O)CCc1c[nH]c2ccccc12 ; CCCCCCCCCCCCCCN(CCCCCCCCCCCCCC)CCCN(C)CCCNC(=O)CCc1c[nH]c2ccccc12 ; CCCCCCCCCCCCCCCCN(CCCCCCCCCCCCCCCC)CCCN(C)CCCNC(=O)CCc1c[nH]c2ccccc12]

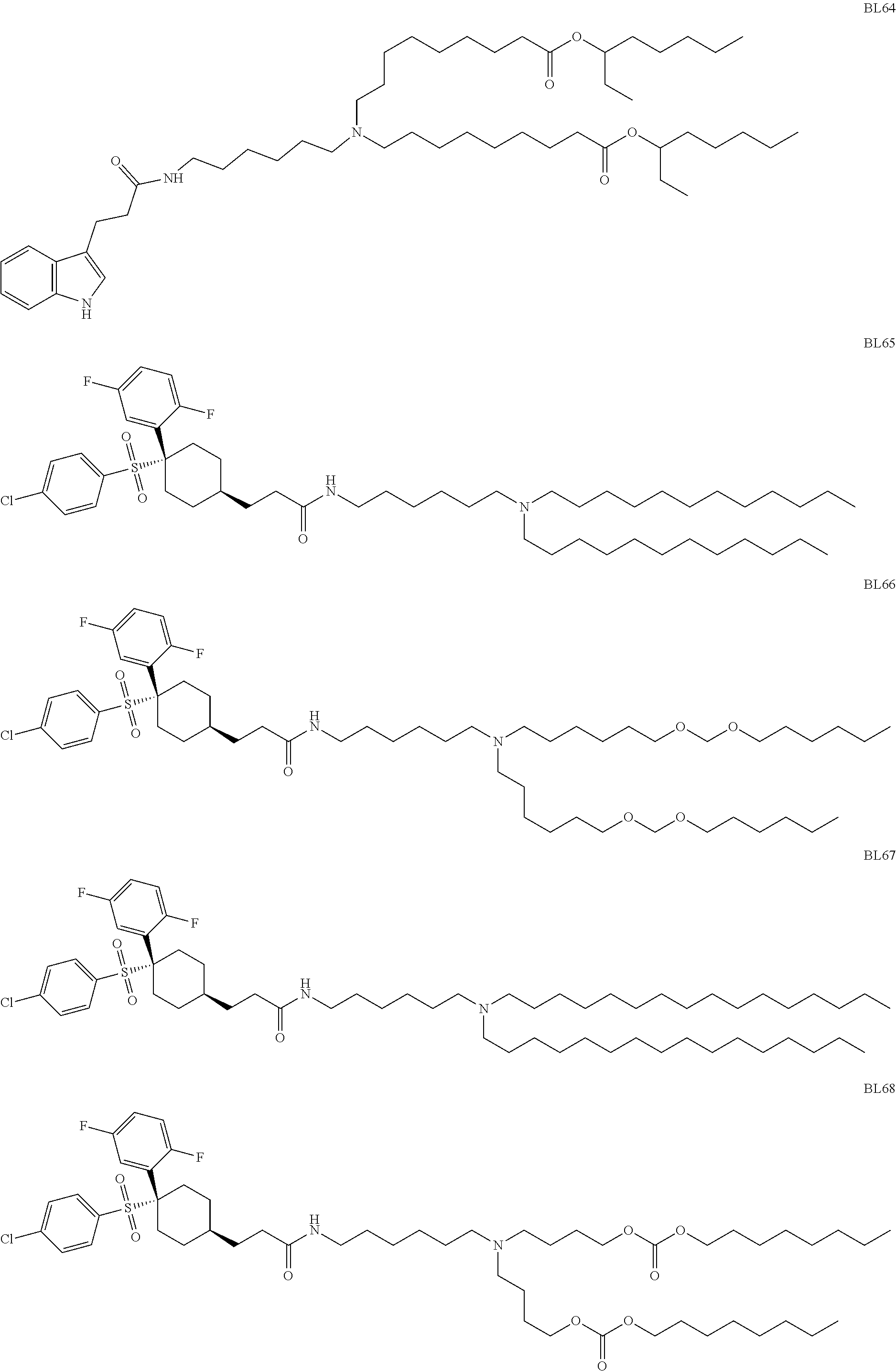 [SMILES: CCCCCC(CC)OC(=O)CCCCCCCCN(CCCCCCCCC(=O)OC(CC)CCCCC)CCCCCCNC(=O)CCc1c[nH]c2ccccc12 ; CCCCCCCCCCCCN(CCCCCCCCCCCC)CCCCCCNC(=O)CC[C@H]1CC[C@](c2cc(F)ccc2F)(S(=O)(=O)c2ccc(Cl)cc2)CC1 ; CCCCCCOCOCCCCCCN(CCCCCCNC(=O)CC[C@H]1CC[C@](c2cc(F)ccc2F)(S(=O)(=O)c2ccc(Cl)cc2)CC1)CCCCCCOCOCCCCCC ; CCCCCCCCCCCCCCCCN(CCCCCCCCCCCCCCCC)CCCCCCNC(=O)CC[C@H]1CC[C@](c2cc(F)ccc2F)(S(=O)(=O)c2ccc(Cl)cc2)CC1 ; CCCCCCCCOC(=O)OCCCCN(CCCCCCNC(=O)CC[C@H]1CC[C@](c2cc(F)ccc2F)(S(=O)(=O)c2ccc(Cl)cc2)CC1)CCCCOC(=O)OCCCCCCCC]

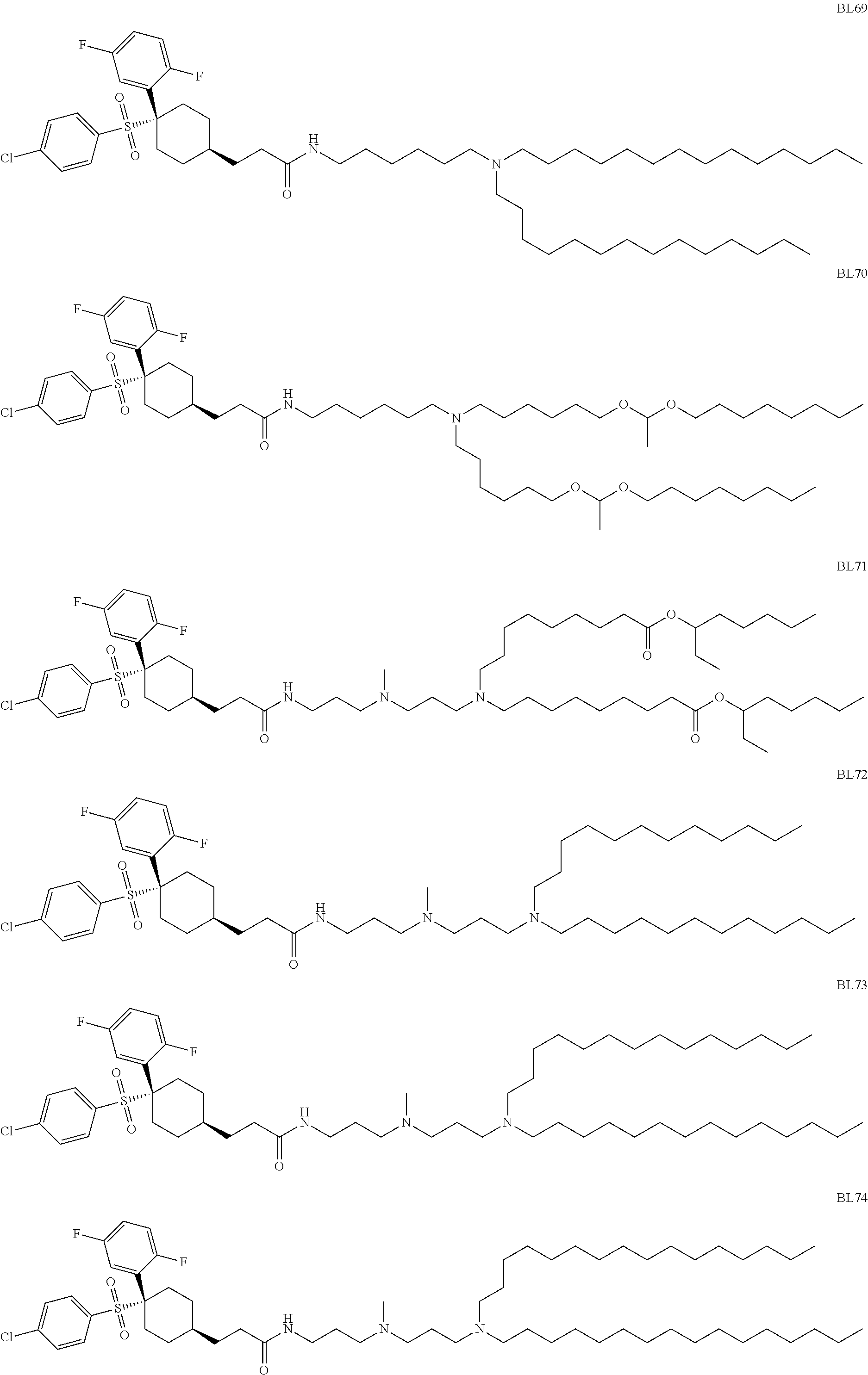

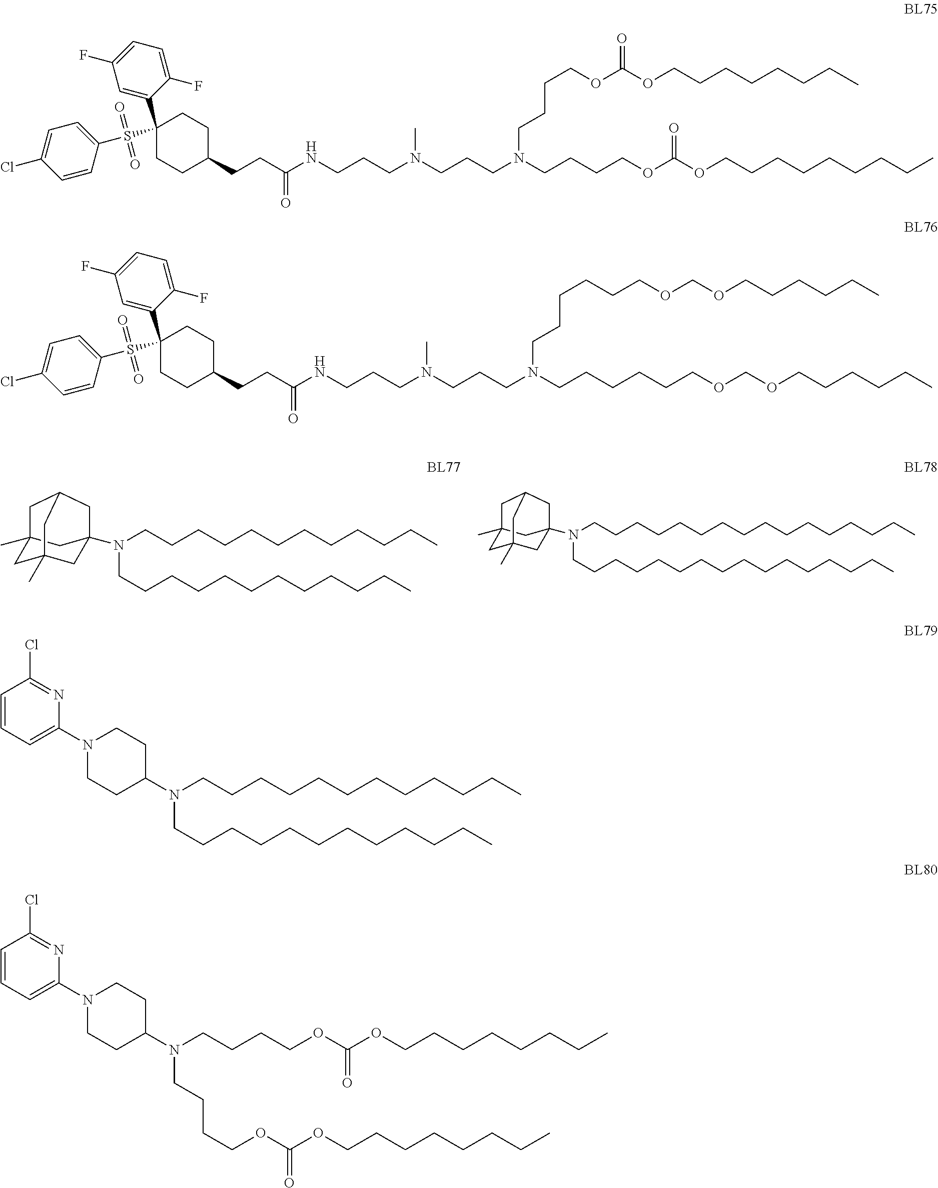

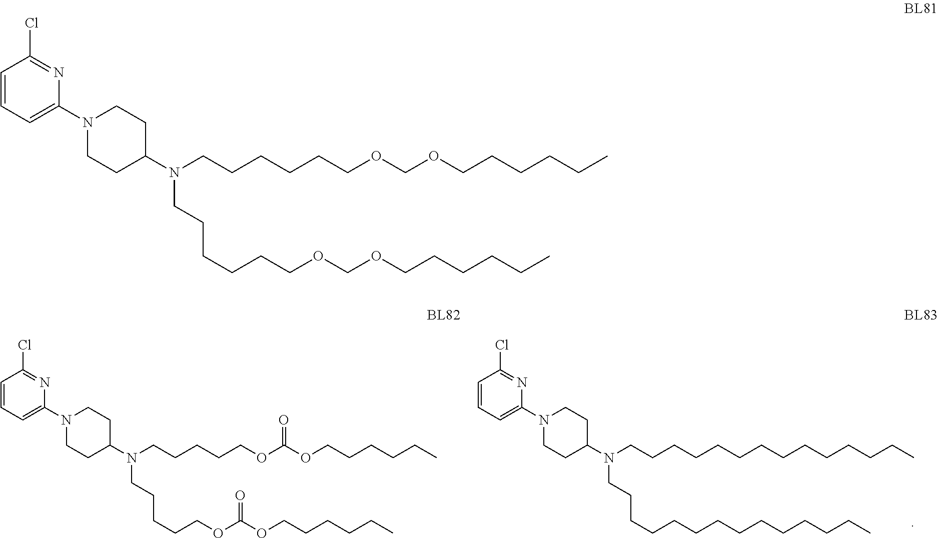 [SMILES: CCCCCCOCOCCCCCCN(CCCCCCOCOCCCCCC)C1CCN(c2cccc(Cl)n2)CC1 ; CCCCCCOC(=O)OCCCCCN(CCCCCOC(=O)OCCCCCC)C1CCN(c2cccc(Cl)n2)CC1 ; CCCCCCCCCCCCCCN(CCCCCCCCCCCCCC)C1CCN(c2cccc(Cl)n2)CC1]

25. (canceled)

26. (canceled)

27. The compound of claim 1, wherein the compound is selected from the group consisting of: 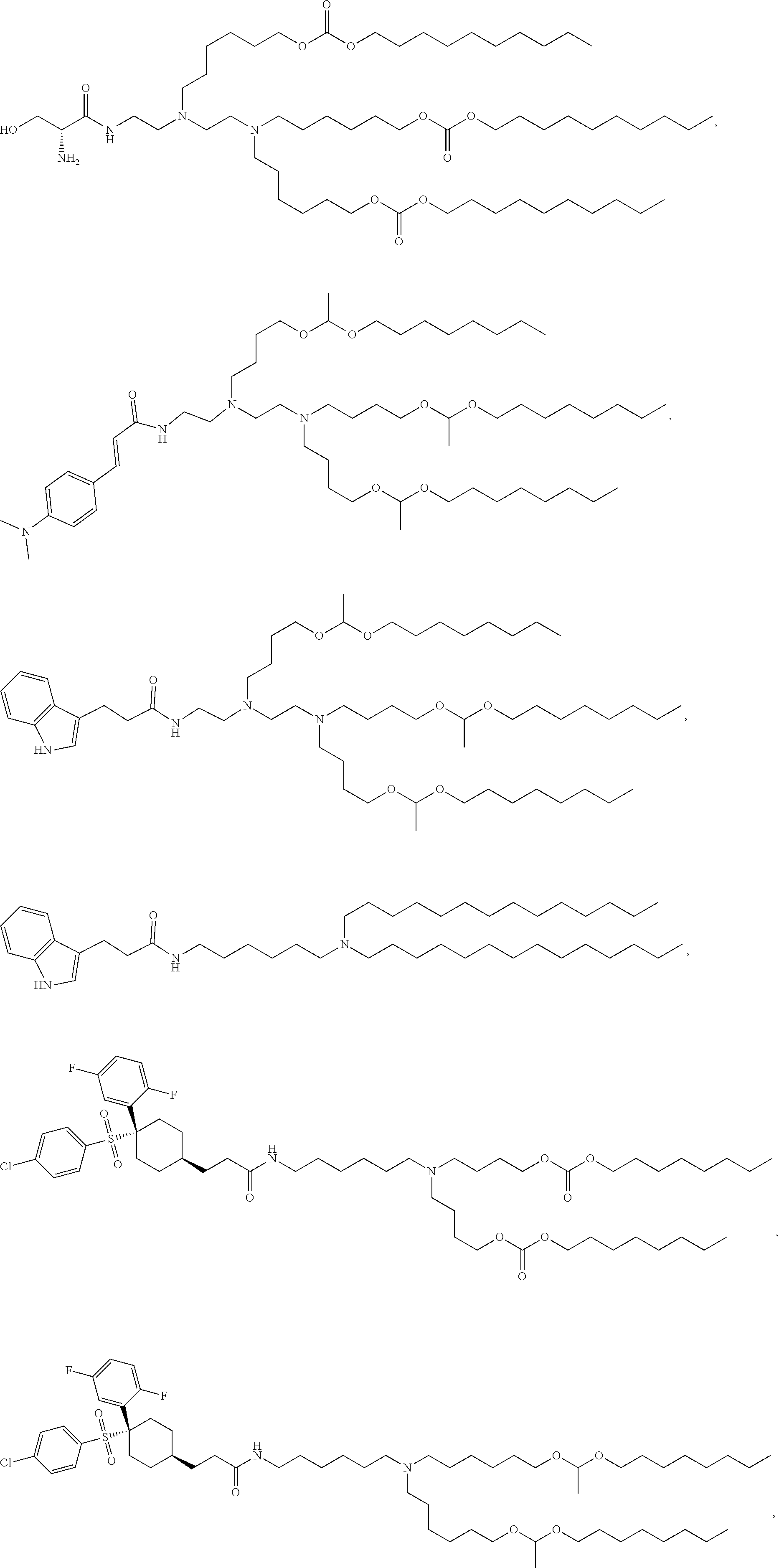
or a pharmaceutically acceptable salt thereof.

28. The compound of claim 1, wherein the compound comprises: 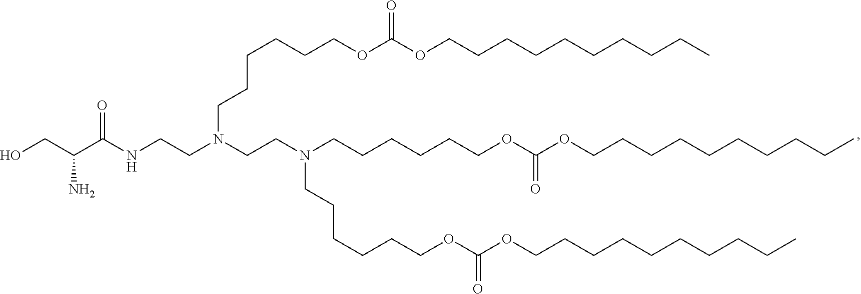 [SMILES: CCCCCCCCCCOC(=O)OCCCCCCN(CCCCCCOC(=O)OCCCCCCCCCC)CCN(CCCCCCOC(=O)OCCCCCCCCCC)CCNC(=O)[C@H](N)CO]
or a pharmaceutically acceptable salt thereof.

29. The compound of claim 1, wherein the compound comprises: 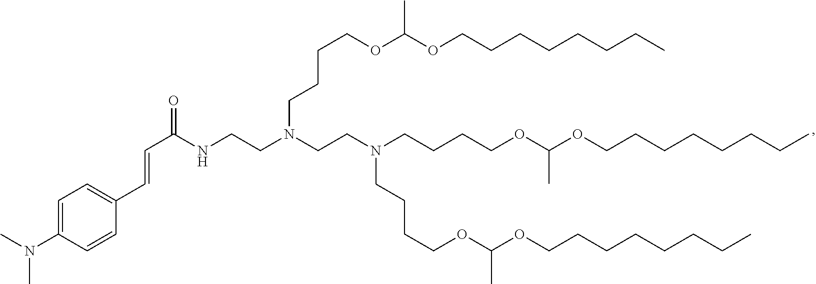 [SMILES: CCCCCCCCOC(C)OCCCCN(CCCCOC(C)OCCCCCCCC)CCN(CCCCOC(C)OCCCCCCCC)CCNC(=O)/C=C/c1ccc(N(C)C)cc1]
or a pharmaceutically acceptable salt thereof.

30. The compound of claim 1, wherein the compound comprises: 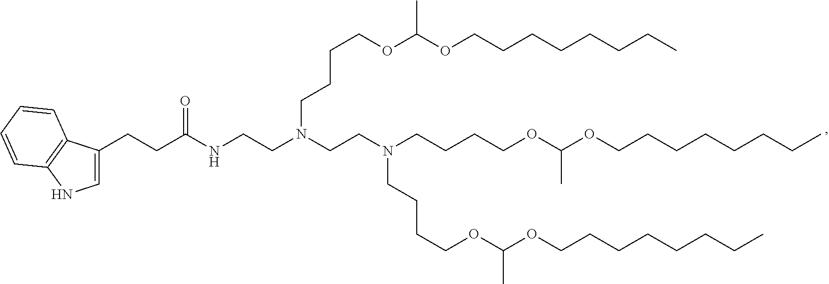 [SMILES: CCCCCCCCOC(C)OCCCCN(CCCCOC(C)OCCCCCCCC)CCN(CCCCOC(C)OCCCCCCCC)CCNC(=O)CCc1c[nH]c2ccccc12]
or a pharmaceutically acceptable salt thereof.

31. The compound of claim 1, wherein the compound comprises: 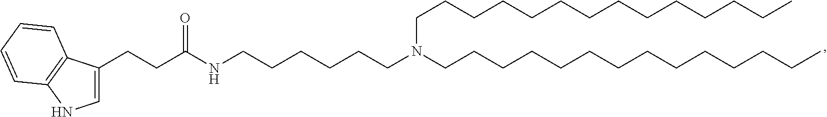 [SMILES: CCCCCCCCCCCCCCN(CCCCCCCCCCCCCC)CCCCCCNC(=O)CCc1c[nH]c2ccccc12]
or a pharmaceutically acceptable salt thereof.

32. The compound of claim 1, wherein the compound comprises: 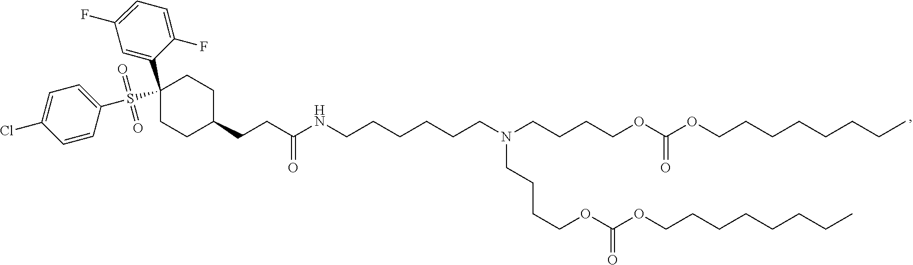 [SMILES: CCCCCCCCOC(=O)OCCCCN(CCCCCCNC(=O)CC[C@H]1CC[C@](c2cc(F)ccc2F)(S(=O)(=O)c2ccc(Cl)cc2)CC1)CCCCOC(=O)OCCCCCCCC]
or a pharmaceutically acceptable salt thereof.

33. The compound of claim 1, wherein the compound comprises: 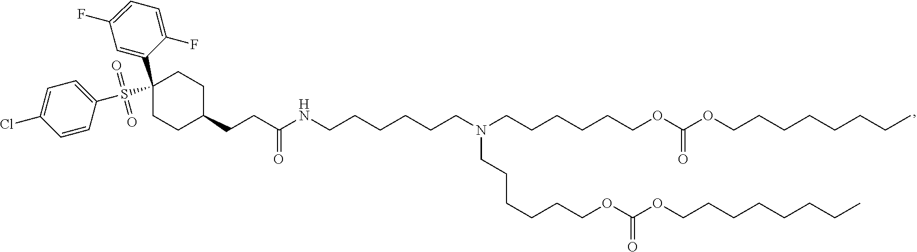 [SMILES: CCCCCCCCOC(=O)OCCCCCCN(CCCCCCNC(=O)CC[C@H]1CC[C@](c2cc(F)ccc2F)(S(=O)(=O)c2ccc(Cl)cc2)CC1)CCCCCCOC(=O)OCCCCCCCC]
or a pharmaceutically acceptable salt thereof.

34. (canceled)

35. A lipid particle comprising the compound of claim 1.

36. (canceled)

37. (canceled)

38. (canceled)

39. (canceled)

40. (canceled)

41. A pharmaceutical composition comprising a therapeutical agent encapsulated within the lipid particle of claim 35.

42. (canceled)

43. (canceled)

44. (canceled)

45. (canceled)

46. (canceled)

47. A method of treating a disease in a subject in need thereof, the method comprising administering to the subject a therapeutically effective amount of the pharmaceutical composition of claim 41.

48. (canceled)

49. (canceled)

50. (canceled)

51. (canceled)

52. (canceled)
